(19) United States
(12) Patent Application Publication
(10) Pub. No.: US 2026/0166114 A1
(43) Pub. Date: Jun. 18, 2026

(54) DYNAMIC HYDROGEL COMPOSITIONS

(71) Applicant: The Board of Trustees of the Leland Stanford Junior University, Stanford, CA (US)

(72) Inventors: Eric A. Appel, Palo Alto, CA (US); Andrea I. D'Aquino, Palo Alto, CA (US); Changxin Dong, Palo Alto, CA (US); Emily Louise Meany, Palo Alto, CA (US); Leslee Nguyen, Palo Alto, CA (US); Ye Eun Song, Stanford, CA (US); Christian Milton Williams, Stanford, CA (US)

(73) Assignee: The Board of Trustees of the Leland Stanford Junior University, Stanford, CA (US)

(21) Appl. No.: 19/126,598

(22) PCT Filed: Nov. 8, 2023
(86) PCT No.: PCT/US2023/079149

§ 371 Date: May 1, 2025

Related U.S. Application Data
(60) Provisional application No. 63/424,059, filed on Nov. 9, 2022.

(51) Int. Cl. A61K 38/00; A61K 9/00; A61K 31/513; A61K 47/34

(52) U.S. Cl. CPC A61K 38/00; A61K 9/0019; A61K 31/513; A61K 47/34

(57) ABSTRACT

Compositions and methods for delivery of therapeutic agents to an individual in need thereof, for prevention/reduction of tissue adhesion, and for 3D printing are disclosed herein. In some embodiments, the composition comprises octadecyl modified hydroxypropyl methylcellulose (HPMC-C18) and a surfactant or cyclic polysaccharide, wherein the composition is a hydrogel that exhibits dynamic properties.

### CROSS-REFERENCE TO RELATED APPLICATION(S)

The present application claims the benefit of priority to U.S. Provisional Application No. 63/424,059, filed Nov. 9, 2022, the contents of which are incorporated by reference in their entirety.

### TECHNICAL FIELD

The present technology relates to dynamic hydrogel compositions and their use in drug delivery, therapeutic treatment, and 3D printing.

### BACKGROUND

There are many disease treatments that can benefit from improved drug delivery, surgical procedures that can be enhanced to reduce tissue adhesion, and 3D-printing processes that can benefit from improved materials. Some of the challenges for each of these issues are discussed below.

For decades, vaccines have played an important role in preventing infectious disease. Subunit vaccines, where only fragments of a pathogen are used to trigger protective immunity, have been shown to be safe and effective in preventing disease. More recently, vaccine therapy utilizing nucleic acids has been developed for treating infectious diseases, cancer, and autoimmune disorders. In cases where challenges to vaccine therapy exist, it is thought to arise from the limited ability to produce robust and persistent immune responses for many target diseases. For instance, a failure of subunit vaccines to elicit a sufficiently strong immune response likely arises, in part, from inappropriate temporal control over antigen presentation and adjuvant mediated activation. Further, vaccines and other immunotherapies are typically administered in a saline solution as a series of multiple shots to achieve appropriate responses. In instances where vaccine delivery relies on polymer microparticles, residual organic solvents from polymer microparticle synthesis can denature biologic cargo. Conventional approaches for delivering nucleic acid vaccines may result in reduced exposure to intended immune cells when the nucleic acid vaccine is taken up by non-immune cells, thereby reducing efficacy. Therefore, there is a significant need for new materials that enable sustained exposure of vaccine and/or immunotherapy components, particularly of multiple compounds of various sizes, to the immune system.

Diabetes or pre-diabetes affects approximately 500 million people worldwide, including an estimated 130 million individuals in the U.S. Treatments include the use of incretin mimetics, which mimic naturally produced incretin hormones to stimulate insulin release in response to a meal. Conventional treatments with incretin mimetics involve daily or weekly injections, which is a significant patient burden and results in poor compliance and can result in dangerous hypoglycemic events. Therefore, there is a significant need for a drug delivery system that can release diabetic therapies in a sustained and controlled manner after administration of a single dose.

Over 20 million Americans undergo invasive surgery each year, and adhesions develop after 95% of all operations, regardless of the procedure or location on the body. Current solutions available for commercial use are typically polymer films based on polysaccharides and/or synthetic polymers (both resorbable and non-resorbable varieties), which serve as a physical barrier between scarring tissue and surrounding organs. Other technologies that have been tested are based on sprayable pre-polymer solutions that polymerize into polymeric hydrogel films in situ, or which are simple polymer solutions. For polymer solutions comprising, for example, chitosan, hyaluronic acid (HA) and/or carboxymethylcellulose (CMC), the residence time at the injured tissues is too short. For resorbable solid membranes including HA-CMC (Seprafilm, Genzyme, Cambridge, MA) and polylactide, it is difficult to completely cover the affected tissues during application, which is particularly problematic in areas of the body with many surfaces that may form adhesions, such as in the abdomen. Despite the overwhelming need, current adhesion barrier technologies have not been widely adopted due to their inefficacy to fully limit adhesions, rapid degradation time, and difficulty handling during surgery.

Additive manufacturing, more commonly known as 3D printing, has revolutionized the way products are manufactured, but many improvements are still needed. One drawback is that 3D printing is typically a slow process. For example, in additive manufacturing processes that involve forming an object from an extruded material filament, the filament may break under high printing speeds. Further challenges in 3D printing of biocompatible materials include maintaining continuous filaments (especially if the filament has a small diameter), providing good shape and resolution, and maintaining the shape of the printed object over time. Thus, there is a need for a biocompatible material with desirable extrudability and printability properties that can be used in 3D printing.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIGS. 1A-IE illustrates the results of material characterization of Sangelose and Tween 80 hydrogels. FIG. 1A shows that a hydrophobically-modified hydroxypropyl methylcellulose polymer (Sangelose) forms a modular and dynamic hydrogel when mixed with the surfactant Tween 80. FIG. 1B illustrates oscillatory frequency sweeps of hydrogels with increasing Tween 80 weight percent (1.5 wt % Sangelose (S1.5), 1.5 wt % Sangelose and 0.1 wt % Tween 80 (S1.5Tw₈₀0.1), 1.5 wt % Sangelose and 0.5 wt % Tween 80 (S1.5Tw₈₀0.5), and 1.5 wt % Sangelose and 1 wt % Tween 80 (S1.5Tw₈₀1)). FIG. 1C shows oscillatory amplitude sweeps of various hydrogels with increasing Tween 80 concentrations. FIG. 1D plots static yield stress of hydrogels determined by stress-controlled flow sweeps (n=4). FIG. 1E plots step shear measurements of high (10 s⁻¹) and low (0.1 s⁻¹) shear rates in 30-s steps.

FIGS. 2A and 2B illustrate extensional rheology of Sangelose and Tween 80 hydrogels. FIG. 2A shows extensional strain-to-break measurements of Sangelose hydrogels with increasing Tween 80 weight percent (S1.5, S1.5Tw₈₀0.1, S1.5Tw₈₀0.5, and S1.5Tw₈₀1) at four different strain rates. Strain-to-break shows a positive correlation with Tween 80 concentration. One-way ANOVA analysis with post hoc Tukey test was used to measure p-values (n=4). FIG. 2B provides images of Sangelose and Tween 80 hydrogels before the break at 0.3 s⁻¹ strain rate. Strain-to-break measurements were performed as denoted in the images.

FIGS. 3A-3F illustrate the results of material characterization of Sangelose and α-cyclodextrin (αCD). FIG. 3A shows that Sangelose forms a modular and dynamic hydrogel when mixed with the cyclic polysaccharide αCD. FIG. 3B illustrates oscillatory sweeps of a high weight percent Sangelose αCD hydrogel (3 wt % Sangelose and 0.02 wt % αCD (S3αCD0.02)) and a low weight percent Sangelose αCD hydrogel (1.8 wt % Sangelose and 0.023 wt % αCD (S1.8αCD0.023)). FIG. 3C shows flow sweeps of S3αCD0.02 and S1.8αCD0.023 hydrogels. FIG. 3D plots static yield stress of hydrogels determined by stress-controlled flow sweeps. FIG. 3E shows oscillatory sweeps at 0.1 rad·s⁻¹. FIG. 3F plots step shear measurements of high (10 s⁻¹) and low (0.1 s⁻¹) shear rates in 30 s steps.

FIGS. 4A-4F illustrate the results of material characterization of Sangelose and Tween 20 hydrogels. FIG. 4A shows that Sangelose forms a modular and dynamic hydrogel when mixed with the surfactant Tween 20. FIG. 4B illustrates oscillatory frequency sweeps of a Sangelose only hydrogel (S3) and Sangelose hydrogels with Tween 20 (STw₂₀0.75). FIG. 4C shows flow sweeps of S3 and STw₂₀0.075 hydrogels. Both hydrogels displayed shear-thinning behavior with decreasing viscosity at high shear rates. FIG. 4D plots static yield stress of hydrogels determined by stress-controlled flow sweeps (n=3). FIG. 4E shows oscillatory sweeps of S and STw₂₀0.075 hydrogels at 0.1 rad·s⁻¹. FIG. 4F plots step shear measurements of high (10 s⁻¹) and low (0.1 s⁻¹) shear rates in 30-s steps. Both hydrogels displayed a viscosity decrease of two orders of magnitude at a high shear rate, and restored mechanical properties when the shear stress was removed.

FIGS. 5A-5C illustrate the humoral immune response following immunization with varicella-zoster virus (VZV) gE protein. FIG. 5A provides the vaccination and experiment timeline. FIG. 5B plots the weekly total anti-gE specific IgG ELISA titers from Day 7 to Day 70 of hydrogels formulated with 3 wt % Sangelose only (S3), 3 wt % Sangelose and 0.75 wt % Tween 20 (S3Tw₂₀0.75), and 3 wt % Sangelose and 0.02 wt % αCD (S3αCD0.02), compared with that of the bolus control. FIG. 5C shows the area under the curve (AUC) of FIG. 5B post-immunization.

FIG. 6A is a schematic illustration of micelle formation of the hydrophobic side chains and hydrophilic backbone of hydrophobically modified hydroxypropyl methylcellulose (HPMC-C18, Sangelose) (top) and mixed micelle formation after addition of a second agent, which has the ability to disperse within the micelle and influence the crosslink dynamic of the Sangelose hydrophobic side chains (bottom).

FIGS. 6B-6D are plots showing rheological measurements of hydrogels with varying concentrations of Sangelose (1.5 wt % (S1.5), 2 wt % (S2), 2.5 wt % (S2.5), and 3 wt % (S3)): frequency-dependent oscillatory shear sweep (FIG. 6B), storage modulus (G′) (FIG. 6C), and tan (delta) (FIG. 6D) measured at 10⁻¹ rad·s⁻¹ from 6B.

FIGS. 6E-6H are plots showing the frequency-dependent oscillatory shear rheology of hydrogel compositions with Sangelose combined with various second agents: Tween 20 (S3Tw₂₀0.75) (FIG. 6E), Tween 80 (S3Tw₈₀0.75) (FIG. 6F), α-cyclodextrin (S3aCD0.05) (FIG. 6G), and Span 20 (S3Span₂₀0.75) (FIG. 6H).

FIGS. 6I and 6J are plots of Storage modulus (G′) (FIG. 6I) and tan (delta) (FIG. 6J) of Sangelose based hydrogels with Tween 20 (S3Tw₂₀0.75), Tween 80 (S3Tw₈₀0.75), αCD (S3aCD0.05), Span 20 (S3Span₂₀0.75), or no second agent (S3).

FIGS. 7A-7H are plots showing rheological measurements of Sangelose-based hydrogel compositions with varying second agents: frequency-dependent oscillatory shear sweep of compositions comprising Tween 20 (S3Tw₂₀0.75) (FIG. 7A), Tween 40 (S3Tw₄₀0.75) (FIG. 7B), Tween 60 (S3Tw₆₀0.75) (FIG. 7C), Tween 80 (S3Tw₈₀0.75) (FIG. 7D), Tween 65 (S3Tw₆₅0.75) (FIG. 7E), and Tween 85 (S3Tw₈₀0.75) (FIG. 7F). Comparison of the storage modulus (G′) (FIG. 7G) and tan (delta) (FIG. 7H) values for each formulation measured at 10⁻¹ rad·s⁻¹ from the data shown FIGS. 7A-7F.

FIGS. 8A-8F are plots showing rheological measurements of Sangelose-Tween 80 hydrogel compositions with 3 wt % Sangelose and increasing concentrations of Tween 80 (0.1 wt % (S3Tw₈₀0.1), 0.5 wt % (S3Tw₈₀0.5), 1 wt % (S3Tw₈₀1), and 2 wt % (S3Tw₈₀2): frequency-dependent oscillatory shear sweep (FIG. 8A), oscillatory amplitude sweep measured at a frequency of 10⁻¹ rad·s⁻¹ (FIG. 8B), storage modulus G′ (FIG. 8C) and tan (delta) (FIG. 8D) values measured at 10⁻¹ rad·s⁻¹, relaxation time (FIG. 5E), and yield stress (FIG. 8F).

FIG. 8G is a plot of extensional strain data of hydrogel compositions comprising Sangelose and varying concentrations of Tween 80.

FIG. 8H are representative images of hydrogel compositions comprising Sangelose and varying concentrations of Tween 80 under strain at a strain rate of 0.3 s⁻¹.

FIGS. 9A-9E are plots showing rheological measurements of Sangelose-Tween 20 hydrogel compositions with 3 wt % Sangelose and increasing concentrations of Tween 20 (0.1 wt % (S3Tw₂₀0.1), 0.75 wt % (S3Tw₂₀0.75), 1 wt % (S3Tw₂₀1), and 2 wt % (S3Tw₂₀2)): frequency-dependent oscillatory shear sweep (FIG. 9A), oscillatory amplitude sweep measured at a frequency of 10⁻¹ rad·s⁻¹ (FIG. 9B), storage modulus G′ (FIG. 9C) and tan (delta) (FIG. 9D) values measured at 10⁻¹ rad·s⁻¹, and yield stress (FIG. 9E).

FIGS. 9F and 9G are plots of rheological data on a Sangelose-Tween 20 hydrogel composition (S3Tw₂₀0.75) and a Sangelose only hydrogel (S3), showing shear-dependent viscosity (FIG. 9F) and step-shear measurements taken over three cycles of alternating low shear and high shear rates (FIG. 9G).

FIG. 10A is a schematic illustration of a timeline of mouse immunizations and blood collection.

FIGS. 10B and 10C are plots of anti-gE IgG titers measured at 4 and 8 weeks after administration of a shingles vaccine to mice, and area under curve (AUC) of anti-gE titer data generated from Sangelose-only vaccine hydrogel compositions with MPL as the adjuvant (FIG. 10B) or Sangelose-Tween 20 vaccine hydrogel compositions with different adjuvants (FIG. 10C).

FIG. 10D is plots of anti-gE IgG titers at 4 and 8 weeks after administration of a shingles vaccine to mice, and area under curve (AUC) of anti-gE titer data. The shingles vaccine was in a Sangelose hydrogel having varying concentrations of Tween 20.

FIG. 11A is a plot showing frequency-dependent oscillatory shear sweep rheological characterization of Sangelose hydrogel formulations (SANG-2-0.6) with and without semaglutide cargo (1.8 mg/mL).

FIG. 11B is a plot showing frequency-dependent oscillatory shear sweep rheological characterization of Sangelose hydrogel formulations (SANG-2-0.6) with and without tirzepatide cargo (4.5 mg/mL).

FIG. 11C is a plot showing frequency-dependent oscillatory shear sweep rheological characterization of Sangelose hydrogel formulations (SANG-2-0.6) with and without liraglutide cargo (1.8 mg/mL).

FIG. 12A is a plot of the % cumulative in vitro release of semaglutide over the course of one week from Sangelose-Tween 20 hydrogel compositions.

FIG. 12B is a plot of the % cumulative in vitro release of liraglutide over the course of one week from Sangelose-Tween 20 hydrogel compositions.

FIG. 13 is a schematic illustration of a timeline of semaglutide administration to diabetic rats and blood collection. Glucose measurements and serum collection were taken 5 days before injection of semaglutide and continued over a period of 6 weeks from injection.

FIG. 14 is a plot of data from an oral glucose tolerance test in rats, showing the area under the curve (AUC) of blood glucose after administration of 1) semaglutide as a bolus injection, 2) semaglutide in a Sangelose hydrogel composition, or 3) PBS.

FIG. 15 is a plot of the change in blood glucose (BG) of diabetic rats 5 days after administration of 1) semaglutide as bolus injection, 2) semaglutide in a Sangelose hydrogel composition, or 3) PBS.

FIG. 16 is a plot of the change in weight of diabetic rats 5 days after administration of 1) semaglutide as a bolus injection, 2) semaglutide in a Sangelose hydrogel composition, or 3) PBS.

FIG. 17 is a plot of semaglutide serum concentration in diabetic rats over the course of the first 5 days post administration of a daily bolus injection of semaglutide or a semaglutide-loaded Sangelose hydrogel composition.

FIG. 18 is a schematic illustration of tendon repair with and without the application of a hydrogel composition as described herein.

FIGS. 19A and 19B are schematic illustrations (FIG. 19A) and representative images (FIG. 19B) showing the interaction of a Sangelose hydrogel composition with a rheometer plate.

FIGS. 19C and 19D are plots of rheological data showing yield behavior (FIG. 19C) and yield stress (FIG. 19D) of Sangelose hydrogel compositions as measured on rheometer plates or on skin.

FIGS. 20A and 20B are representative images of a custom mechanical testing rig loaded with a cadaver arm for glide testing after repair of tendons and application of a hydrogel composition in the cadaver digits.

FIGS. 20C and 20D are plots of data from mechanical force measurements and show force traces from digits tested at 10 mm s⁻¹ (FIG. 20C) and initial peak load values for digits treated with nothing or with a Sangelose hydrogel composition (FIG. 20D.)

FIGS. 21A and 21B are side-view schematic illustrations of the printing of a filament while changing the speed of the nozzle and the flow rate independently, showing filament extension at a higher speed and a lower flow rate.

FIG. 21C is an overhead schematic illustration of a printed filament at varying flow rates.

FIG. 21D are representative images of printed Sangelose hydrogel compositions showing an increased extensional strain to break the filament as a function of Tween 80 concentration.

FIGS. 21E and 21F are plots of data relating to 3D printing of hydrogel compositions comprising Sangelose and various concentrations of Tween 80. FIG. 21E shows printing success as a function of flow rate. FIG. 21F shows the minimum filament diameter before disconnection is measured.

### DETAILED DESCRIPTION

The present technology provides compositions that are useful in a wide variety of applications, such as delivery of therapeutic agents, prevention of adhesions, and 3D printing. In some embodiments, the present disclosure provides a hydrogel composition, comprising a first agent, wherein the first agent is octadecyl modified hydroxypropyl methylcellulose (HPMC-C18), and one or more second agents comprising a surfactant or cyclic polysaccharide, wherein the hydrogel composition exhibits shear-thinning, self-healing, and dynamic viscoelastic properties, and optionally one or more therapeutic agents. The one or more second agents may non-covalently interact with the hydrophobic chains of the first agent. For instance, the second agent can have reversible, non-covalent interactions with a hydrophobic chain of the first agent. In some embodiments, the first agent forms micellar structures, and the one or more second agents disrupt the micellar structures.

In some embodiments, the present disclosure provides for a method of treating or preventing a disease or condition in an individual in need thereof, comprising administering to the individual in need thereof a hydrogel composition described herein.

In some embodiments, the present disclosure provides for a composition for use in treating or preventing a disease or condition in an individual in need thereof, wherein the composition for use is a hydrogel composition described herein.

In some embodiments, the present disclosure provides for a method for delivering a therapeutic agent to an individual in need thereof, comprising injecting a hydrogel composition described herein into the individual in need thereof.

In some embodiments, the present disclosure provides for a hydrogel composition described herein for use in 3D printing.

In some embodiments, the present disclosure provides for a method of forming an object from a hydrogel composition described herein using a 3D printing process.

The embodiments of the present disclosure can provide numerous advantages compared to conventional therapeutic products and treatment approaches. For instance, conventional hydrogel-based depot technologies typically exhibit several critical shortcomings, including complicated manufacturing, poor formulation stability, challenging administration, burst release that can contribute to poor tolerability of the therapy, and insufficiently slow release to enable appropriately long-acting therapies. In contrast to conventional covalently crosslinked hydrogels, the hydrogel compositions of the present technology are formed through strong yet dynamic physical interactions. As a result, these materials can address the shortcomings of other hydrogel-based depot technologies by exhibiting: (i) mild formulation requirements favorable for facile formulation with therapeutic agents such as incretin mimetics and vaccines, and maintaining drug stability during manufacturing and storage; (ii) shear-thinning properties allowing for straightforward injectability through standard syringes and needles, thus improving patient convenience; (iii) rapid self-healing of hydrogel structure and depot formation to avoid burst release of the drug cargo, thus providing excellent tolerability by maintaining consistent slow release to circumvent undesirable side effects (e.g., gastrointestinal discomfort); (iv) sufficiently high yield stress to form a robust depot that persists under the normal stresses of the subcutaneous space following administration; (v) prolonged delivery of the therapeutic agent allowing for continuous delivery over clinically desirable timeframes; (vi) biodegradability; and/or (vii) non-immunogenicity, as well as not promoting immune responses to the encapsulated therapeutic agent.

In some embodiments, the present disclosure provides materials with characteristics that are particularly advantageous for achieving sustained drug delivery. According to embodiments of the present technology, a potent hydrogel composition is provided for slow drug delivery. Compositions including Sangelose combined with Tween 20 or αCD can create a physically crosslinked hydrogel with shear-thinning and self-healing properties with high enough yield stress to form a depot subcutaneously and serve as a drug delivering niche under the skin. The compositions disclosed herein can outperform other hydrogel-based compositions in that the preparation steps are extremely easy and quick-simple mixing of Sangelose and Tween 20 or αCD forms readily available hydrogel for injection. The mechanical properties of the hydrogel can be easily changed by changing the concentration of the polymer and/or surfactant to fit different use purposes. Preparation methods based on simple mixing, rather than the complicated synthesis processes required by many conventional hydrogel compositions, can ensure that the properties of the hydrogel are uniform and can improve therapeutic performance.

In some embodiments, the present disclosure provides an extensible material that can stretch under high printing speed and that can be easily injected through the printing nozzle. For instance, compositions including Sangelose with Tween 80 can exhibit the distinctive feature of having high extensibility with low yield stress. The strain to break under extensional rheology may further improve with increased concentrations of Tween 80. Such highly extensible materials, which can be easily fabricated and tuned as described herein, can provide a good alternative for conventional 3D printing materials to improve printing speed, among other advantages.

Embodiments of the present disclosure will be described more fully hereinafter with reference to the accompanying drawings in which example embodiments are shown. Embodiments of the claims may, however, be embodied in many different forms and should not be construed as limited to the embodiments set forth herein. The examples set forth herein are non-limiting examples and are merely examples among other possible examples.

The headings provided herein are for convenience only and do not interpret the scope or meaning of the claimed present technology. Embodiments under any one heading may be used in conjunction with embodiments under any other heading.

### I. Hydrogel Compositions

The present technology utilizes hydrogel compositions that can serve as a versatile platform for various applications, including: controlled release of a therapeutic agent, such as those described in Section I.B below; methods for treatment of individuals, such as those described in Section III below; and methods for 3D printing, such as those described in Section IV below. In some embodiments, the hydrogel compositions exhibit dynamic behavior, such as shear-thinning behavior, self-healing behavior, and/or highly tunable viscoelastic mechanical properties, such as the properties described in Section II below. The shear-thinning, self-healing, and/or viscoelastic properties of the hydrogel compositions can result from reversible, non-covalent, supramolecular interactions between the components of the hydrogel (e.g., the first and second agents, as described further below). The non-covalent interactions can include physical crosslinking, which may encompass various types of crosslinking arising from weak physical interactions such as hydrogen bonding, hydrophobic interactions, ionic interactions, van der Waals interactions, host-guest interactions, crystal formation, physical entanglement, or combinations thereof. The non-covalent interactions can allow for the formation of dynamic, reversible crosslinks between components of the hydrogel that are capable of dissociating and reforming, e.g., spontaneously and/or in response to applied stress.

The hydrogel compositions described herein comprise a first agent, wherein the first agent is a hydrophobically modified polysaccharide; one or more second agents, wherein the second agent has a high hydrophilic/hydrophobic ratio and/or non-covalently interacts with hydrophobic chains; and optionally one or more therapeutic agents. The non-covalent interactions between the hydrophobically modified polysaccharide (e.g., HPMC-C18) and the second agent influences the dynamic character of the hydrogel.

For instance, in the polymer hydroxypropyl methylcellulose (HPMC) modified with long hydrocarbon side chains, hydrophobic interactions between the hydrocarbon chains may result in areas of side chain packing and/or organized assembly, resulting in a hydrogel having a relatively static character rather than a hydrogel with dynamic properties. The hydrophobic side chains may form micelles, aggregates of hydrophobic hydrocarbon chains surrounded by the hydrophilic backbone of HPMC, contributing to a relatively static character. Introduction of a second agent capable of interacting with the hydrocarbon side chains may disrupt the organized interactions of hydrocarbon side chains and form free volume (e.g., cavities) within the hydrocarbon chain packed regions of the micelles, thus producing a hydrogel with desirable dynamic properties.

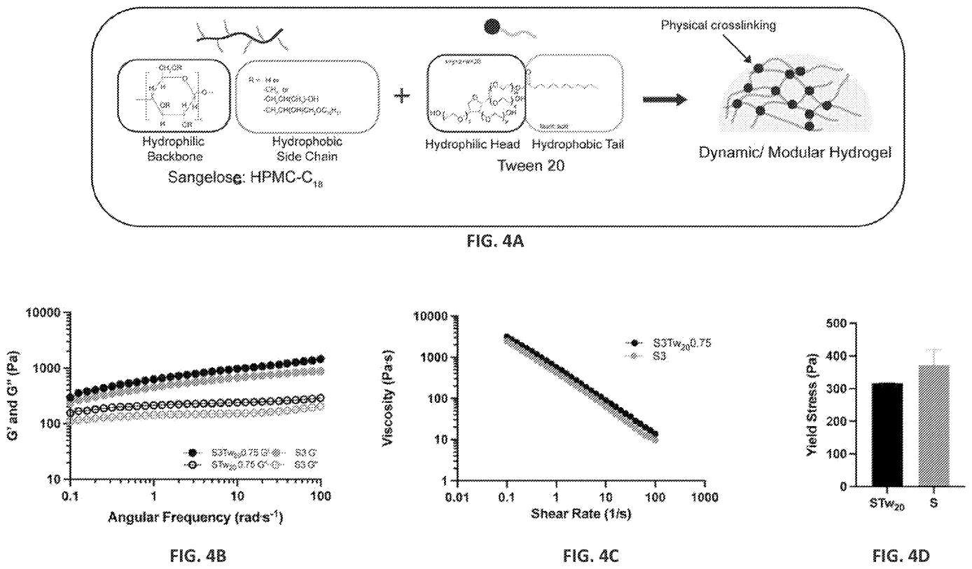

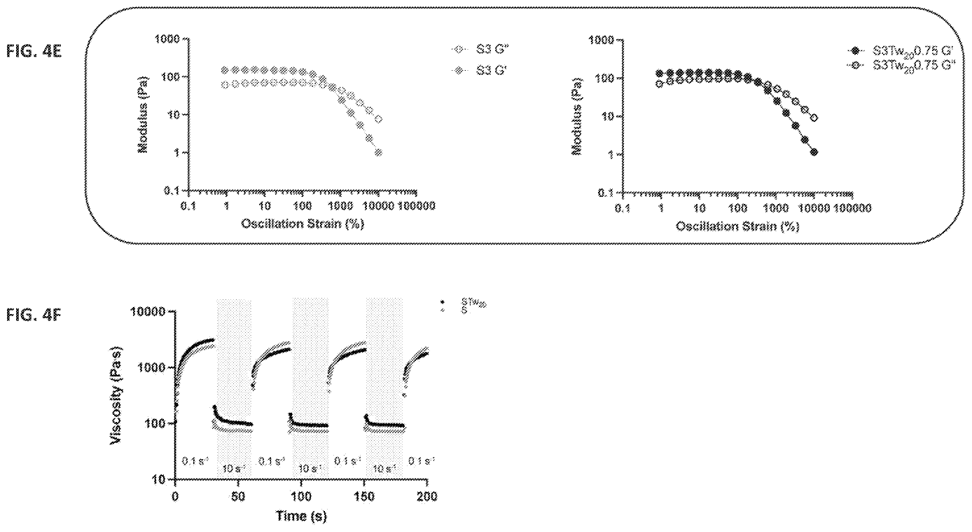

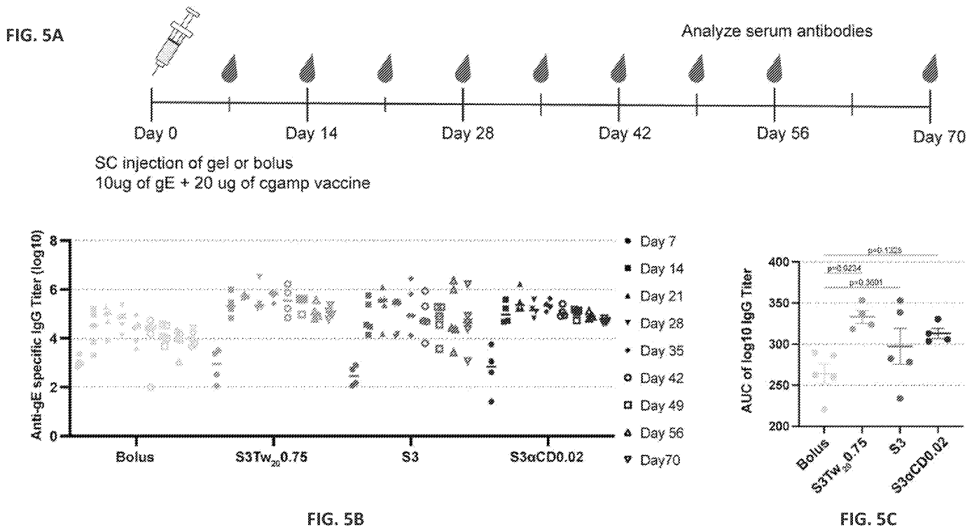

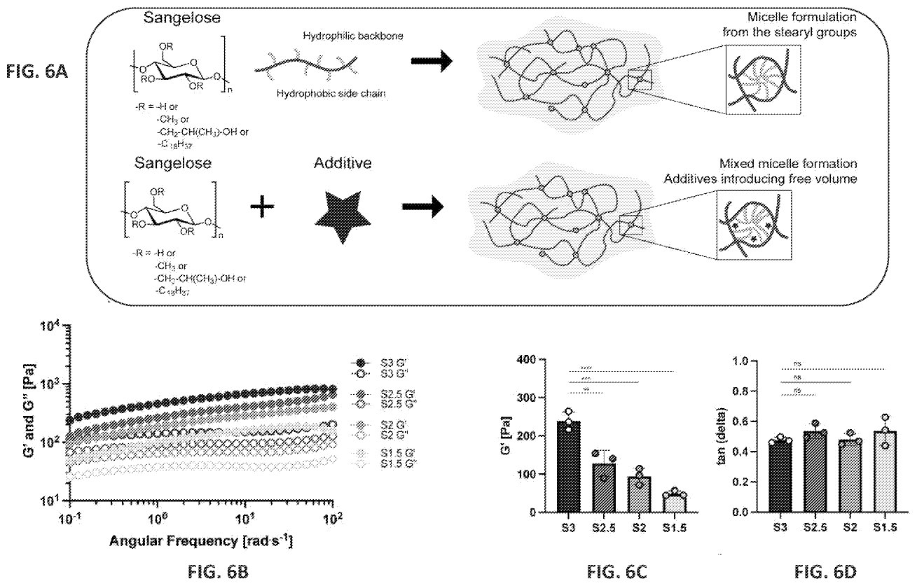

FIG. 6A provides schematic illustration of hydrogels formed from Sangelose, an HPMC modified with a C18 hydrocarbon (top), and hydrogels formed from Sangelose and a second agent (bottom). Sangelose has a hydrophilic cellulose backbone from which hydrophobic C18 side chains are attached. The hydrophobic side chains of Sangelose interact with each other forming hydrophobic aggregates surrounded by the hydrophilic backbone of the cellulose, in what can be described as a micelle. Micelle formation may occur along a single polymer chain and/or between different polymer chains to form crosslinks. When enough interactions between different polymer chains are formed, a hydrogel will form. The resulting hydrogel composition can have a relatively static character (e.g., the crossover frequency of the storage modulus (G′) and the loss modulus (G″) of the hydrogel composition can be less than 0.01 rad·s⁻¹).

To form a hydrogel composition with a dynamic character, a second agent is added to modify the network dynamics of the Sangelose polymer chains. In some embodiments, addition of a second agent (e.g., a surfactant), represented by the star in FIG. 6A, at least partially disrupts the hydrophobic interactions within the micelle to form a “mixed micelle.” For instance, the second agent can include a hydrophobic chain having a length that is different than the length of the Sangelose side chains, can include an unsaturated hydrocarbon group that disrupts alignment between neighboring chains, and/or can include sterically bulky moieties (e.g., large hydrophilic headgroups) that reduce packing density. Unsaturated hydrocarbon groups contain alkenes (—CH═CH—) or alkynes (—C≡C—) in their structure. For instance, oleyl and linoleyl are mono- and di-unsaturated hydrocarbon groups, respectively. In such embodiments, the second agent may be dispersed within the micelle to create free volume (e.g., cavities) within the micelle.

Alternatively or in combination, addition of a second agent (e.g., alpha-cyclodextrin) can at least partially disrupt the micelle by binding to the hydrophobic side chains of Sangelose, thereby preventing or at least hindering the hydrophobic side chains from entering the micelles and creating free volume within the micelles. In such embodiments, the second agent may be dispersed within the micelles, may be located only partially within the micelles, or may be located entirely outside of the micelles.

The addition of the second agent modifies the micellar character, promotes reversible crosslinking of the polymer, increases the dynamic nature, and/or reduces the static nature of the hydrogel composition (e.g., the crossover frequency of the storage modulus (G′) and the loss modulus (G″) of the hydrogel composition can be greater than 0.01 rad·s⁻¹). The increased dynamic nature of the hydrogel may permit, for instance, penetration of immune cells into the hydrogel, while retaining therapeutic agents in the hydrogel, as compared to hydrophobically modified HPMC in the absence of a second agent. Additional examples of first and second agents suitable for use in the hydrogel compositions herein are provided in Sections I.A.1 and I.A.2 below, respectively.

The hydrogel compositions described herein can provide many advantages for therapeutic applications. For instance, the hydrogel compositions described herein can exhibit high drug loading capacity, gentle conditions for encapsulation of biologic cargo, sustained delivery of cargo, and/or mechanical tunability. However, unlike traditional covalently crosslinked hydrogels, the hydrogel compositions herein can be easily administered via techniques such as direct injection, catheter delivery, spreading, or spraying, due to their shear-thinning and/or self-healing properties. Additionally, the hydrogel compositions herein can exhibit unique dynamic network rearrangements that provide highly tunable release characteristics for the therapeutic cargo. The hydrogel compositions provided herein can also be synthesized in a straight-forward, cost-effective manner that is easily scalable.

### A. Hydrogel Components

### 1. First Agent—Hydrophobically Modified Polysaccharide

The first agent refers to a hydrophobically modified polysaccharide. Examples of polysaccharides include cellulose, alginate, chitosan, hyaluronic acid, starch, agarose, agar, and xanthan gum. In some embodiments, the polysaccharide is a derivative of a naturally occurring polymer, such as a cellulose derivative. Examples of cellulose derivatives include hydroxypropyl methylcellulose (HPMC), hydroxyethyl cellulose (HEC), hydroxypropyl cellulose (HPC), ethylcellulose (EC), methylcellulose (MC), hydroxyethyl methylcellulose (HEMC), carboxymethylcellulose (CMC), carboxymethyl ethyl cellulose (CMEC), and combinations thereof. In some embodiments, the polysaccharide is HPMC.

Hydrophobic modification of the polysaccharide provides a hydrophilic polymer backbone with hydrophobic side chains. The hydrophobic side chain can be or include a hydrophobic group having a plurality of carbon atoms (e.g., from 2 to 50 carbon atoms, 2 to 30 carbon atoms, or 2 to 18 carbon atoms), and can be a saturated molecule or an unsaturated molecule. Examples of hydrophobic groups that may be used include, but are not limited to, alkyl groups (e.g., C4 to C18 alkyls, such as butyl (—C4), hexyl (—C6), octyl (—C8), decyl (—C10), dodecyl (—C12), tetradecyl (—C14), pentadecyl (—C15), hexadecyl (—C16), heptadecyl (—C17), octadecyl (—C18), alkenyl groups (e.g., oleyl, linoleyl), aryl groups (e.g., phenyl, benzyl, pyryl, naphthyl, anthracene), and cycloalkyl groups (e.g., adamantyl, cyclohexyl, cholesterol). In some embodiments, the degree of modification of the polysaccharide (e.g., percentage of reactive groups on the polysaccharide have been functionalized with the hydrophobic side chain) is within a range from 0.1% to 5%, 0.1% to 1%, 0.1% to 3%, 1% to 50%, 5% to 30%, 5% to 25%, or 10% to 15%. For example, the degree of modification can be about 5%, 10%, 15%, 20%, or 25%.

In some embodiments, the hydrophobically modified polysaccharide is hydrophobically modified HPMC. In some embodiments, the hydrophobically modified polysaccharide has a plurality of hydrocarbon groups, each having 14 to 22 carbon atoms. In some embodiments, the first agent is modified HPMC having a plurality of hydrocarbon groups, each having 14 to 22 carbon atoms. In some embodiments, the hydrophobically modified polysaccharide is octadecyl modified HPMC (HPMC-C18).

Sangelose is a tradename of HPMC-C18, of which the chemical formula is shown below:

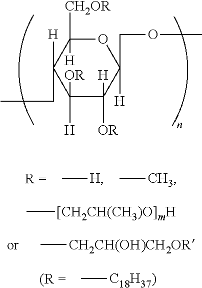

The hydrophobic side chains of the hydrophobically modified polysaccharide can interact noncovalently with each other, thereby forming noncovalent crosslinks between different polysaccharide chains to form a hydrogel. For instance, the hydrophobic side chains can aggregate with each other to form micellar structures that serve as the noncovalent crosslinks of the hydrogel. These crosslinks can be relatively static (e.g., due to tight packing of the chains), such that hydrogels formed from the hydrophobically modified polysaccharide only are static rather than dynamic hydrogels.

### 2. Second Agent

The second agent can be an additive that is capable of non-covalently interacting with the hydrophobic side chains of the first agent, such that the interaction disrupts the hydrophobic side chain packing or organization, thereby increasing the dynamic character of the hydrogel crosslinks to produce a dynamic hydrogel. For instance, the second agent can be dispersed within and/or disrupt the micellar structures of the first agent, thereby creating free volume within the micellar structures. Alternatively or in combination, the second agent can bind non-covalently to the hydrophobic side chains of the first agent to prevent the hydrophobic side chains from interacting with other hydrophobic side chains within the micellar structure. In some embodiments, the second agent is a surfactant or a cyclic polysaccharide.

In some embodiments, the second agent is a surfactant. Examples of surfactants include nonionic surfactants, anionic surfactants, cationic surfactants, and/or zwitterionic surfactants. A surfactant can include at least one hydrophobic tail and a hydrophilic head group. The hydrophobic tail can be or include a hydrophobic group having a plurality of carbon atoms (e.g., from 2 to 50 carbon atoms, 2 to 30 carbon atoms, or 2 to 18 carbon atoms), and can be a saturated group or an unsaturated group, and can be a branched group or an unbranched molecule. Examples of hydrophobic groups that may be used include, but are not limited to, alkyl groups (e.g., C4 to C18 alkyls, such as butyl (—C4), hexyl (—C6), octyl (—C8), decyl (—C10), dodecyl (—C12), tetradecyl (—C14), pentadecyl (—C15), hexadecyl (—C16), heptadecyl (—C17), and octadecyl (—C18), alkenyl groups (e.g., oleyl, linoleyl), aryl groups (e.g., phenyl, benzyl, pyryl, naphthyl, anthracene), and cycloalkyl groups (e.g., adamantyl, cyclohexyl, cholesterol). In some embodiments, the length of the hydrophobic tail is selected to be different than (e.g., longer or shorter than) the length of the hydrophobic side chain of the first agent. In some embodiments, the hydrophobic tail is unsaturated and/or branched. In some embodiments, the surfactant includes a single hydrophobic tail, while in other embodiments, the surfactant includes multiple hydrophobic tails (e.g., two, three, or more). The hydrophilic head group can be a nonionic group, an anionic group, a cationic group, or a zwitterionic group. In some embodiments, relatively large hydrophilic head groups (e.g., polymers, polyethoxylated sorbitan) are advantageous for disrupting micellar structures due to steric hindrance.

In some embodiments the surfactant is a non-ionic surfactant. An example of a non-ionic surfactant is the family of polysorbates, which are derived from ethoxylated sorbitan esterified with fatty acids. For instance, polyoxyethylene (20) sorbitan monooleate is derived from polyethoxylated sorbitan and oleic acid. The number 20 following the ‘polyoxyethylene’ part refers to the total number of oxyethylene (—CH₂CH₂O—) groups found in the molecule. Polyoxyethylene (20) sorbitan monooleate is more commonly referred to as polysorbate 80 or Tween 80. The number following the ‘polysorbate’ part is related to the type of major fatty acid associated with the molecule, where monooleate is indicated by 80. The chemical formula of polyoxyethylene (20) sorbitan monooleate (Tween 80 or polysorbate 80) is:

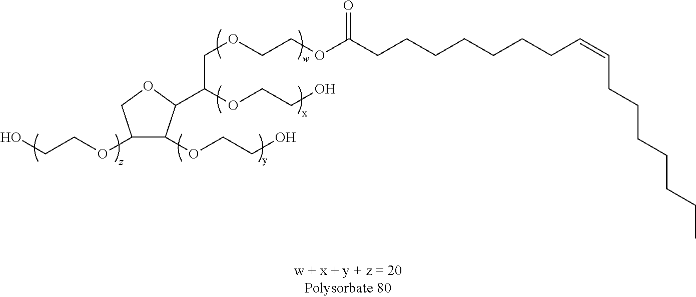

Other examples of polysorbate include polyoxyethylene (20) sorbitan monolaurate (polysorbate 20 or Tween 20), polyoxyethylene (20) sorbitan monopalmitate (polysorbate 40 or Tween 40), and polyoxyethylene (20) sorbitan monostearate (polysorbate 60 or Tween 60). Monolaurate is indicated by 20, monopalmitate is indicated by 40, monostearate by 60, and monooleate by 80.

In some embodiments, the second agent is a non-ionic surfactant (e.g., a polysorbate or a polyoxyethylene fatty ether). In some embodiments, the second agent is a polysorbate. In some embodiments the polysorbate is polyoxyethylene (20) sorbitan monooleate (Tween 80 or polysorbate 80), or polyoxyethylene (20) sorbitan monolaurate (polysorbate 20 or Tween 20).

Another example of a non-ionic surfactant is the family of polyoxyethylene fatty ethers, commonly referred to by their tradename Brij surfactants. Brij surfactants contain a polyethylene oxide polar head group and a hydrocarbon hydrophobic chain derived from a straight or branched hydrocarbon chain alcohol. Examples of alcohols used to prepare Brij surfactants include: oleyl alcohol (O, C18, one unsaturation), stearyl alcohol (S, C18, saturated), cetyl alcohol (C, C16, saturated), and lauryl alcohol (L, C12, saturated). Some Brij surfactants are derived from a mixture of fatty alcohols, for example cetyl alcohol and stearyl alcohol.

Brij surfactants have the general chemical formula:

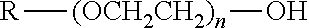

where R represents the hydrocarbon chain of the parent alcohol and n is the number of ethylene oxide units. In some embodiments, R may have a chain length of 12 to 18 carbons and/or n may be 2 to 100. Brij molecules as described herein may be referred to by the parent alcohol and the number of ethylene oxide units. For instance, Brij O10, refers to a Brij molecule derived from oleyl alcohol (O), and having 10 ethylene oxide units. Brij O20 refers to a Brij molecule derived from oleyl alcohol (O), and having 20 ethylene oxide units. Brij C20 refers to a Brij molecule derived from cetyl alcohol (C), and having 20 ethylene oxide units. In some embodiments, the second agent is a polyoxyethylene fatty ether.

Other examples of the second agent include poloxamers (e.g., P188, P237, P338, P407), alkyl sulfates (e.g., dodecyl sulfate) and salts thereof (e.g., sodium dodecyl sulfate (SDS)), fatty acids (e.g., lauric acid, myristic acid, palmitic acid, stearic acid), fatty alcohols (e.g., lauryl alcohol, myristyl alcohol, palmityl alcohol, stearyl alcohol), phospholipids, and other lipids or derivatives thereof exhibiting surfactant behavior.

The hydrophilic-lipophilic balance (HLB), often used to describe surfactants or emulsifiers, may be used to describe the amphiphilic character of a molecule and is mainly based on the respective sizes/structure of the hydrophobic and hydrophilic groups of the molecule. For instance, the size and length of the fatty acid or polyethylene oxide groups influence the amphiphilic character and corresponding HLB value. HLB is a measure of a molecule's degree of hydrophilicity or lipophilicity and is calculated from the weight percentage of the hydrophilic groups to the hydrophobic groups in a molecule, with values ranging from 1-20. Molecules with low HLB values are more oil-soluble (lipophilic), while those with higher values are more water-soluble (hydrophilic).

Table 1 lists the HLB values of non-limiting, exemplary Span, Tween, and Brij surfactants.

TABLE 1

| Exemplary surfactants | HLB |
|---|---|
| Span 20 | 8.6 |
| Span 40 | 6.7 |
| Span 80 | 4.3 |
| Tween 20 | 16.7 |
| Tween 40 | 15.6 |
| Tween 80 | 15.0 |
| Brij C2 | 5.3 |
| Brij C20 | 12.9 |
| Brij O5 | 9.1 |
| Brij O10 | 12.4 |
| Brij O20 | 15.3 |

In some embodiments, the second agent has a hydrophilic-lipophilic balance (HLB) value of at least 9, at least 10, at least 11, at least 12, at least 13, at least 14, or at least 15. In some embodiments, the HLB value is at least 9. In some embodiments, the HLB value is at least 14.

In some embodiments, the second agent is a non-ionic surfactant having a HLB value of at least 12, at least 13, or at least 14. In some embodiments, the second agent is a polysorbate having a HLB value of at least 12, at least 13, or at least 14. In some embodiments, the second agent is a Brij molecule having a HLB value of at least 10, at least 11, at least 12, at least 13, or at least 14.

In some embodiments, the second agent is a cyclic polysaccharide, and the cyclic polysaccharide is a cyclodextrin. As used herein, a cyclodextrin consists of a macrocyclic ring of glucose subunits joined by alpha-1,4 glycosidic bonds. Cyclodextrins have five or more glucose monomers linked in a ring forming the shape of a hollow truncated cone. The exterior of the cone is hydrophilic and the cavity of the cone is hydrophobic or lipophilic. This structure permits the cyclodextrin to be water soluble while permitting the ability to accommodate hydrophobic molecules/groups in the cavity; for instance, hydrophobic hydrocarbon chains of the first agent. An example of a cyclodextrin derived from 6 glucose subunits, an α-cyclodextrin (αCD) is depicted below:

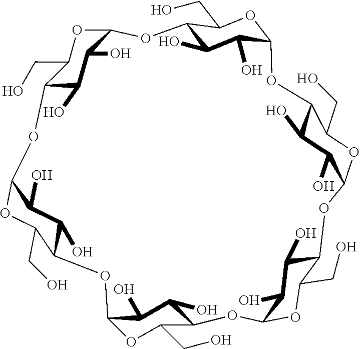 [SMILES: OCC1O[C@@H]2O[C@@H]3C(CO)O[C@H](O[C@@H]4C(CO)O[C@H](O[C@@H]5C(CO)O[C@H](O[C@@H]6C(CO)O[C@H](O[C@@H]7C(CO)O[C@H](O[C@H]1[C@H](O)[C@H]2O)[C@H](O)[C@H]7O)[C@H](O)[C@H]6O)[C@H](O)[C@H]5O)[C@H](O)[C@H]4O)[C@H](O)[C@H]3O]

Examples of cyclodextrins include but are not limited to α-cyclodextrin (αCD, 6 glucose monomers), β-cyclodextrin (BCD, 7 glucose monomers), and γ-cyclodextrin (γCD, 8 glucose monomers). In some embodiments, the second agent is water soluble while having some affinity for hydrophobic groups such as hydrocarbon side chains of the first agent. Such affinity permits interaction between the first and second agent. For instance, the second agent may physically disrupt hydrocarbon chain packing. In some embodiments, the cyclic polysaccharide comprises a ring of five to ten glucose monomers. In some embodiments, the cyclic polysaccharide comprises a ring of five to eight glucose monomers. In some embodiments, the cyclic polysaccharide comprises a ring of five to six glucose monomers. In some embodiments, the cyclic polysaccharide comprises a ring of six glucose monomers. In some embodiments, the cyclic polysaccharide is αCD. In some embodiments, the cyclic polysaccharide is BCD. In some embodiments, the cyclic polysaccharide is γCD.

In some embodiments, the second agent has a hydrophilic-lipophilic balance (HLB) value of at least 9, at least 10, at least 11, at least 12, at least 13, at least 14, or at least 15, and interacts with the hydrophobic chains of the first agent and at least partially disrupts the hydrophobic chain interactions. In some embodiments the second agent forms a mixed micelle with the first agent. In some embodiments, the first agent forms micellar structures and the second agent is dispersed within the micellar structures to form at least one mixed micelle. In some embodiments, the second agent non-covalently interacts with the hydrophobic side chains of the first agent (e.g., HPMC-C18). In some embodiments, the non-covalent interaction disrupts organization or packing of the hydrophobic side chains of the first agent.

In some embodiments, the first agent is a hydrophobically modified polysaccharide (e.g., HPMC-C18); and the second agent is a surfactant (e.g., Tween 20 or Tween 80) or a cyclic polysaccharide (e.g., cyclodextrin).

### B. Therapeutic Agents

In some embodiments, the present disclosure provides for hydrogel compositions that encapsulate and deliver a therapeutic agent for preventing or treating a disease or condition in an individual. The therapeutic agent can include small molecule drugs, peptides (e.g., acylated peptides), proteins (e.g., antibodies, cytokines, antigens), nucleic acids (e.g., mRNA, DNA, siRNA—which may be naked molecules, combined with a delivery vector, or incorporated into a viral vector), polysaccharides, cells, and/or suitable combinations thereof. The therapeutic agent can be encapsulated in the hydrogel via physical entrapment (e.g., if the size of the therapeutic agent is larger than the mesh size of hydrogel), interaction with hydrogel components (e.g., hydrophobic interactions with the hydrophobic side chains of the first agent that form micelle crosslinks of the hydrogel), or suitable combinations thereof. In some embodiments, the therapeutic agent is an incretin mimetic, an acylated peptide, or a vaccine.

### 1. Incretin Mimetics

In some embodiments, the therapeutic agent is an incretin mimetic. Incretin mimetics are compounds that mimic the activity of incretin hormones. Incretin hormones are peptides that are secreted by the gut in response to nutrient ingestion. The primary incretin hormones are glucagon-like peptide 1 (GLP-1) and gastric inhibitory peptide (GIP). GLP-1 contributes to the regulation of glucose homeostasis within the body through its interaction with the GLP-1 receptor. GLP-1 is secreted from intestinal L-cells in response to nutrients and lowers blood glucose by stimulating insulin and suppressing glucagon secretion in a glucose-dependent manner, reducing the risk of hypoglycemia. In addition, GLP-1 is also a neurotransmitter synthesized by preproglucagon neurons in the brain and acts via central pathways to lower energy intake through an effect on satiety, hunger, and reward-related measures, leading to a lowering of body weight. Metabolic effects of GLP-1 include glucose-dependent stimulation of insulin secretion, inhibition of glucagon secretion, inhibition of food intake, decrease of gastric emptying, and increase of natriuresis and diuresis. GLP-1 has also been shown to influence learning, memory, reward behavior, and palatability, as well as to exhibit neuroprotective, cardioprotective, and anti-inflammatory effects. However, the therapeutic applicability of native GLP-1 is limited by its short half-life in vivo (approximately 2 to 3 minutes) and inactivation by the enzyme dipeptidyl peptidase 4 (DPP4). GIP is secreted by enteroendocrine K-cells in response to nutrients and also exhibits an insulinotropic effect via binding to the GIP receptor. However, unlike GLP-1, GIP stimulates glucagon secretion. GIP also influences appetite, fat accumulation, memory, and bone formation. Native GIP also exhibits a short half-life (approximately 4 to 5 minutes) and is inactivated by DPP4.

In some embodiments, the incretin mimetic is a GLP-1 receptor agonist (RA). GLP-1 RAs (also known as “GLP-1 analogues”) are a class of drugs that interact with the GLP-1 receptor and display structural similarities to native GLP-1, but with modifications to extend the in vivo half-life and thus provide improved bioavailability. GLP-1 RAs can be categorized as either short-acting or long-acting compounds. Short-acting GLP-1 RAs have been rendered resistant to cleavage by DPP4 by altering the amino acids at the second and third N-terminal positions, but are still subject to renal elimination and thus generally have a half-life from approximately 2 to 5 hours. Examples of short-acting GLP-1 RAs include exenatide and lixisenatide. Long-acting GLP-1 RAs implement mechanisms to reduce renal elimination, such as acylation with fatty acids to facilitate binding to serum albumin or conjugation to a larger molecule/component, and thus can have a half-life from 12 hours to several days. Examples of long-acting GLP-1 RAs include liraglutide (acylation with C16 fatty monoacid), semaglutide (acylation with C18 fatty diacid), tirzepatide (acylation with C20 fatty diacid), retatrutide (acylation with C20 fatty diacid), albiglutide (conjugation to albumin), dulaglutide (conjugation to Fc fragment of IgG), and exenatide-LAR (long-acting release) (coupled to biodegradable polymer microspheres).

The GLP-1 RA can include a peptide that binds to the GLP-1 receptor. In some embodiments, the peptide can be an analogue of a native GLP-1 peptide, such as the endogenous human GLP-1 peptide (e.g., GLP-1 (7-36) or GLP-1 (7-37)). For example, the peptide of the GLP-1 RA can include a sequence having at least 50%, 60%, 70%, 75%, 80%, 85%, 90%, 95%, 97%, 99%, or 100% sequence identity to a native GLP-1 peptide. The peptide can be produced in suitable host cells via recombinant DNA technology, can be produced in a cell-free system, or can be produced synthetically via solid phase synthesis.

In some embodiments, the GLP-1 RA is a mono-receptor agonist that binds exclusively to the GLP-1 receptor. Examples of GLP-1 mono-receptor agonists include exenatide, exenatide-LAR, lixisenatide, liraglutide, semaglutide, albiglutide, dulaglutide, efpeglenatide, and ecnoglutide. The peptide of the GLP-1 mono-receptor agonist can be an analogue of a native GLP-1 peptide, as previously described.

In some embodiments, the GLP-1 RA is a dual-receptor agonist that binds to the GLP-1 receptor and an additional receptor. The additional receptor can be any of the following receptors: a glucagon receptor, a GIP receptor, a cholecystokinin receptor, a xenin receptor, a secretin receptor, a neuropeptide Y receptor, or a neurotensin receptor. For instance, the GLP-1 RA can be a dual GLP-1/glucagon receptor agonist that binds to the GLP-1 receptor and the glucagon receptor. Examples of dual GLP-1/glucagon receptor agonists include efinopegdutide, cotadutide, mazdutide, and BI 45690. In such embodiments, the peptide of the dual GLP-1/glucagon receptor agonist can be an analogue of oxyntomodulin, which is a gut hormone that activates both the GLP-1 receptor and the glucagon receptor. As another example, the GLP-1 RA can be a dual GLP-1/GIP receptor agonist that binds to the GLP-1 receptor and the GIP receptor. Examples of dual GLP-1/GIP receptor agonists include tirzepatide, LY3493269, VK2735, CT-868, and AMG133. In such embodiments, the peptide of the dual GLP-1/GIP receptor agonist can be an analogue of GIP, which has high sequence similarity to GLP-1 in the N-terminal part of the peptide.

In some embodiments, the GLP-1 RA is a triple-receptor agonist that binds to the GLP-1 receptor and two additional receptors. The additional receptors can be any two of the following receptors: a glucagon receptor, a GIP receptor, a cholecystokinin receptor, a xenin receptor, a secretin receptor, a neuropeptide Y receptor, or a neurotensin receptor. For example, the GLP-1RA can be a triple GLP-1/glucagon/GIP receptor agonist that binds to the GLP-1 receptor, the glucagon receptor, and the GIP receptor. Examples of triple GLP-1/glucagon/GIP receptor agonists include retatrutide.

In some embodiments, the peptide of the GLP-1 RA is attached to at least one substituent (also referred to herein as a “side chain”). The substituent can prolong the half-life of the peptide in vivo, such as by binding of the substituent to serum albumin. Optionally, the substituent can provide other beneficial effects, such as enhancing solubility, promoting cellular uptake, and/or reducing immunogenicity. The substituent of the GLP-1 RA can be attached to the peptide of the GLP-1 RA via any suitable mechanism, such as acylation, alkylation, ester formation, amide formation, coupling to a cysteine residue, and/or other conjugation chemistries known to those of skill in the art. For example, the substituent can be covalently attached to the peptide via an amide bond between a carboxyl group of the substituent and an amino group of the peptide. The amino group of the peptide can be the N-terminal amino group of the peptide or can be a side chain amino group of an amino acid residue of the peptide (e.g., an amino group of a lysine residue of the peptide). The substituent can be attached to the peptide directly, or can be attached to the peptide indirectly via a linker, which may also be referred to herein as a spacer. For instance, the substituent can be attached to the peptide via an amide bond between a carboxyl group of the linker and an amino group of an amino acid residue of the peptide. The linker can be any suitable linker known to those of skill in the art, such as a peptide linker (e.g., a γ-glutamate linker), a hydrophilic spacer (e.g., 8-amino-3,6-dioxaoctanoic acid), a hydrophobic spacer, or combination thereof.

For example, the GLP-1 RA can be an acylated peptide that is attached to a lipophilic substituent having a plurality of carbon atoms, such as at least 10, 15, 20, 25, 30, 35, or 40 carbon atoms. In some embodiments, the lipophilic substituent is an acyl group of a fatty acid, such as a straight chain fatty acid or a branched fatty acid. The fatty acid can be a fatty monoacid, e.g., an aliphatic monocarboxylic acid having 4 to 38 carbon atoms, which may be saturated or unsaturated. The fatty acid can be a fatty diacid, e.g., an aliphatic dicarboxylic acid having 4 to 38 carbon atoms, which may be saturated or unsaturated. The fatty acid can be a C4 to C38 fatty acid, such as a C4 fatty acid, a C6 fatty acid, a C8 fatty acid, a C10 fatty acid, a C12 fatty acid, a C14 fatty acid, a C15 fatty acid, a C16 fatty acid, a C17 fatty acid, a C18 fatty acid, a C20 fatty acid, a C22 fatty acid, a C24 fatty acid, a C26 fatty acid, a C28 fatty acid, a C30 fatty acid, a C32 fatty acid, a C34 fatty acid, a C36 fatty acid, or a C38 fatty acid.

In some embodiments, the lipophilic substituent is an acyl group having the formula CH₃(CH₂)ₙCO—, where n is an integer from 4 to 38, or from 4 to 24, such as CH₃(CH₂)₄CO—, CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO—, CH₃(CH₂)₂₂CO—, or CH₃(CH₂)₂₄CO—.

In some embodiments, the lipophilic substituent is an acyl group having the formula HOOC(CH₂)ₙCO—, where n is an integer from 4 to 38, or from 4 to 24, such as HOOC(CH₂)₁₄CO—, HOOC(CH₂)₁₆CO—, HOOC(CH₂)₁₈CO—, HOOC(CH₂)₂₀CO—, or HOOC(CH₂)₂₂CO—.

In some embodiments, the lipophilic substituent is an acyl group of a straight-chain or branched alkane α,ω-dicarboxylic acid.

In some embodiments, the lipophilic substituent is an acyl group having the formula HOOC(CH₂)ₙCO—, where n is an integer from 4 to 38, or from 4 to 24, such as HOOC(CH₂)₁₆CO—, HOOC(CH₂)₁₈CO—, HOOC(CH₂)₂₀CO—, or HOOC(CH₂)₂₂CO.

In some embodiments, the lipophilic substituent is an acyl group having the formula CH₃(CH₂)ₙCO—NHCH(COOH)(CH₂)₂CO—, where n is an integer from 10 to 24.

In some embodiments, the lipophilic substituent is an acyl group having the formula CH₃(CH₂)ₙCO—NHCH((CH₂)₂COOH)CO—, where n is an integer from 8 to 24.

In some embodiments, the lipophilic substituent is an acyl group having the formula COOH(CH₂)ₙCO—, where n is an integer from 8 to 24.

In some embodiments, the lipophilic substituent is an acyl group having the formula —NHCH(COOH)(CH₂)₄NH—CO(CH₂)ₙCH₃, where n is an integer from 8 to 18.

The lipophilic substituent can be attached to the peptide of the GLP-1 RA via a linker, as described herein. In some embodiments, the lipophilic substituent interacts with a component of the dynamic hydrogel to promote encapsulation and controlled release of the GLP-1 RA from the dynamic hydrogels, as described herein. For instance, in embodiments where the dynamic hydrogel includes a hydrophobically modified polysaccharide (e.g., HPMC-C18), the lipophilic substituent can interact with hydrophobic sidechains of the hydrophobically modified polysaccharide, thereby causing the GLP-1 RA to be encapsulated within the hydrogel.

In some embodiments, compositions of the present disclosure comprise an incretin mimetic encapsulated by the hydrogel composition. The incretin mimetic can be a GLP-1 RA, such as one or more of exenatide, exenatide-LAR, lixisenatide, liraglutide, semaglutide, albiglutide, dulaglutide, efpeglenatide, ecnoglutide, efinopegdutide, cotadutide, mazdutide, BI 45690, tirzepatide, LY3493269, VK2735, CT-868, AMG133, or retatrutide. In some embodiments, the incretin mimetic is a GLP-1 RA that is an acylated peptide, such as one or more of liraglutide, semaglutide, ecnoglutide, cotadutide, mazdutide, tirzepatide, or retatrutide. Additional examples of GLP-1 RAs and incretin mimetics are provided in International Patent Application Publication Nos. WO 2005/027978 and WO 2014/005858, the disclosures of each of which are incorporated by reference herein in their entirety. Optionally, the composition can include a combination of two or more different incretin mimetics, such as two or more of any of the incretin mimetics disclosed herein.

Any of the peptides discussed herein (e.g., incretin mimetics, acylated peptides) may be referred to herein as a “therapeutic peptide.”

Optionally, the hydrogel compositions herein can include other therapeutic cargo carried by the hydrogel composition, in addition to the incretin mimetic. The other therapeutic cargo can include one or more therapeutic agents that produce a desired therapeutic effect, such as small molecule drugs, peptides, proteins, polysaccharides, nucleic acids, cells, or combinations thereof. In some embodiments, the therapeutic agent(s) act in concert with the incretin mimetic to treat the disease or condition, such as antidiabetic agents, antiobesity agents, appetite suppressants, and/or antihypertensive agents. Examples of such therapeutic agents include alpha-glucosidase inhibitors, amylin analogues (e.g., cagrilintide), biguanides, DPP4-inhibitors, glucagon antagonists, insulin and insulin analogues (e.g., insulin degludec, insulin detemir, insulin icodec, insulin glargine), meglitinides, SGLT-2 inhibitors, sulfonylureas, thiazolidinediones, and combinations thereof. Such therapeutic agents can be encapsulated in the dynamic hydrogel via physical entrapment, interactions with hydrogel components (e.g., hydrophobic interactions), or suitable combinations thereof. For instance, therapeutic agents that precipitate at physiological pH (e.g., insulin glargine) can be physically entrapped within the hydrogel. Optionally, the therapeutic agent(s) can be administered to the subject separately from the incretin mimetic via any suitable administration route (e.g., parenteral or non-parenteral administration).

### 2. Acylated Peptides

Although certain embodiments of the compositions herein are described in connection with incretin mimetics such as GLP-1 RAs, this is not intended to be limiting, and the compositions of the present technology can be used to deliver other types of therapeutic peptides, such as acylated peptides. Acylation of peptides with a lipophilic substituent (e.g., a fatty acid group) can produce improved pharmacokinetics compared to the native peptide via binding to serum albumin, while maintaining the activity of the native peptide. Other beneficial effects of acylation can include enhancing solubility, promoting cellular uptake, and/or reducing immunogenicity.

In some embodiments, a composition of the present disclosure includes at least one acylated peptide encapsulated by the hydrogel composition. The acylated peptide can include a peptide that exhibits a therapeutic effect when administered to the subject, and at least one substituent that is attached to the peptide via acylation. The peptide can be a native peptide (e.g., having 100% sequence identity to the sequence of the endogenous human peptide), or can be an analogue with one or more modifications relative to the native peptide (e.g., having less than 100% sequence identity to the sequence of the endogenous human peptide). For instance, the native peptide sequence can be modified by substitution of one or more amino acids (e.g., with a natural or non-natural amino acid), addition of one or more amino acids (e.g., a natural or non-natural amino acid), deletion of one or more amino acids, or suitable combinations thereof. The peptide can be produced in suitable host cells via recombinant DNA technology, can be produced in a cell-free system, or can be produced synthetically via solid phase synthesis.

The lipophilic substituent of the acylated peptide can have a plurality of carbon atoms, such as at least 10, 15, 20, 25, 30, 35, or 40 carbon atoms. In some embodiments, the lipophilic substituent is an acyl group of a fatty acid, such as a straight chain fatty acid or a branched fatty acid. The fatty acid can be a fatty monoacid, e.g., an aliphatic monocarboxylic acid having 4 to 38 carbon atoms, which may be saturated or unsaturated. The fatty acid can be a fatty diacid, e.g., an aliphatic dicarboxylic acid having 4 to 38 carbon atoms, which may be saturated or unsaturated. The fatty acid can be a C4 to C38 fatty acid, such as a C4 fatty acid, a C6 fatty acid, a C8 fatty acid, a C10 fatty acid, a C12 fatty acid, a C14 fatty acid, a C15 fatty acid, a C16 fatty acid, a C17 fatty acid, a C18 fatty acid, a C20 fatty acid, a C22 fatty acid, a C24 fatty acid, a C26 fatty acid, a C28 fatty acid, a C30 fatty acid, a C32 fatty acid, a C34 fatty acid, a C36 fatty acid, or a C38 fatty acid.

In some embodiments, the lipophilic substituent is an acyl group having the formula CH₃(CH₂)ₙCO—, where n is an integer from 4 to 38, or from 4 to 24, such as CH₃(CH₂)₄CO—, CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO—, CH₃(CH₂)₂₂CO—, or CH₃(CH₂)₂₄CO—.

In some embodiments, the lipophilic substituent is an acyl group having the formula HOOC(CH₂)ₙCO—, where n is an integer from 4 to 38, or from 4 to 24, such as HOOC(CH₂)₁₄CO—, HOOC(CH₂)₁₆CO—, HOOC(CH₂)₁₈CO—, HOOC(CH₂)₂₀CO—, or HOOC(CH₂)₂₂CO—.

In some embodiments, the lipophilic substituent is an acyl group of a straight-chain or branched alkane α,ω-dicarboxylic acid.

In some embodiments, the lipophilic substituent is an acyl group having the formula HOOC(CH₂)ₙCO—, where n is an integer from 4 to 38, or from 4 to 24, such as HOOC(CH₂)₁₆CO—, HOOC(CH₂)₁₈CO—, HOOC(CH₂)₂₀CO—, or HOOC(CH₂)₂₂CO.

In some embodiments, the lipophilic substituent is an acyl group having the formula CH₃(CH₂)ₙCO—NHCH(COOH)(CH₂)₂CO—, where n is an integer from 10 to 24.

In some embodiments, the lipophilic substituent is an acyl group having the formula CH₃(CH₂)ₙCO—NHCH((CH₂)₂COOH)CO—, where n is an integer from 8 to 24.

In some embodiments, the lipophilic substituent is an acyl group having the formula COOH(CH₂)ₙCO—, where n is an integer from 8 to 24.

In some embodiments, the lipophilic substituent is an acyl group having the formula —NHCH(COOH)(CH₂)₄NH—CO(CH₂)ₙCH₃, where n is an integer from 8 to 18.

In some embodiments, the lipophilic substituent interacts with a component of the dynamic hydrogel to promote encapsulation and controlled release of the acylated peptide from the dynamic hydrogels, as described further below.

In some embodiments, the lipophilic substituent is covalently attached to the peptide via an amide bond between a carboxyl group of the substituent and an amino group of the peptide. The amino group of the peptide can be the N-terminal amino group of the peptide or a side chain amino group of an amino acid residue of the peptide (e.g., an amino group of a lysine residue). The lipophilic substituent can be attached to the peptide directly, or can be attached to the peptide indirectly via a linker. For instance, the lipophilic substituent can be attached to the peptide via an amide bond between a carboxyl group of the linker and an amino group of an amino acid residue of the peptide. The linker can be any suitable linker known to those of skill in the art, such as a peptide linker (e.g., an amino acid, a γ-glutamate linker), a hydrophilic spacer (e.g., PEG, 8-amino-3,6-dioxaoctanoic acid (OEG)), a hydrophobic spacer, or a combination thereof.

In some embodiments, the acylated peptide is an acylated analogue of a proglucagon-derived peptide. Proglucagon-derived peptides are a family of peptides that are derived from differential processing of a common prohormone, proglucagon, and include glucagon, GLP-1, glucagon-like peptide-2 (GLP-2), oxyntomodulin (OXM), glicentin, glicentin-related pancreatic peptide (GRPP), intervening peptide-1 (IP-1), intervening peptide-2 (IP-2), and major proglucagon fragment (MPGF). These peptides exhibit a wide variety of physiological effects, including metabolism, energy regulation, cardioprotection, bone health, renal function, liver function, and cognition. The acylated analogue of the proglucagon peptide can include a peptide including a sequence having at least 50%, 60%, 70%, 75%, 80%, 85%, 90%, 95%, 97%, 99%, or 100% sequence identity to the native proglucagon peptide, and a lipophilic substituent attached to the peptide, as described herein.

In some embodiments, the acylated peptide is an acylated amylin analogue, such as cagrilintide. Amylin is a hormone that is secreted by pancreatic B-cells in response to nutrient ingestion. Amylin signaling plays a role in the regulation of blood glucose by delaying gastric emptying, suppressing food intake, and inhibiting meal-related glucagon secretion, and is thus complementary to the activity of the incretin hormones. The acylated amylin analogue can include a peptide including a sequence having at least 50%, 60%, 70%, 75%, 80%, 85%, 90%, 95%, 97%, 99%, or 100% sequence identity to the native amylin peptide, and a lipophilic substituent attached to the peptide, as described herein.

In some embodiments, the acylated peptide is an acylated insulin analogue, such as insulin degludec, insulin detemir, or insulin icodec. An acylated insulin analogue can be codelivered with an incretin mimetic for treatment of diabetes. The acylated insulin analogue can include a peptide including a sequence having at least 50%, 60%, 70%, 75%, 80%, 85%, 90%, 95%, 97%, 99%, or 100% sequence identity to the native insulin peptide, and a lipophilic substituent attached to the peptide, as described herein.

### 3. Vaccines

Hydrogel compositions described herein can include a vaccine (e.g., a subunit vaccine or a nucleic acid vaccine) encapsulated by the hydrogel composition. The hydrogel composition can exhibit shear-thinning behavior that allows for facile administration via injection, as well as self-healing behavior that allows for formation of a cohesive depot that delivers the vaccine over a desired treatment period. In some embodiments, upon formation of a depot in vivo, access to the vaccine is primarily or entirely limited to cells that are capable of infiltrating into the depot, such as immune cells. This approach can be used to provide selective delivery of the vaccine to a target cell population, which may be beneficial for enhancing therapeutic efficacy and/or reducing off-target effects.

In some embodiments, the vaccine is used to prevent and/or treat an infectious disease. Examples of pathogens that may be targeted by the vaccines herein include viruses, such as alphaviruses (e.g., chikungunya virus, Eastern equine encephalitis virus, Ross River virus, Venezuelan equine encephalitis virus, Western equine encephalitis virus), coronaviruses (e.g., SARS-COV-1, SARS-COV-2, MERS), filoviruses (e.g., Ebola virus, Marburg virus, Sudan virus), flaviviruses (e.g., dengue virus, yellow fever virus), hepatitis viruses (e.g., hepatitis A virus, hepatitis B virus, hepatitis C virus, hepatitis D virus, hepatitis E virus), herpes simplex virus (HSV) (e.g., HSV-1, HSV-2), human immunodeficiency virus (HIV), human papillomavirus (HPV), influenza virus (e.g., serotype A (group 1 and 2), serotype B), measles virus, mumps virus, paramyxoviruses (e.g., Hendra virus, Nipah virus), poliovirus, rabies virus, respiratory syncytial virus (RSV), rubella virus, varicella-zoster virus, variola virus, West Nile virus; and/or other microbes, such as Bacillus anthracis, Bordetella pertussis, Burkholderia cepacia, Clostridium botulinum, Clostridium tetani, Corynebacterium diphtheria, Group B Streptococcus, Haemophilus influenzae, Heliobacter pylori, Klebsiella pneumoniae, Listeria monocytogenes, Mycobacterium tuberculosis, Neisseria meningitides, Pseudomonas aeruginosa, Salmonella typhi, Shigella boydii, Shigella flexneri, Shigella dysenteriae, Shigella sonnei, Staphylococcus aureus, Streptococcus agalactiae, Streptococcus pneumonia, Streptococcus pyogenes, Vibrio cholera, Yersinia enterocolitica, Giardia duodenalis, Plasmodium falciparum, Plasmodium vivax, Toxoplasma gondii, and Trypanosoma brucei. Examples of infectious diseases that may be prevented and/or treated using the vaccines described herein include anthrax, botulism, chickenpox, chikungunya fever, cholera, COVID-19, dengue fever, diarrhea, diphtheria, Ebola, encephalitis, gastritis, gastric ulcer, giardiasis, hepatitis, herpes (e.g., oral herpes, genital herpes), Hendra virus disease, HIV/AIDS, HPV infection, influenza, listeriosis, Marburg virus disease, malaria, measles, meningitis, meningococcal disease, Middle East respiratory syndrome (MERS), mumps, Nipah virus disease, pneumococcal disease, pneumonia, polio, rabies, Ross River fever, RSV infection, rubella, severe acute respiratory syndrome (SARS), smallpox, shigellosis, shingles, sleeping sickness, streptococcal disease, tetanus, toxoplasmosis, tuberculosis, typhoid fever, West Nile fever, whooping cough, yellow fever, and yersiniosis.

In some embodiments, the nucleic acid vaccine is used to prevent and/or treat cancer. Examples of cancers that may be prevented and/or treated by the vaccines herein include biliary tract cancer, cancer (e.g., glioblastomas, medulloblastomas), breast cancer, cervical cancer, choriocarcinoma, colorectal cancer, endometrial cancer, esophageal cancer, gastric cancer, leukemia (e.g., acute lymphocytic leukemia, acute myeloid leukemia, chronic lymphocytic leukemia, chronic myeloid leukemia), liver cancer, lymphoma (e.g., Hodgkin's disease, non-Hodgkin lymphoma), lung cancer, neuroblastoma, oral cancer, ovarian cancer, pancreatic cancer, prostate cancer, renal cancer (e.g., renal cell adenocarcinoma, nephroblastoma), sarcoma (e.g., fibrosarcoma, leiomyosarcoma, liposarcoma, rhabdomyosarcoma, osteosarcoma), skin cancer (e.g., basal cell carcinoma, squamous cell carcinoma, melanoma), testicular cancer, and thyroid cancer.

In some embodiments, the vaccine is a tolerogenic vaccine that is used to treat and/or prevent an autoimmune disease or condition. Examples of autoimmune diseases or conditions that can be prevented and/or treated by the nucleic acid vaccines herein include arthritis (e.g., psoriatic arthritis, rheumatoid arthritis, ankylosing spondylitis), diabetes, inflammatory bowel disease (e.g., Crohn's disease, ulcerative colitis), multiple sclerosis (MS) (e.g., relapsing-remitting MS, secondary-progressive MS, primary-progressive MS), myasthenia gravis, pemphigus (e.g., Pemphigus vulgaris, Pemphigus foliaceus), psoriasis, system lupus erythematosus, and transplant rejection.

In some embodiments, the vaccine is a subunit vaccine. A subunit vaccine can include at least one antigen that elicits a desired immune response (e.g., a protective immune response against infection or cancer, or a tolerogenic immune response against a self-antigen). Subunit vaccines potentially have improved vaccine safety and more facile manufacturing over more traditional vaccines based on inactivated or attenuated whole pathogen vaccines. Subunit vaccines comprise at least one antigen, and optionally one or more adjuvants. For example, the FDA approved vaccine SHINGRIX, is a subunit vaccine indicated for the prevention of herpes zoster (shingles) and comprises varicella zoster virus glycoprotein E (gE) antigen and the adjuvant AS01_{B}. In some embodiments, the subunit vaccine comprises a polysaccharide vaccine, conjugate vaccine, toxoid vaccine, or a recombinant protein vaccine.

In some embodiments, the vaccine is a nucleic acid vaccine (e.g., an RNA vaccine or a DNA vaccine). The nucleic acid vaccine can include at least one nucleic acid molecule (e.g., mRNA or plasmid DNA) that encodes one or more antigens (e.g., an antigen of a pathogen associated with an infectious disease, a tumor antigen, or a self-antigen associated with an autoimmune disease). The nucleic acid molecule can encode the entire antigen (e.g., a whole protein) or a portion of the antigen (e.g., a protein fragment or peptide sequence). Optionally, the nucleic acid molecule can encode a fusion protein that includes multiple antigens as part of a single polypeptide (e.g., multiple copies of the same antigen, multiple different antigens). In some embodiments, the nucleic acid vaccine includes a single type of nucleic acid molecule that encodes for a single antigen. In other embodiments, however, the nucleic acid vaccine can include two or more different nucleic acid molecules that encode for two or more different antigens. The different antigens can be different antigens for the same disease, antigens for different diseases, or a combination thereof.

Optionally, the nucleic acid molecule can be combined with a delivery vector (e.g., a lipid or polymer vector) that encapsulates, binds to, or otherwise is combined with the nucleic acid molecule. The delivery vector can improve the delivery efficiency, and thus, the therapeutic efficacy, of the nucleic acid molecule. For instance, the nucleic acid molecule can be a mRNA and the delivery vector can be a lipid nanoparticle (LNP), such that the nucleic acid vaccine is a mRNA LNP vaccine. In other embodiments, however, different types of nucleic acid molecules and/or delivery vectors can be used. Alternatively, the nucleic acid molecule can be delivered via a viral vector, such as an adeno-associated virus (AAV) vector, an adenovirus vector (e.g., a human adenovirus vector such as huAd5, huAd46; a chimpanzee adenovirus vector such as ChAdOx1, ChAd3; a rhesus macaque adenovirus vector such as RhAd54), a flavivirus vector (e.g., a yellow fever (YF) virus vector), a herpes simplex virus (HSV) vector, a lentivirus vector, a measles virus vector, a Newcastle disease virus (NDV) vector, a poxvirus vector (e.g., a vaccinia virus vector or a variola virus (VV) vector), a retrovirus vector, or a vesicular stomatitis virus (VSV) vector.

The hydrogel compositions disclosed herein can further comprise an adjuvant to enhance the efficacy of the therapeutic treatment. The adjuvant can be a lipid-based adjuvant (e.g., a bacterial lipopolysaccharide-based adjuvant such as monophosphoryl lipid A (MPL), an emulsion-based adjuvant such as MF59), a saponin-based adjuvant (e.g., QS-21), a polynucleotide adjuvant (e.g., cytosine phosphoguanine (CpG) or immunomodulatory GpG), a metal-based adjuvant (e.g., an aluminum-based adjuvant such as amorphous aluminum hydroxyphosphate sulfate, aluminum hydroxide, aluminum phosphate, or potassium aluminum sulfate (alum)), or a combination thereof. In some embodiments, the adjuvant is a Toll-like receptor (TLR) agonist, such a TLR7/8 agonist (e.g., an imidazoquinoline such as R848, 3M052), a TLR1/2 agonist (e.g., Pam3CSK4), or a TLR2/6 agonist (e.g., Pam2CSK4). In some embodiments, the adjuvant is a NOD1 agonist, a NOD2 agonist (e.g., L18-MDP), or a NOD1/2 agonist (e.g., murabutide). The adjuvant can be a small molecule or peptide that has been functionalized with a lipid (e.g., 3M052, Pam3CSK4, Pam2CSK4, L18-MDP).

The hydrogel compositions disclosed herein can further comprise one or more immunomodulatory molecules that influence the activity of immune cells, such as antigen-presenting cells (APCs), T cells, and/or B cells. For instance, the composition can include one or more immunostimulatory molecules that recruit and/or activate immune cells, and/or one or more checkpoint inhibitors that prevent inhibition of immune cells activity. Examples of such molecules include cytokines (e.g., GM-CSF, FLT3L, IL-2, IL-10, IL-7, IL-12, IL-15, IL-21), chemokines (e.g., CCL1, CCL2, CCL3, CCL5, CCL 7, CCL8, CCL13, CCL17, CCL22, CXCL8, CXCL9, CXCL10, CXCL11), and antibodies (e.g., anti-CD40, anti-PD1).

Alternatively, in embodiments where the vaccine is a tolerogenic vaccine, the composition can include one more immunosuppressive and/or anti-inflammatory molecules that facilitate the induction of immunological tolerance. Examples of such molecules include cytokines (e.g., IL-1Rα, IL-2, IL-4, IL-10, IL-11, IL-13, TGFβ), small molecule drugs (e.g., glucocorticoids such as prednisone, dexamethasone, and hydrocortisone), and antibodies (e.g., anti-CD20, anti-TNFα). Optionally, the therapeutic agent(s) can include a combination of at least one adjuvant and at least one immunomodulatory molecule.

### C. Concentrations and Amounts

The concentrations of the first agent, second agent, and therapeutic agent may include amounts or concentrations suitable for providing the desired therapeutic effect and/or rheological properties.

The concentration of the first agent in the hydrogel composition can be varied to produce the desired hydrogel properties (e.g., stiffness, storage modulus, degradation rate). In some embodiments of the hydrogel compositions described herein, the concentration of the first agent is at least 0.1 wt %, at least 0.25 wt %, at least 0.5 wt %, at least 0.75 wt %, at least 1 wt %, at least 1.5 wt %, at least 2 wt %, at least 2.5 wt %, at least 3 wt %, at least 3.5 wt %, at least 4 wt %, at least 4.5 wt %, or at least 5 wt %; and/or the concentration of the second agent is at least 0.02 wt %, at least 0.1 wt %, at least 0.5 wt %, at least 1 wt %, at least 2 wt %, at least 3 wt %, at least 4 wt %, or at least 5 wt %.

Alternatively or in combination, the concentration of the first agent is 0.1 wt % to 5 wt %, 0.1 wt % to 4 wt %, 0.1 wt % to 3 wt %, 0.1 wt % to 2 wt %, 0.1 wt % to 1 wt %, 0.1 wt % to 0.5 wt %, 0.5 wt % to 5 wt %, 0.5 wt % to 4 wt %, 0.5 wt % to 3 wt %, 0.5 wt % to 2 wt %, 0.5 wt % to 1 wt %, 1 wt % to 5 wt %, 1 wt % to 4 wt %, 1 wt % to 3 wt %, 1 wt % to 2 wt %, 2 wt % to 5 wt %, 2 wt % to 4 wt %, 2 wt % to 3 wt %, 3 wt % to 5 wt %, 3 wt % to 4 wt %, or 4 wt % to 5 wt %; and/or the concentration of the second agent is 0.02 wt % to 5 wt %, 0.02 wt % to 4 wt %, 0.02 wt % to 3 wt %, 0.02 wt % to 2 wt %, 0.02 wt % to 1 wt %, 0.1 wt % to 5 wt %, 0.1 wt % to 4 wt %, 0.1 wt % to 3 wt %, 0.1 wt % to 2 wt %, 0.1 wt % to 1 wt %, 0.5 wt % to 5 wt %, 0.5 wt % to 4 wt %, 0.5 wt % to 3 wt %, 0.5 wt % to 2 wt %, or 0.5 wt % to 1 wt %.

Alternatively or in combination, a ratio of the concentration of the first agent to the concentration of the second agent can be greater than or equal to 1:1, 1.5:1, 2:1, 3:1, 4:1, 5:1, 10:1, 20:1, or 30:1; and/or less than or equal to 30:1, 20:1, 10:1, 5:1, 4:1, 3:1, 2:1, 1.5:1, or 1:1. The ratio of the concentration of the first agent to the concentration of the second agent can be 1:1 to 10:1, 1.5:1 to 5:1, or 2:1 to 4:1.

In some embodiments, the concentration of the first agent is 0.5 wt % to 5 wt % and the second agent is 0.01 wt % to 4 wt %, wherein the first agent is a hydrophobically modified polysaccharide (e.g., HPMC-C18); and the second agent is a surfactant (e.g., Tween 20 or Tween 80) or a cyclic polysaccharide (e.g., αCD). In some embodiments, the concentration of the first agent is 0.1 wt % to 4 wt % and the second agent is 0.01 wt % to 3 wt %, wherein the first agent is a hydrophobically modified polysaccharide (e.g., HPMC-C18); and the second agent is a surfactant (e.g., Tween 20 or Tween 80) or a cyclic polysaccharide (e.g., αCD). In some embodiments, the concentration of the first agent is 0.5 wt % to 3 wt % and the second agent is 0.2 wt % to 2 wt %, wherein the first agent is a hydrophobically modified polysaccharide (e.g., HPMC-C18); and the second agent is a surfactant (e.g., Tween 20 or Tween 80) or a cyclic polysaccharide (e.g., αCD). In some embodiments, the concentration of the first agent is 1 wt % to 3 wt % and the second agent is 0.2 wt % to 1 wt %, wherein the first agent is a hydrophobically modified polysaccharide (e.g., HPMC-C18); and the second agent is a surfactant (e.g., Tween 20 or Tween 80) or a cyclic polysaccharide (e.g., αCD).

In some embodiments, the concentration of the first agent is about 1.5 wt % and the second agent is at least about 1 wt %, wherein the first agent is a hydrophobically modified polysaccharide (e.g., HPMC-C18); and the second agent is a surfactant (e.g., Tween 20 or Tween 80) or a cyclic polysaccharide (e.g., αCD). In some embodiments, the concentration of the first agent is about 1.5 wt % and the second agent is at least about 1 wt %, wherein the first agent is HPMC-C18, and the second agent is Tween 80. In some embodiments, the concentration of the first agent is about 1.5 wt % and the second agent is about 1 wt %, wherein the first agent is HPMC-C18, and the second agent is Tween 80.

In some embodiments, the concentration of the first agent is about 1.5 wt % and the second agent is at least about 0.5 wt %, wherein the first agent is a hydrophobically modified polysaccharide (e.g., HPMC-C18); and the second agent is a surfactant (e.g., Tween 20 or Tween 80) or a cyclic polysaccharide (e.g., αCD). In some embodiments, the concentration of the first agent is about 1.5 wt % and the second agent is at least about 0.5 wt %, wherein the first agent is HPMC-C18, and the second agent is Tween 80. In some embodiments, the concentration of the first agent is about 1.5 wt % and the second agent is about 0.5 wt %, wherein the first agent is HPMC-C18, and the second agent is Tween 80.

In some embodiments, the concentration of the first agent is about 1 wt % to about 3 wt % and the second agent is about 0.25 wt % to about 1 wt %, wherein the first agent is a hydrophobically modified polysaccharide (e.g., HPMC-C18); and the second agent is a surfactant (e.g., Tween 20 or Tween 80) or a cyclic polysaccharide (e.g., αCD). In some embodiments, the concentration of the first agent is about 1 wt % to about 3 wt % and the second agent is about 0.25 wt % to about 1 wt %, wherein the first agent is HPMC-C18, and the second agent is Tween 20. In some embodiments, the concentration of the first agent is about 1 wt % and the second agent is about 0.25 wt %, wherein the first agent is HPMC-C18, and the second agent is Tween 20. In some embodiments, the concentration of the first agent is about 2 wt % and the second agent is about 0.5 wt %, wherein the first agent is HPMC-C18, and the second agent is Tween 20. In some embodiments, the concentration of the first agent is about 3 wt % and the second agent is about 0.75 wt %, wherein the first agent is HPMC-C18, and the second agent is Tween 20. In some embodiments, the concentration of the first agent is about 2 wt % and the second agent is about 1 wt %, wherein the first agent is HPMC-C18, and the second agent is Tween 20. In some of the foregoing embodiments, the hydrogel composition further comprises a vaccine (e.g., subunit vaccine or nucleic acid vaccine). In some of the foregoing embodiments, the hydrogel composition further comprises an adjuvant, wherein the adjuvant is MPL.

In some embodiments, the concentration of the first agent is about 3 wt % and the second agent is at least about 0.75 wt %, wherein the first agent is a hydrophobically modified polysaccharide (e.g., HPMC-C18); and the second agent is a surfactant (e.g., Tween 20 or Tween 80) or a cyclic polysaccharide (e.g., αCD). In some embodiments, the concentration of the first agent is about 3 wt % and the second agent is at least about 0.75 wt %, wherein the first agent is HPMC-C18, and the second agent is Tween 80. In some embodiments, the concentration of the first agent is about 3 wt % and the second agent is about 0.75 wt %, wherein the first agent is HPMC-C18, and the second agent is Tween 80.

In some embodiments, the hydrogel composition is an anti-adhesion hydrogel composition, wherein the concentration of the first agent is 1 wt % to 3 wt %, or 1 wt % to 2 wt %; and the concentration of the second agent is 0.1 wt % to 2 wt %, 0.1 wt % to 1 wt %, 0.1 wt % to 0.75 wt %, or 0.2 wt % to 0.75 wt %. In some embodiments, the concentration of the first agent is about 1.8 wt %, and the concentration of the second agent is about 0.5 wt %. In some embodiments, the first agent is HPMC-C18 and has a concentration of about 1.8 wt %, and the second agent is Tween 20 and has a concentration of about 0.5 wt %. In some embodiments, the anti-adhesion hydrogel composition does not comprise a therapeutic agent. In some embodiments, the anti-adhesion hydrogel composition further comprises a therapeutic agent.

The hydrogel composition can include any suitable amount of a therapeutic agent for providing the desired therapeutic effect. In some embodiments, wherein the hydrogel composition comprises a therapeutic agent, the amount of therapeutic agent may be provided in an amount per dose. A “dose” refers to an amount of a therapeutic agent administered to an individual at a specific time. For example, a dose may be an amount of therapeutic agent administered to an individual in a single, continuous injection of a hydrogel composition described herein.

In some embodiments, the hydrogel composition comprises a therapeutic agent in an amount per dose of 0.1 g to 200 μg, 0.1 μg to 0.25 μg, 0.25 μg to 1 μg, 1 μg to 10 μg, 10 μg to 50 μg, 50 μg to 150 μg, or 100 μg to 200 μg.

The hydrogel composition can include any suitable amount of a subunit vaccine (e.g., peptide or glycoprotein antigen) or a nucleic acid vaccine (e.g., nucleic acid molecule and delivery vector) for providing the desired therapeutic effect. For example, the hydrogel composition can include an amount per dose of at least 0.01 μg, 0.05 μg, 0.1 μg, 0.25 μg, 0.5 μg, 1 μg, 2 μg, 5 μg, 10 μg, 15 μg, 20 μg, 25 μg, 30 μg, 35 μg, 40 μg, 45 μg, 50 μg, 60 μg, 70 μg, 80 μg, 90 μg, 100 μg, 125 μg, 150 μg, 175 μg, or 200 μg of the subunit or nucleic acid vaccine. Alternatively or in combination, the hydrogel composition can include an amount per dose of no more than 200 μg, 175 μg, 150 μg, 125 μg, 100 μg, 90 μg, 80 μg, 70 μg, 60 g, 50 ug, 45 μg, 40 μg, 35 μg, 30 μg, 25 μg, 20 μg, 15 μg, 10 μg, 5 μg, 2 μg, 1 μg, 0.5 μg, 0.25 μg, 0.1 μg, or 0.05 μg of the subunit or nucleic acid vaccine. The amount per dose of the subunit or nucleic acid vaccine in the composition can be within a range from 0.01 μg to 200 μg, 0.01 μg to 150 μg, 0.01 μg to 100 g, 0.01 μg to 50 μg, 0.01 μg to 40 μg, 0.01 μg to 30 μg, 0.01 μg to 20 μg, 0.01 μg to 10 μg, 0.01 μg to 5 μg, 0.01 μg to 1 μg, 0.01 μg to 0.5 μg, 0.01 μg to 0.25 μg, 0.01 μg to 0.1 μg, 0.25 μg to 200 μg. 0.25 μg to 150 μg, 0.25 μg to 100 μg, 0.25 μg to 50 μg, 0.25 μg to 40 μg, 0.25 μg to 30 μg, 0.25 μg to 20 μg, 0.25 μg to 10 μg, 0.25 μg to 5 μg, 0.25 μg to 1 μg, 0.25 μg to 0.5 μg, 0.5 μg to 200 μg, 0.5 μg to 150 μg, 0.5 g to 100 μg, 0.5 μg to 50 μg, 0.5 μg to 40 μg, 0.5 μg to 30 μg, 0.5 μg to 20 μg, 0.5 μg to 10 μg, 0.5 μg to 5 μg, 0.5 μg to 1 μg, 1 μg to 200 μg, 1 μg to 150 μg, 1 μg to 100 μg, 1 μg to 50 μg, 1 μg to 40 μg, 1 μg to 30 μg, 1 μg to 20 μg, 1 μg to 10 μg, 1 μg to 5 μg, 5 μg to 200 μg, 5 μg to 150 g, 5 μg to 100 μg, 5 μg to 50 μg, 5 μg to 40 μg, 5 μg to 30 μg, 5 μg to 20 μg, 5 μg to 10 μg, 10 μg to 200 μg, 10 μg to 150 μg, 10 μg to 100 μg, 10 μg to 50 μg, 10 μg to 40 μg, 10 μg to 30 μg, 10 μg to 20 μg, 20 μg to 200 μg, 20 μg to 150 μg, 20 μg to 100 μg, 20 μg to 50 μg, 20 μg to 40 μg, 20 μg to 30 g, 30 μg to 200 μg, 30 μg to 150 μg, 30 μg to 100 μg, 30 μg to 50 μg, 30 μg to 40 μg, 40 μg to 200 μg, 40 μg to 150 μg, 40 μg to 100 μg, 40 μg to 50 μg, 50 μg to 200 μg, 50 g to 150 μg, 50 μg to 100 μg, 100 μg to 200 μg, 100 μg to 150 μg, or 150 μg to 200 μg. The amount per dose of the subunit or nucleic acid vaccine in the composition can be approximately 0.01 μg, 0.05 μg, 0.1 μg, 0.25 μg, 0.5 μg, 1 μg, 2 μg, 5 μg, 10 μg, 15 μg, 20 μg, 25 μg, 30 μg, 35 μg, 40 μg, 45 μg, 50 μg, 60 μg. 70 μg, 80 μg, 90 μg, 100 μg, 125 μg, 150 μg, 175 μg, or 200 μg.

In some embodiments, the therapeutic agent is an incretin mimetic or acylated peptide. In some embodiments, the amount per dose of the incretin mimetic or acylated peptide is at least 1 mg, 2 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 125 mg, 150 mg, 175 mg, 200 mg, 225 mg, 250 mg, 275 mg, 300 mg, or 400 mg. Alternatively or in combination, the amount per dose of the incretin mimetic or acylated peptide is no more than 500 mg, 400 mg, 300 mg, 275 mg, 250 mg, 225 mg, 200 mg, 175 mg, 150 mg, 125 mg, 100 mg, 90 mg, 80 mg, 70 mg, 60 mg, 50 mg, 45 mg, 40 mg, 35 mg, 30 mg, 25 mg, 20 mg, 15 mg, 10 mg, 5 mg, or 2 mg. In some embodiments, the amount per dose of the incretin mimetic or acylated peptide is 1 mg to 500 mg, 1 mg to 250 mg, 1 mg to 150 mg, 1 mg to 100 mg, 1 mg to 50 mg, 1 mg to 40 mg, 1 mg to 30 mg, 1 mg to 20 mg, 1 mg to 10 mg, 10 mg to 500 mg, 10 mg to 250 mg, 10 mg to 150 mg, 10 mg to 100 mg, 10 mg to 50 mg, 10 mg to 40 mg, 10 mg to 30 mg, 10 mg to 20 mg, 20 mg to 500 mg, 20 mg to 250 mg, 20 mg to 150 mg, 20 mg to 100 mg, 20 mg to 50 mg, 20 mg to 40 mg, 20 mg to 30 mg, 30 mg to 500 mg, 30 mg to 250 mg, 30 mg to 150 mg, 30 mg to 100 mg, 30 mg to 50 mg, 30 mg to 40 mg, 40 mg to 500 mg, 40 mg to 250 mg, 40 mg to 150 mg, 40 mg to 100 mg, 40 mg to 50 mg, 50 mg to 500 mg, 50 mg to 250 mg, 50 mg to 150 mg, 50 mg to 100 mg, 100 mg to 500 mg, 100 mg to 250 mg, 100 mg to 150 mg, 150 mg to 500 mg, 150 mg to 250 mg, or 250 mg to 500 mg. In some embodiments, the amount per dose of the incretin mimetic or acylated peptide is approximately 1 mg, 2 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 125 mg, 150 mg, 175 mg, 200 mg, 225 mg, 250 mg, 275 mg, 300 mg, 400 mg, or 500 mg.

In some embodiments, the concentration of the incretin mimetic or acylated peptide in the hydrogel composition is at least 0.5 mg/mL, 1 mg/mL, 1.5 mg/mL, 2 mg/mL, 5 mg/mL, 10 mg/mL, 20 mg/mL, 50 mg/mL, 100 mg/mL, 120 mg/mL, or 150 mg/mL. Alternatively or in combination, the concentration of the incretin mimetic or acylated peptide in the hydrogel composition is no more than 200 mg/mL, 150 mg/mL, 120 mg/mL, 100 mg/mL, 50 mg/mL, 20 mg/mL, 10 mg/mL, 5 mg/mL, 2 mg/mL, 1.5 mg/mL, 1 mg/mL, or 0.5 mg/mL. The concentration of the incretin mimetic or acylated peptide in the hydrogel composition can be 0.5 mg/mL to 200 mg/mL, 0.5 mg/mL to 150 mg/mL, 0.5 mg/mL to 120 mg/mL, 0.5 mg/mL to 100 mg/mL, 0.5 mg/mL to 50 mg/mL, 0.5 mg/mL to 20 mg/mL, 0.5 mg/mL to 10 mg/mL, 0.5 mg/mL to 5 mg/mL, 0.5 mg/mL to 2 mg/mL, 0.5 mg/mL to 1.5 mg/mL, 0.5 mg/mL to 1 mg/mL, 1 mg/mL to 200 mg/mL, 1 mg/mL to 150 mg/mL, 1 mg/mL to 120 mg/mL, 1 mg/mL to 100 mg/mL, 1 mg/mL to 50 mg/mL, 1 mg/mL to 20 mg/mL, 1 mg/mL to 10 mg/mL, 1 mg/mL to 50 mg/mL, 1 mg/mL to 2 mg/mL, 1 mg/mL to 1 mg/mL to 1.5 mg/mL, 1.5 mg/mL to 200 mg/mL, 1.5 mg/mL to 150 mg/mL, 1.5 mg/mL to 120 mg/mL, 1.5 mg/mL to 100 mg/mL, 1.5 mg/mL to 50 mg/mL, 1.5 mg/mL to 20 mg/mL, 1.5 mg/mL to 10 mg/mL, 1.5 mg/mL to 5 mg/mL, 1.5 mg/mL to 2 mg/mL, 2 mg/mL to 200 mg/mL, 2 mg/mL to 150 mg/mL, 2 mg/mL to 120 mg/mL, 2 mg/mL to 100 mg/mL, 2 mg/mL to 50 mg/mL, 2 mg/mL to 20 mg/mL, 2 mg/mL to 10 mg/mL, 2 mg/mL to 5 mg/mL, 5 mg/mL to 200 mg/mL, 5 mg/mL to 150 mg/mL, 5 mg/mL to 120 mg/mL, 5 mg/mL to 100 mg/mL, 5 mg/mL to 50 mg/mL, 5 mg/mL to 20 mg/mL, 5 mg/mL to 10 mg/mL, 10 mg/mL to 200 mg/mL, 10 mg/mL to 150 mg/mL, 10 mg/mL to 120 mg/mL, 10 mg/mL to 100 mg/mL, 10 mg/mL to 50 mg/mL, 10 mg/mL to 20 mg/mL, 20 mg/mL to 200 mg/mL, 20 mg/mL to 150 mg/mL, 20 mg/mL to 120 mg/mL, 20 mg/mL to 100 mg/mL, 20 mg/mL to 50 mg/mL, 50 mg/mL to 200 mg/mL, 50 mg/mL to 150 mg/mL, 50 mg/mL to 120 mg/mL, 50 mg/mL to 100 mg/mL, or 100 mg/mL to 200 mg/mL.

### D. Preparation

In some embodiments, the hydrogel compositions herein are prepared by simple mixing of the first agent, second agent, therapeutic agent, and any optional additives. For example, the hydrogel composition can be prepared by forming a solution of the first agent (e.g., by combining the modified cellulose polymer in an aqueous solvent such as water or a buffered solution such as phosphate-buffered saline (PBS)), forming a solution of the second agent (e.g., by combining the second agent in an aqueous solvent), and forming a solution of the therapeutic agent (e.g., by combining a therapeutic agent in an aqueous solvent). The solutions of the first agent, second agent, and therapeutic agent mixtures can then be combined, optionally with external agitation, to form the hydrogel composition including the therapeutic agent. As another example, the hydrogel composition can be prepared by forming a solution of the first agent, forming a solution of the second agent combined with a therapeutic agent, then combining the two solutions to form the hydrogel composition including the therapeutic agent. In a further example, the hydrogel composition can be prepared by forming a solution of the first agent combined with a therapeutic agent, forming a solution of the second agent, then combining the two solutions to form the hydrogel composition including the therapeutic agent. In yet another example, the hydrogel composition can be prepared by forming a solution of the first agent, forming a solution of the second agent, then combining the solutions to form the hydrogel composition without any therapeutic agent. A solution may refer to a homogeneous solution consisting of a single liquid phase, or a heterogeneous solution consisting of two or more phases (e.g., liquid and solid).

In some embodiments, the present disclosure provides methods for preparing a hydrogel composition for treating an individual as described herein. The method can include combining the components of a hydrogel composition (e.g., HPMC-C18 and Tween 20, HPMC-C18 and Tween 80, or HPMC-C18 and αCD) with a therapeutic agent (e.g., a vaccine, acylated peptide, or an incretin mimetic), thus forming a hydrogel composition containing the therapeutic agent. The combining of the hydrogel components and the nucleic acid therapeutic or incretin mimetic can be performed using simple mixing under gentle conditions, such as physiological pH (e.g., pH 7.0 to 7.4) at room temperature (e.g., 25° C.) or physiological temperature (e.g., 37° C.). Optionally, in embodiments where a nucleic acid therapeutic includes a nucleic acid molecule complexed with a delivery vector (e.g., a lipid vector or a polymer vector), the mixing can be sufficiently gentle to avoid disrupting the nucleic acid-delivery vector complex. In alternative embodiments, the method comprises combining the components of a hydrogel composition (e.g., HPMC-C18 and Tween 20, HPMC-C18 and Tween 80, or HPMC-C18 and αCD) without a therapeutic agent.

In some embodiments, the present disclosure provides kits for preparing a hydrogel composition as described herein. The kit can include a solution containing a therapeutic agent (e.g., a solution containing a subunit vaccine, nucleic acid molecules combined with a delivery vector, or a solution containing an incretin mimetic or acylated peptide) and one or more solutions containing the components of the hydrogel composition (e.g., a solution containing a first agent and a solution containing a second agent, or a single solution containing both first and second agents). The solutions can be provided in tubes, bottles, ampoules, syringes, or any other suitable storage container. In some embodiments, the solutions each independently include a suitable pharmaceutically acceptable diluent. The pharmaceutically acceptable diluent can be any diluent that does not substantially produce adverse reactions, e.g., toxic, allergic, or immunological reactions, when administered to a subject. Examples of pharmaceutically acceptable diluents include, but are not limited to, saline, Ringer's solution, dextrose solution, phosphate buffered saline, water, or a combination thereof. The pharmaceutically acceptable diluent can include an isotonicity imparting agent, such as sodium chloride, potassium chloride, or monosodium phosphate. The pharmaceutically acceptable diluent can include a buffer, such as bicarbonate, TRIS, HEPES, MOPS, CHES, CHAPS, or phosphate buffered saline. The pharmaceutically acceptable diluent can include stabilizers and/or preservatives, as appropriate. Additional examples and details of pharmaceutically acceptable diluents can be found in Martin, Remington's Pharmaceutical Sciences, 21st Ed., Mack Publ. Co., Easton, Pa. (2005), which is incorporated herein by reference in its entirety.

### II. Hydrogel Properties

The hydrogel compositions described herein may exhibit favorable physical and biological properties that contribute to their efficacy, e.g., as drug delivery platforms, anti-adhesion materials, and/or 3D printing materials. The properties of the hydrogels compositions herein can be tuned in various ways, such as by modifying the types of components used to form the hydrogel (e.g., first or second agents as previously described in Section I.A; and/or the therapeutic agent carried by the hydrogel as described below in Section I.B), the concentrations of the components, and/or the chemical functionalities of the components. Accordingly, the properties of the hydrogel compositions herein can be adapted to the particular therapeutic application, such as forming a stable and/or persistent depot when delivered in vivo, providing a desired release profile for the therapeutic cargo (e.g., short-term release versus long-term release), providing a desired release mechanism for the therapeutic cargo (e.g., diffusion-based release versus erosion-based release), compatibility with a desired route of administration (e.g., injecting, infusing, spraying, spreading), biodegradability, biocompatibility, allowing for cellular infiltration, forming a barrier to reduce/prevent tissue adhesions, and/or providing desired mechanical properties for 3D printing. Any reference herein to a property of a hydrogel composition may refer to the property of the hydrogel composition without any therapeutic cargo (e.g., a hydrogel composition composed of first and second agents without a therapeutic agent), the property of the hydrogel composition including the therapeutic cargo (e.g., a hydrogel composition including first and second agents, and a therapeutic agent), or both, unless otherwise stated or otherwise evident from the context.

In some embodiments, the hydrogel composition exhibits self-healing properties and forms a cohesive depot at the injection site. The formation of a depot can control the release of the therapeutic agent, such as an incretin mimetic, acylated peptide, or vaccine. For instance, in embodiments where the therapeutic agent is physically entrapped in the hydrogel composition and/or interacts with hydrogel components, the therapeutic agent may be released from the depot primarily or entirely via erosion of the hydrogel composition, rather than via diffusion out of the hydrogel. Accordingly, the release rate of the therapeutic agent can be controlled by tuning the degradation rate of the hydrogel composition, such as by modifying the stiffness of the hydrogel composition as described herein. In other embodiments, the therapeutic agent diffuses out of the hydrogel depot without requiring erosion of the depot.

Alternatively or in combination, the formation of a depot can be used to expose the therapeutic agent to a target cell population, with the therapeutic agent substantially remaining in the depot. The target cell population can include one or more cell types that, upon taking up the therapeutic agent, such as a vaccine, produce a desired therapeutic effect (e.g., preventing and/or treating a disease). In some embodiments, the hydrogel composition forms a mesh network that retains a therapeutic agent within the interior of the hydrogel composition. The mesh network can allow certain target cells (e.g., immune cells) to infiltrate into the hydrogel composition to take up the therapeutic agent, while substantially retaining the therapeutic agent in the depot and preventing nontarget cells from accessing the therapeutic agent. The time frame of release of the therapeutic agent from the hydrogel composition can be slower than the time frame of cell infiltration into the hydrogel composition, such that the therapeutic agent is taken up primarily or entirely by cells that infiltrate into the hydrogel composition, rather than by cells external to the hydrogel composition. This approach can enhance the therapeutic efficacy of the therapeutic agent and/or reduce side effects associated with uptake of the therapeutic agent by nontarget cells.

For instance, in embodiments where the therapeutic agent is a subunit vaccine or a nucleic acid vaccine (e.g., a mRNA LNP vaccine), immune cells can infiltrate into the hydrogel composition, while nonimmune cells at the injection site (e.g., muscle cells, skin cells, adipocytes, fibroblasts) can be mostly or entirely excluded from the hydrogel composition. The immune cells can include APCs, such as dendritic cells (e.g., cDC1s, cDC2s, iDCs), monocytes, macrophages, and B cells. Selective targeting of subunit or nucleic acid vaccines to APCs may improve vaccine efficacy, while also reducing side effects, such as injection site pain, that may be attributable to nonspecific uptake of the vaccine by nonimmune cells. In addition to promoting selective uptake of the subunit or nucleic acid vaccine by APCs, the hydrogel composition can optionally serve as a local immunological niche by recruiting other types of immune cells, such as T cells (e.g., cytotoxic T cells, helper T cells (such as TH1 cells, TH2 cells, and TH17 cells), regulatory T cells, memory T cells, γδ T cells), natural killer (NK) cells, neutrophils, basophils, eosinophils, and other myeloid and non-myeloid cells). As described herein, the hydrogel composition can optionally be further loaded with one or more therapeutic agents, such as adjuvants, cytokines, and/or chemokines, that recruit and/or activate immune cells.

The hydrogel compositions described herein are biocompatible. A biocompatible material can be a material that is, along with any metabolites or degradation products thereof, generally non-toxic to the subject, and do not cause any significant adverse effects to the subject, at concentrations resulting from the degradation of the administered materials. A biocompatible material can be a material that does not elicit a significant inflammatory or immune response when administered to a subject.

### A. Rheological Properties

The hydrogel compositions disclosed herein have measurable rheological characteristics, and can be described by their storage modulus, yield stress, viscoelasticity, shear-thinning behavior, and self-healing behavior. These characteristics are discussed below.

### 1. Storage Modulus

The storage modulus (G′) of the hydrogel composition correlates to the overall stiffness of the hydrogel, which in turn can dictate the time scale of degradation of the hydrogel (e.g., hydrogels having a higher storage modulus may be stiffer and degrade more slowly than gels having a lower storage modulus). Accordingly, in embodiments where the therapeutic agent of the hydrogel composition is released primarily or entirely via an erosion-based mechanism, the release rate of the therapeutic cargo can be tuned by adjusting the storage modulus of the hydrogel (e.g., a higher storage modulus can produce a slower degradation rate and thus a slower release rate of the therapeutic cargo, while a lower storage modulus can produce a higher degradation rate and thus a faster release rate of the therapeutic cargo). For example, the storage modulus of the hydrogel composition can be increased by increasing the concentration of the first agent (e.g., HPMC-C18). Conversely, the storage modulus of the hydrogel composition can be decreased by decreasing the concentration of the first agent (e.g., HPMC-C18).

The storage modulus can be measured, for example, using an oscillatory shear test in a parallel plate rheometer at an angular frequency of 10 rad·s⁻¹, a strain within the linear viscoelastic region of the hydrogel (e.g., 1% strain), and a temperature of 25° C. In some embodiments, the hydrogel compositions herein have a storage modulus within a range from 1 Pa to 10,000 Pa, 1 Pa to 5000 Pa, 1 Pa to 2500 Pa, 1 Pa to 1000 Pa, 1 Pa to 500 Pa, 1 Pa to 200 Pa, 1 Pa to 10 Pa, 10 Pa to 10,000 Pa, 10 Pa to 5000 Pa, 10 Pa to 2500 Pa, 10 Pa to 1000 Pa, 10 Pa to 500 Pa, 10 Pa to 200 Pa, 10 Pa to 100 Pa, 10 Pa to 50 Pa, 50 Pa to 10,000 Pa, 50 Pa to 5000 Pa, 50 Pa to 2500 Pa, 50 Pa to 1000 Pa, 50 Pa to 500 Pa, 50 Pa to 200 Pa, 50 Pa to 100 Pa, 100 Pa to 10,000 Pa, 100 Pa to 5000 Pa, 100 Pa to 2500 Pa, 100 Pa to 1000 Pa, 100 Pa to 500 Pa, 100 Pa to 200 Pa, 200 Pa to 10,000 Pa, 200 Pa to 5000 Pa, 200 Pa to 2500 Pa, 200 Pa to 1000 Pa, 200 Pa to 500 Pa, 500 Pa to 10,000 Pa, 500 Pa to 5000 Pa, 500 Pa to 2500 Pa, 500 Pa to 1000 Pa, 1000 Pa to 10,000 Pa, 1000 Pa to 5000 Pa, 1000 Pa to 2500 Pa, 2500 Pa to 10,000 Pa, 2500 Pa to 5000 Pa, or 5000 Pa to 10,000 Pa. In some embodiments, the hydrogel composition has a storage modulus within a range from 1 Pa to 500 Pa, 1 Pa to 250 Pa, 1 Pa to 100 Pa, 1 Pa to 50 Pa, 1 Pa to 25 Pa, 1 Pa to 10 Pa, 10 Pa to 500 Pa, 10 Pa to 250 Pa, 10 Pa to 100 Pa, 10 Pa to 50 Pa, 10 Pa to 25 Pa, 25 Pa to 500 Pa, 25 Pa to 250 Pa, 25 Pa to 100 Pa, 25 Pa to 50 Pa, 50 Pa to 500 Pa, 50 Pa to 250 Pa, 50 Pa to 100 Pa, 100 Pa to 500 Pa, 100 Pa to 250 Pa, or 250 Pa to 500 Pa when measured at 25° C. over an angular frequency of 0.1 rad·s⁻¹ to 100 rad·s⁻¹ within a linear viscoelastic region of the hydrogel composition. In some embodiments, the hydrogel composition has a storage modulus within a range from 10 Pa to 1000 Pa, or 50 Pa to 500 Pa when measured at 25° C. over an angular frequency of 0.1 rad·s⁻¹ to 100 rad·s⁻¹ within a linear viscoelastic region of the hydrogel composition.

The formulation of the hydrogel composition can be selected to avoid interfering with the complexation of a nucleic acid molecule with a delivery vector, as discussed in I.B.3. In some instances, stiffer hydrogel compositions may require more vigorous mixing during hydrogel formation, which may result in disruption of the nucleic acid-delivery vector complex. Weaker hydrogel compositions can therefore be advantageous for preserving the integrity of the complexes and thus may exhibit improved therapeutic activity compared to stiffer hydrogel compositions. Weaker hydrogel compositions may also promote cellular infiltration into the hydrogel, which can be beneficial for targeting the nucleic acid therapeutic to a target cell population as described further below. Additionally, although weaker hydrogel compositions may also degrade faster than stiffer hydrogel compositions, this may be acceptable or even advantageous in some instances, such as to promote cell infiltration into the hydrogel and/or when delivering relatively unstable nucleic acids such as mRNA, as discussed further below.

For example, in some embodiments, the storage modulus of the hydrogel composition encapsulating a nucleic acid vaccine comprising a nucleic acid and a delivery vector is less than or equal to 500 Pa, 250 Pa, 200 Pa, 150 Pa, 100 Pa, 75 Pa, 50 Pa, 25 Pa, or 10 Pa. The storage modulus can be within a range from 1 Pa to 500 Pa, 1 Pa to 250 Pa, 1 Pa to 100 Pa, 1 Pa to 50 Pa, 1 Pa to 25 Pa, 1 Pa to 10 Pa, 10 Pa to 500 Pa, 10 Pa to 250 Pa, 10 Pa to 100 Pa, 10 Pa to 50 Pa, 10 Pa to 25 Pa, 25 Pa to 500 Pa, 25 Pa to 250 Pa, 25 Pa to 100 Pa, 25 Pa to 50 Pa, 50 Pa to 500 Pa, 50 Pa to 250 Pa, 50 Pa to 100 Pa, 100 Pa to 500 Pa, 100 Pa to 250 Pa, or 250 Pa to 500 Pa. The storage modulus can be measured, for example, using an oscillatory shear test in a parallel plate rheometer at an angular frequency within a range from 0.1 rad·s⁻¹ to 100 rads·s⁻¹ (e.g., 10 rad·s⁻¹), a strain within the linear viscoelastic region of the hydrogel (e.g., 1% strain), and a temperature of 25° C.

### 2. Yield Stress

The yield stress (ty) of the hydrogel composition can correlate to the ability of the hydrogel to form and maintain a cohesive depot in vivo (e.g., a composition lacking a yield stress may flow rather than forming a cohesive depot). The hydrogel compositions herein can exhibit little or no flow when subjected to stresses below the yield stress. When subjected to stresses above the yield stress, the hydrogel compositions can flow, corresponding to a significant drop in observed viscosity (e.g., a decrease of at least one or two orders of magnitude). In some embodiments, the yield stress of the hydrogel compositions can be increased by increasing the concentration of the first agent (e.g., HPMC-C18) and/or by decreasing the concentration of the second agent (e.g., surfactant or cyclic polysaccharide). Conversely, the yield stress of the hydrogel composition can be decreased by decreasing the concentration of the first agent (e.g., HPMC-C18) and/or by increasing the concentration of the second agent (e.g., surfactant or cyclic polysaccharide).

The yield stress can be measured, for example, using a stress ramp or stress sweep (e.g., from 1 Pa to 100 Pa, or from 1 Pa to 1000 Pa) in a parallel plate rheometer at a temperature of 25° C. to identify the stress at which the hydrogel exhibits a drop in viscosity. In some embodiments, the hydrogel compositions herein have a yield stress within a range from 0.1 Pa to 1000 Pa, 0.1 Pa to 500 Pa, 0.1 Pa to 200 Pa, 0.1 Pa to 100 Pa, 0.1 Pa to 50 Pa, 0.1 Pa to 20 Pa, 0.1 Pa to 10 Pa, 0.1 Pa to 1 Pa, 1 Pa to 1000 Pa, 1 Pa to 500 Pa, 1 Pa to 200 Pa, 1 Pa to 100 Pa, 1 Pa to 50 Pa, 1 Pa to 10 Pa, 10 Pa to 1000 Pa, 10 Pa to 500 Pa, 10 Pa to 200 Pa, 10 Pa to 100 Pa, 10 Pa to 50 Pa, 10 Pa to 20 Pa, 20 Pa to 1000 Pa, 20 Pa to 500 Pa, 20 Pa to 200 Pa, 20 Pa to 100 Pa, 20 Pa to 50 Pa, 50 Pa to 1000 Pa, 50 Pa to 500 Pa, 50 Pa to 200 Pa, 50 Pa to 100 Pa, 100 Pa to 500 Pa, 100 Pa to 200 Pa, 200 Pa to 1000 Pa, 200 Pa to 500 Pa, or 500 Pa to 1000 Pa, when measured at 25° C., e.g., at an angular frequency of 10 rad·s⁻¹ within a linear viscoelastic region of the hydrogel composition. In some embodiments, the hydrogel composition has a yield stress within a range from 0.1 Pa to 100 Pa, 1 Pa to 50 Pa, 1 Pa to 25 Pa, 1 Pa to 10 Pa, 1 Pa to 5 Pa, 5 Pa to 100 Pa, 5 Pa to 50 Pa, 5 Pa to 25 Pa, 5 Pa to 10 Pa, 10 Pa to 100 Pa, 10 Pa to 50 Pa, 10 Pa to 25 Pa, 25 Pa to 100 Pa, 25 Pa to 50 Pa, or 50 Pa to 100 Pa when measured at 25° C., e.g., at an angular frequency of 10 rad·s⁻¹ within a linear viscoelastic region of the hydrogel composition. In some embodiments, the hydrogel composition has a yield stress within a range from 1 Pa to 500 Pa, 20 Pa to 200 Pa, or 50 Pa to 1000 Pa when measured at 25° C., e.g., at an angular frequency of 10 rad·s⁻¹ within a linear viscoelastic region of the hydrogel composition.

In some embodiments, hydrogel compositions having a lower stiffness and storage modulus can also exhibit a lower yield stress. In some embodiments, the yield stress of the hydrogel composition encapsulating a nucleic acid vaccine can be less than or equal to 100 Pa, 75 Pa, 50 Pa, 25 Pa, 10 Pa, 5 Pa, 1 Pa, 0.5 Pa, or 0.1 Pa. The yield stress can be within a range from 0.1 Pa to 100 Pa, 0.1 Pa to 50 Pa, 0.1 Pa to 25 Pa, 0.1 Pa to 10 Pa, 0.1 Pa to 5 Pa, 0.1 Pa to 1 Pa, 1 Pa to 100 Pa, 1 Pa to 50 Pa, 1 Pa to 25 Pa, 1 Pa to 10 Pa, 1 Pa to 5 Pa, 5 Pa to 100 Pa, 5 Pa to 50 Pa, 5 Pa to 25 Pa, 5 Pa to 10 Pa, 10 Pa to 100 Pa, 10 Pa to 50 Pa, 10 Pa to 25 Pa, 25 Pa to 100 Pa, 25 Pa to 50 Pa, or 50 Pa to 100 Pa. The yield stress can be measured, for example, using a stress ramp or stress sweep (e.g., from 0.1 Pa to 100 Pa, or from 0.1 Pa to 1000 Pa) in a parallel plate rheometer at a temperature of 25° C. to identify the stress at which the hydrogel exhibits a drop in viscosity.

### 3. Viscoelasticity

Viscoelasticity is a property of a material that simultaneously exhibits viscous and elastic behaviors when undergoing deformation or stress. The overall viscoelasticity is reflected by the tan delta ratio of the hydrogel composition. The tan delta of the hydrogel composition refers to the ratio of the loss modulus (G″) over the storage modulus (G′) (tan (8)=G″/G′)). This ratio can describe the overall viscoelasticity of the hydrogel where lower tan delta values correspond to more solid-like behavior, and higher tan delta values correspond to more liquid-like behavior, and can correlate to the degradation rate of the hydrogel.

The tan delta can be measured, for example, using an oscillatory shear test in a parallel plate rheometer at an angular frequency of 10 rad·s⁻¹, a strain within the linear viscoelastic region of the hydrogel (e.g., 1% strain), and a temperature of 25° C. In some embodiments, the hydrogel compositions herein have a tan delta less than or equal to 1, 0.9, 0.8, 0.7, 0.6, 0.5, 0.4, 0.3, 0.2, or 0.1. The tan delta can be within a range from 0.1 to 1, 0.1 to 0.5, 0.1 to 0.3, 0.2 to 1, 0.2 to 0.5, or 0.5 to 1.

In some embodiments, the hydrogel compositions herein exhibit viscoelastic behavior, wherein the storage modulus (G′) of the hydrogel is equal to the loss modulus (G″) at a crossover point, for example, a crossover frequency as observed in an oscillatory frequency sweep measurement in a range from 0.1 rad·s⁻¹ to 100 rad·s⁻¹ on an oscillatory rheometer performed in the linear viscoelastic region at a temperature of 25° C. In some embodiments, the crossover point of a hydrogel composition including both the first agent and the second agent is greater than the crossover point of a hydrogel composition including the first agent only (e.g., the crossover frequency of a composition with the first and second agents is shifted toward higher frequencies in an oscillatory frequency sweep measurement compared to the crossover point of a composition with the first agent but not the second agent). In some embodiments, the crossover frequency of a hydrogel composition including both the first agent and the second agent is greater than or equal to 0.001 rad·s⁻¹, 0.01 rad·s⁻¹, or 0.1 rad·s⁻¹, e.g., as measured in an oscillatory frequency sweep measurement in a parallel plate rheometer in the linear viscoelastic region at a temperature of 25° C. The crossover frequency is inversely correlated to the relaxation time of the composition, which is indicative of the dynamic or static nature of the composition (e.g., longer relaxation times correspond to more static materials, while shorter relaxation times correspond to more dynamic materials).

### 4. Shear-Thinning Behavior

The hydrogel compositions disclosed herein exhibit shear-thinning behavior, wherein the viscosity of the hydrogel composition decreases with increasing shear rate and/or shear stress. Shear-thinning behavior can be advantageous, for example, to allow the hydrogel composition to be administered via injection. In some embodiments, the viscosity of the hydrogel composition decreases with increasing shear rate at a shear rate within a range from 0.1 s⁻¹ to 1000 s⁻¹, for example, as observed on an oscillatory rheometer (e.g., a parallel plate rheometer) at 25° C.

The viscosity can be measured, for example, using steady shear measurements in a parallel plate rheometer at a temperature of 25° C. In some embodiments, the hydrogel compositions herein have a viscosity within a range from 10 mPa-s to 2000 mPa-s, 10 mPa-s to 1000 mPa-s, 10 mPa-s to 500 mPa-s, 10 mPa-s to 200 mPa-s, 10 mPa-s to 100 mPa-s, 10 mPa-s to 50 mPa-s, 50 mPa-s to 2000 mPa-s, 50 mPa-s to 1000 mPa-s, 50 mPa-s to 500 mPa-s, 50 mPa-s to 200 mPa-s, 50 mPa-s to 100 mPa-s, 100 mPa-s to 2000 mPa-s, 100 mPa-s to 1000 mPa-s, 100 mPa-s to 500 mPa-s, 100 mPa-s to 200 mPa-s, 200 mPa-s to 2000 mPa-s, 200 mPa-s to 1000 mPa-s, 200 mPa-s to 500 mPa-s, 500 mPa-s to 2000 mPa-s, 500 mPa-s to 1000 mPa-s, or 1000 mPa-s to 2000 mPa-s at a shear rate of 1000 s⁻¹. In some embodiments, the viscosity can be less than 10,000 mPa-s, 1000 mPa-s, or 100 mPa-s at a shear rate of 1000 s⁻¹.

### 5. Self-Healing Behavior

In some embodiments, the hydrogel compositions herein exhibit self-healing behavior. Self-healing may refer to a process in which a material that exhibits reduced resistance to flow when subjected to an external stress regains some or all of its rigidity and/or strength after the external stress is removed. Self-healing behavior can be advantageous, for example, to allow the hydrogel composition to form a cohesive depot after administration via injection and/or to limit burst release of an encapsulated therapeutic agent. In some embodiments, the hydrogel compositions described herein stop flowing and recover their mechanical properties in no more than 5 seconds, 10 seconds, 30 seconds, 1 minute, 2 minutes, 3 minutes, 5 minutes, or 10 minutes after the external stress is removed. In some embodiments, the hydrogel compositions described herein stop flowing and recover their mechanical properties in no more than 5 seconds or 10 seconds after the external stress is removed. Optionally, the modulus and/or viscosity of the hydrogel composition can recover to at least 90% of the initial value before application of the external stress within 5 minutes in a step-strain measurement (conducted with strains of 0.5% and 500%) or step-shear measurement (conducted with shear rates of 0.1 s⁻¹ and 100 s⁻¹), respectively, on an oscillatory rheometer.

### B. Drug Release Properties

In some embodiments, the hydrogel compositions described herein are biodegradable. A biodegradable material can be a material that will degrade or erode under physiological conditions to smaller units or chemical species that are capable of being metabolized, eliminated, or excreted by the subject. For example, upon in vivo administration to a subject, the hydrogel composition can dissolve as the non-covalent bonds dissociate. The degradation rate of the hydrogel composition can be varied as desired, e.g., depending on the desired release profile for the therapeutic cargo.

In drug delivery applications, the therapeutic agent over time may diffuse out from or otherwise be released from the hydrogel composition into the surrounding tissues. The hydrogel compositions described herein may increase the residence time of the therapeutic agent in the injection site compared to formulations without the first and second agents or other formulations. For instance, injection of a hydrogel composition comprising an incretin or an incretin mimic may be gradually released from the hydrogel composition over a prolonged period of time into the areas/tissues surrounding the injection site of an individual with Type 2 diabetes. In some embodiments, the hydrogel composition delivers a therapeutic agent to the individual in need thereof over a period of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 28 days, at least 35 days, at least 40 days, at least 50 days, at least 60 days, at least 70 days, at least 80 days, at least 90 days, at least 100 days, at least 110 days, at least 120 days, at least 150 days, at least 180 days, at least 6 months, at least 7 months, at least 8 months, at least 9 months, at least 10 months, at least 11 months, at least 12 months, or at least 1 year. In some embodiments the hydrogel composition delivers a therapeutic agent to the individual in need thereof over a period of about 2 weeks, about 4 weeks, about 1 month, about 2 months, about 3 months, about 4 months, about 5 months, about 6 months, about 9 months, or about 12 months.

In some embodiments, the treatment period, wherein the individual is exposed to the therapeutic agent, can be the same or similar as the time period in which the hydrogel composition remains as a cohesive depot at the injection site. The treatment period can be shortened by increasing the degradation rate of the hydrogel composition. In some embodiments, the hydrogel composition has a relatively low stiffness, storage modulus, and/or yield stress to produce a faster degradation rate and, thus, a shorter treatment period. For instance, such hydrogel compositions can benefit delivery of nucleic acid vaccines, wherein weaker hydrogels can provide enhanced cellular infiltration and/or avoid disruption of nucleic acid-delivery vector complexes, as described herein.

The hydrogel compositions herein can be configured to deliver a therapeutically effective amount of the therapeutic agent over a desired treatment period, which can be a duration that is effective to prevent the individual from contracting the disease or condition, and/or to ameliorate or prevent a symptom of a disease or condition in the individual. For example, the treatment period can be at least 1 day, 2 days, 3 days, 4 days, 5 days, 6 days, 7 days, 8 days, 9 days, 10 days, 11 days, 12 days, 13 days, 14 days, 28 days, 35 days, 40 days, 50 days, 60 days, 70 days, 80 days, 90 days, 100 days, 110 days, 120 days, 150 days, 180 days, 6 months, 7 months, 8 months, 9 months, 10 months, 11 months, 12 months, or 1 year. The treatment period can be approximately 2 weeks, 4 weeks, 1 month, 2 months, 3 months, 4 months, 5 months, 6 months, 9 months, or 12 months.

In embodiments where the nucleic acid therapeutic includes a relatively unstable nucleic acid molecule, the hydrogel composition can deliver the nucleic acid therapeutic for a shorter treatment period. For instance, nucleic acid molecules such as mRNA may spontaneously degrade even when encapsulated within the hydrogel composition (e.g., due to hydrolysis from the presence of water molecules in the hydrogel). Accordingly, to ensure that the nucleic acid molecule is delivered before it degrades, the hydrogel composition can be configured to deliver the nucleic acid therapeutic for a shorter treatment period, such as a treatment period less than or equal to 14 days, 12 days, 10 days, 8 days, 7 days, 6 days, 5 days, 4 days, 3 days, 2 days, or 1 day. The treatment period can be within a range from 1 day to 14 days, 1 day to 12 days, 1 day to 10 days, 1 day to 7 days, 4 days to 12 days, 5 days to 10 days, 7 days to 14 days, 7 days to 10 days, 8 days to 12 days, or 8 days to 10 days.

### C. Persistence of the Hydrogel Composition

In some embodiments, following in vivo administration, the hydrogel compositions are designed to persist at the administration site (e.g., remain as a cohesive depot). Persistence of the hydrogel composition may benefit administration of subunit or nucleic acid vaccines, wherein a long residence time of the therapeutic agent is desired. Persistence of the hydrogel composition may also benefit drug delivery applications where a prolonged and/or slow delivery of a therapeutic agent, such as an incretin mimetic or acylated peptide, is desired.

In some embodiments, the hydrogel compositions described herein persist at the administration site for at least 1 day, 2 days, 3 days, 4 days, 5 days, 6 days, 7 days, 8 days, 9 days, 10 days, 11 days, 12 days, 13 days, 14 days, 21 days, 28 days, 1 month, 2 months, 3 months, 4 months, 5 months, 6 months, 9 months, or 12 months. Alternatively or in combination, the hydrogel compositions herein can persist at the administration site for no more than 12 months, 9 months, 6 months, 5 months, 4 months, 3 months, 2 months, 1 month, 28 days, 21 days, 14 days, 13 days, 12 days, 11 days, 10 days, 9 days, 8 days, 7 days, 6 days, 5 days, 4 days, 3 days, 2 days, or 1 day.

In some embodiments, following in vivo administration, the hydrogel compositions are designed to persist at the administration site (e.g., remain as a cohesive depot) for at least 1 day, 2 days, 3 days, 4 days, 5 days, 6 days, 7 days, 8 days, 9 days, 10 days, 11 days, 12 days, 13 days, 14 days, 21 days, or 28 days. Alternatively or in combination, the hydrogel compositions herein can persist at the administration site for no more than 28 days, 21 days, 14 days, 13 days, 12 days, 11 days, 10 days, 9 days, 8 days, 7 days, 6 days, 5 days, 4 days, 3 days, 2 days, or 1 day.

### III. Treatment of Individuals

The present disclosure provides methods for preventing and/or treating a disease or condition in an individual in need thereof by administering a hydrogel composition as described herein. The hydrogel composition can prevent and/or treat a disease or condition by producing a desired therapeutic effect in the individual, such as alleviation of symptoms, a reduction in the severity of the disease or condition, inhibiting an underlying cause of the disease or condition, steadying the disease or condition in a non-advanced state, delaying the progress of a disease or condition, improving or alleviating the disease or condition, and/or preventing the individual from contracting a disease or condition. In some embodiments, the disease or condition is an infectious disease, cancer, an autoimmune disease or condition, diabetes, a diabetes-related condition, or tissue adhesion.

An “individual” or “subject” is a mammal, e.g., a human, mouse, rat, guinea pig, dog, cat (e.g., domestic or zoo cats), horse, cow, pig, or non-human primate, such as a monkey, chimpanzee, baboon or rhesus macaque. In some embodiments, the individual is a human.

### A. Diseases and Conditions

### 1. Infectious Diseases

The present disclosure provides compositions and methods for preventing and/or treating an infectious disease. In some embodiments, the infectious disease is selected from the group consisting of anthrax, botulism, chickenpox, chikungunya fever, cholera, COVID-19, dengue fever, diarrhea, diphtheria, Ebola, encephalitis, gastritis, gastric ulcer, giardiasis, hepatitis, herpes (e.g., oral herpes, genital herpes), Hendra virus disease, HIV/AIDS, HPV infection, influenza, listeriosis, Marburg virus disease, malaria, measles, meningitis, meningococcal disease, MERS, mumps, Nipah virus disease, pneumococcal disease, pneumonia, polio, rabies, Ross River fever, RSV infection, rubella, SARS, smallpox, shigellosis, shingles, sleeping sickness, streptococcal disease, tetanus, toxoplasmosis, tuberculosis, typhoid fever, West Nile fever, whooping cough, yellow fever, and yersiniosis.

In some embodiments, the present disclosure provides a method of preventing and/or treating an infectious disease comprising administering a hydrogel composition, as described herein, to an individual in need thereof. The hydrogel composition can be administered to the subject via injection (e.g., a subcutaneous or intramuscular injection). The hydrogel composition can include a therapeutically effective amount of a therapeutic agent for preventing and/or treating the infectious disease. For instance, in embodiments where the hydrogel composition includes a subunit or nucleic acid vaccine (e.g., a mRNA LNP vaccine), the therapeutically effective amount can be an amount sufficient to elicit an immune response in the subject that prevents and/or treats the infectious disease. Optionally, the composition can include at least one additional therapeutic agent, such as an adjuvant, cytokine, and/or chemokine. The components of the hydrogel composition can be selected to provide injectability, formation of a cohesive depot in vivo, controlled release of the therapeutic agent(s), and/or selective infiltration of the depot by a target cell population (e.g., immune cells such as APCs). For example, the treatment period can be less than or equal to 14 days, 12 days, 10 days, 8 days, 7 days, 6 days, 5 days, 4 days, 3 days, 2 days, or 1 day.

### 2. Cancer

The present disclosure provides hydrogel compositions and methods for preventing and/or treating cancer. In some embodiments, the cancer is selected from the group consisting of biliary tract cancer, bladder cancer, brain cancer (e.g., glioblastomas, medulloblastomas), breast cancer, cervical cancer, choriocarcinoma, colorectal cancer, endometrial cancer, esophageal cancer, gastric cancer, leukemia (e.g., acute lymphocytic leukemia, acute myeloid leukemia, chronic lymphocytic leukemia, chronic myeloid leukemia), liver cancer, lymphoma (e.g., Hodgkin's disease, non-Hodgkin lymphoma), lung cancer, neuroblastoma, oral cancer, ovarian cancer, pancreatic cancer, prostate cancer, renal cancer (e.g., renal cell adenocarcinoma, nephroblastoma), sarcoma (e.g., fibrosarcoma, leiomyosarcoma, liposarcoma, rhabdomyosarcoma, osteosarcoma), skin cancer (e.g., basal cell carcinoma, squamous cell carcinoma, melanoma), testicular cancer, and thyroid cancer.

In some embodiments, the present disclosure provides a method of preventing and/or treating cancer comprising administering a hydrogel composition, as described herein, to an individual in need thereof. The hydrogel composition can be administered to the subject via injection (e.g., a subcutaneous or intramuscular injection). The hydrogel composition can include a therapeutically effective amount of a therapeutic agent for preventing and/or treating the cancer. For instance, in embodiments where the hydrogel composition includes a subunit or nucleic acid vaccine (e.g., a mRNA LNP vaccine), the therapeutically effective amount can be an amount sufficient to elicit an immune response in the subject that prevents and/or treats the cancer. Optionally, the hydrogel composition can include at least one additional therapeutic agent and/or a second nucleic acid therapeutic encoding the at least one additional therapeutic agent, such as an adjuvant, a cytokine, a chemokine, and/or a checkpoint inhibitor. The components of the hydrogel composition can be selected to provide injectability, formation of a cohesive depot in vivo, controlled release of the therapeutic agent(s), and/or selective infiltration of the depot by a target cell population (e.g., immune cells such as APCs). For example, the treatment period can be less than or equal to 14 days, 12 days, 10 days, 8 days, 7 days, 6 days, 5 days, 4 days, 3 days, 2 days, or 1 day.

### 3. Autoimmune Diseases and Related Conditions

The present disclosure provides compositions and methods for preventing and/or treating an autoimmune disease or condition. In some embodiments, the autoimmune disease or condition is selected from the group consisting of arthritis (e.g., psoriatic arthritis, rheumatoid arthritis, ankylosing spondylitis), inflammatory bowel disease (e.g., Crohn's disease, ulcerative colitis), multiple sclerosis (MS) (e.g., relapsing-remitting MS, secondary-progressive MS, primary-progressive MS), myasthenia gravis, pemphigus (e.g., Pemphigus vulgaris, Pemphigus foliaceus), psoriasis, system lupus erythematosus, and transplant rejection.

In some embodiments, the present disclosure provides a method of preventing and/or treating an autoimmune disease or condition comprising administering a hydrogel composition, as described herein, to an individual in need thereof. The hydrogel composition can be administered to the subject via injection (e.g., a subcutaneous or intramuscular injection). The hydrogel composition can include a therapeutically effective amount of a therapeutic agent for preventing and/or treating the autoimmune disease. For instance, in embodiments where the hydrogel composition includes a tolerogenic subunit or nucleic acid vaccine (e.g., a mRNA LNP vaccine), the therapeutically effective amount can be an amount sufficient to elicit immunological tolerance in the subject. Optionally, the hydrogel composition can include at least one additional therapeutic agent and/or a nucleic acid molecule encoding the at least one additional therapeutic agent, such as an immunosuppressive molecule. The components of the hydrogel composition can be selected to provide injectability, formation of a cohesive depot in vivo, controlled release of the therapeutic agent(s), and/or selective infiltration of the depot by a target cell population (e.g., immune cells such as APCs). For example, the treatment period can be less than or equal to 14 days, 12 days, 10 days, 8 days, 7 days, 6 days, 5 days, 4 days, 3 days, 2 days, or 1 day.

### 4. Diabetes and Related Conditions

The present disclosure provides compositions and methods for preventing and/or treating diabetes and/or related conditions. Examples of diabetes and/or related conditions that may be treated using the compositions described herein include prediabetes, type 1 diabetes, type 2 diabetes, hyperglycemia, impaired glucose tolerance, obesity or excessive body weight, eating disorders (e.g., bulimia nervosa, binge eating disorder), cardiovascular disease (e.g., hypertension, atherosclerosis, myocardial infarction, coronary heart diseases), liver disease (e.g., non-alcoholic fatty liver disease), neurological and/or neurodegenerative diseases (e.g., Alzheimer's disease, Parkinson's disease, amyotrophic lateral sclerosis, peripheral neuropathy, ischemia, stroke, multiple sclerosis), inflammatory diseases (e.g., asthma, psoriasis, inflammatory bowel disease), renal diseases, bone diseases (e.g., bone fragility, osteoporosis), hormonal diseases (e.g., polycystic ovary syndrome), and gastrointestinal diseases (e.g., short bowel syndrome).

In some embodiments, the present disclosure provides a method of preventing and/or treating diabetes and/or related conditions comprising administering a hydrogel composition, as described herein, to an individual in need thereof. The hydrogel composition can be administered to the subject via injection (e.g., a subcutaneous or intramuscular injection). In some embodiments, the hydrogel composition comprises a therapeutically effective amount of a therapeutic agent (e.g., an incretin mimetic such as a GLP-1 RA) for treating the diabetes and/or diabetes-related condition. The therapeutic agent may be encapsulated in the hydrogel composition and released in an amount and rate that results in the sustained reduction and/or regulation of the subject's blood glucose levels over the treatment period. The rate of release of a therapeutically effective amount of the therapeutic agent can be within a range from 0.1 mg/week to 25 mg/week, 0.1 mg/week to 5 mg/week, 0.5 mg/week to 20 mg/week, 0.5 mg/week to 2 mg/week, 0.75 mg/week to 1.5 mg/week, 1 mg/week to 2 mg/week, 5 mg/week to 10 mg/week, 10 mg/week to 20 mg/week, or 10 mg/week to 15 mg/week. The rate of release of a therapeutically effective amount of the therapeutic agent may vary depending on the type of therapeutic agent, e.g., the therapeutically effectively amount may be approximately 1 mg/week for semaglutide; approximately 12.6 mg/week (1.8 mg/day) for liraglutide; approximately 2.5 mg/week, 5 mg/week, 7.5 mg/week, 10 mg/week, 12.5 mg/week, or 15 mg/week for tirzepatide; and approximately 1 mg/week, 4 mg/week, 8 mg/week, or 12 mg/week for retatrutide. The components of the hydrogel composition are selected to provide injectability, formation of a cohesive depot in vivo, and controlled release of the therapeutic agent over a sufficiently long period for treatment of the diabetes and/or diabetes-related condition. For example, the treatment period can be at least 1 month, 2 months, 3 months, 4 months, 5 months, 6 months, 9 months, or 12 months. For example, the hydrogel composition can comprise a first agent hydrophobically modified cellulosic derivative (e.g., HPMC-C18) and a second agent such as a surfactant (e.g., Tween 20, Tween 80) or a (e.g., cyclic polysaccharide (e.g., αCD). Optionally, the hydrogel composition can include at least one dispersing agent that inhibits aggregation of the therapeutic agent, such as a surfactant (e.g., Tween 20) and/or a tonicity agent (e.g., propylene glycol).

### 5. Prevention/Reduction of Tissue Adhesion

The present disclosure provides compositions and methods for preventing and/or reducing tissue adhesion in an individual in need thereof comprising administering to an individual a hydrogel composition described herein. In some embodiments, the composition is a dynamic, injectable, and spreadable hydrogel composition that can be applied to tissue as part of a surgical procedure. In some embodiments, the hydrogel composition provides a lubricious, physical barrier to maintain movement in a tissue (e.g., tendon, ligament, joint, muscle) while preventing adhesion formation.

In some embodiments, the hydrogel composition is formulated as an adhesion barrier (e.g., a barrier to scar tissue formation) to maintain separation between tissues and/or organs, thus preventing or reducing tissue adhesion. In such a capacity, the hydrogel composition comprises one or more of the following properties: (i) tunable shear-thinning and rapid self-healing to enable spraying or spreading on the tissue of interest, (ii) tissue adherence to ensure local retention over clinically relevant timeframes, (iii) high degree of biocompatibility, and/or (iv) viscoelasticity to allow organs and tissues to freely move relative to one another to effectively prevent adhesions. Instead of providing a solid barrier between tissues and organs, the hydrogel compositions disclosed herein advantageously create a shear-thinning and viscoelastic barrier between tissues, similar to the body's natural state. These compositions exhibit highly tunable viscoelastic mechanical properties, shear-thinning, and rapid self-healing, which altogether allows them to be deployed through simple spraying or spreading using standard equipment, or by catheter delivery or direct injection. Further, the hydrogel compositions do not appreciably swell like most covalently cross-linked hydrogels, because they typically dissolve as their dynamic crosslinks dissociate.

The anti-adhesion hydrogel compositions described herein can be used in a variety of surgeries to line tissue and prevent adhesions, such as in abdominal surgeries, orthopedic surgeries, gynecologic surgeries, cardiac surgeries, or thoracic surgeries. In some embodiments the method comprises forming an incision in tissue; applying a hydrogel composition, as described herein, to tissue through the incision; and closing the incision with the hydrogel composition therein, wherein the hydrogel composition prevents a formation of adhesions between tissues and/or organs. In some embodiments, the incision is part of a surgical procedure. In some embodiments, the tissue comprises abdominal, orthopedic, thoracic, cardiac, or gynecologic tissue. In some embodiments, the hydrogel composition is administered to the individual by spraying, spreading, or injecting.

In some embodiments, the method comprises forming an incision in tissue; applying a hydrogel composition described herein to tissue through the incision; and closing the incision with the hydrogel composition therein. In some embodiments, the incision is part of a cardiac surgical procedure. In some embodiments the tissue comprises cardiac tissue. In some embodiments, the incision is part of a surgical procedure to repair a tendon, muscle, or ligament. In some embodiments the tissue comprises tendon, muscle, ligament, or related tissues. In some embodiments, the hydrogel composition is injected onto the tissue. In some embodiments, the hydrogel composition comprises a first agent, wherein the first agent is a hydrophobically modified polysaccharide (e.g., HPMC-C18); and a second agent, wherein the second agent is a surfactant (e.g., Tween 20 or Tween 80) or a cyclic polysaccharide (e.g., cyclodextrin). In some embodiments, the concentration of the first agent is 1 wt % to 3 wt %, or 1 wt % to 2 wt %; and the concentration of the second agent is 0.1 wt % to 2 wt %, 0.1 wt % to 1 wt %, 0.1 wt % to 0.75 wt %, or 0.2 wt % to 0.75 wt %. In some embodiments, the concentration of the first agent is about 1.8 wt %, and the concentration of the second agent is about 0.5 wt %. In some embodiments, the first agent is HPMC-C18 and has a concentration of about 1.8 wt %, and the second agent is Tween 20 and has a concentration of about 0.5 wt %. In some embodiments, the hydrogel composition does not comprise a therapeutic agent. In some embodiments, the hydrogel composition further comprises a therapeutic agent.

In some embodiments, the anti-adhesion hydrogel composition described herein can be applied to a tissue in the body (e.g., after surgical incision) and can remain on the tissue (e.g., within or proximate to the incision site) during a time frame that adhesions typically form and/or grow. Remaining on the tissue for at least part of the time that adhesions would otherwise form or grow can advantageously prevent tissue adhesion. In some embodiments, the hydrogel composition remains on the tissue for at least 7 days, at least 10 days, at least 14 days, or at least 21 days. Further, in some embodiments, the anti-adhesion hydrogel composition described herein can dissipate from the tissue after the time frame. In some embodiments, the anti-adhesion hydrogel composition described herein can be gone or fully dissipated from the tissue in less than 120 days, less than 100 days, less than 50 days, less than 30 days, or less than 20 days from the time of the initial administration to the tissue.

The hydrogel composition can be formulated such that applying shear force allows the hydrogel composition to have a viscous flow so as to conform to and cover the tissue. The hydrogel composition can adhere to the tissue without delaminating after conforming to and covering the tissue. In some embodiments, the hydrogel composition stops flowing and recovers its mechanical properties within 5 seconds after applying shear. In some embodiments, the storage modulus of the hydrogel composition is within a range from 10 Pa to 1000 Pa and/or the yield stress is within a range from 10 Pa to 1000 Pa, e.g., when measured at 10 rad·s⁻¹ in an oscillatory shear test in a parallel plate rheometer at a strain within the linear viscoelastic region and at a temperature of 25° C. In some embodiments, the hydrogel composition maintains a linear viscoelasticity at strains up to at least 0.5%. In some embodiments, a tan delta of the hydrogel composition is less than 1.

### B. Methods of Administration

The hydrogel compositions herein can be administered to the individual in need thereof via any suitable route. For example, in some embodiments, the hydrogel composition is administered to the individual via injection (e.g., subcutaneous injection, intramuscular injection, intraarticular injection, intralesional injection, or epidural injection). The shear-thinning properties of the hydrogel composition can allow for facile delivery of the hydrogel and the encapsulated therapeutic cargo via injection.

Injection of the hydrogel composition can be performed using any suitable tubular device having a lumen configured for delivery of a hydrogel, such as needles (e.g., hypodermic needles, surgical needles, infusion needles), injector pens, catheters, trocars, cannulas, tubing, etc.

The hydrogel composition can be injected into any suitable site in the individual's body, such as an arm, thigh, abdomen, or buttock.

The composition can be formulated to have a volume that is sufficiently small for injection, such as a volume less than or equal to 2 mL, 1.75 mL, 1.5 mL, 1.25 mL, 1 mL, 0.75 mL, 0.5 mL, or 0.25 mL.

In some embodiments, the composition is administered as a single injection at a single injection site, while in other embodiments, the composition can be administered as multiple injections at the same or different injection sites.

### C. Frequency of Administration

In some embodiments where the hydrogel composition comprises a therapeutic agent, the hydrogel composition may prolong the duration of therapeutic agent exposure to the individual. The result is a sustained and/or prolonged therapeutic effect for the treated individual. The longer lasting effect may allow a therapeutic agent to injected at a reduced frequency of time, for instance, periodically instead of daily, or every month instead of weekly, as compared to injecting the therapeutic agent in the absence of the hydrogel composition described herein.

The hydrogel composition can be administered to an individual in need thereof according to any suitable timing. For instance, the hydrogel composition can be administered on a periodic basis, such as once per week, once per 2 weeks, once per 4 weeks, once per month, once per 2 months, once per 3 months, once per 4 months, once per 5 months, once per 6 months, once per 9 months, once per year, once per 2 years, once per 5 years, or once per 10 years.

In embodiments where the therapeutic agent is a vaccine, the vaccine can be administered according to a vaccination protocol. The vaccination protocol may include a priming dose followed by one or more boost doses (e.g., one, two, three, or more boost doses).

The interval between the priming dose and first boost dose can be within a range from 1 week to 12 weeks, 1 week to 10 weeks, 1 week to 8 weeks, 2 weeks to 10 weeks, 2 weeks to 8 weeks, 2 weeks to 4 weeks, 3 weeks to 8 weeks, 4 weeks to 8 weeks, or 1 year to 2 years.

The interval between subsequent boost doses can be within a range from 1 month to 12 months, 1 month to 6 months, 1 month to 3 months, 2 months to 6 months, 2 months to 3 months, 2 months to 4 months, 3 months to 6 months, 3 months to 4 months, 4 months to 6 months, 1 year to 2 years, 2 years to 5 years, or 5 years to 10 years.

The dosage of the vaccine can be the same for the priming dose and the boost dose(s), or can be different (e.g., the priming dose can be a higher dosage than the boost dose(s)). In embodiments where multiple boost doses are used, each boost dose can have the same dosage, or some or all of the boost doses can have different dosages. Optionally, the vaccination schedule can include the priming dose only, without any boost doses.

In some embodiments, the hydrogel composition delivers a therapeutic agent to the individual in need thereof over a treatment period. During the treatment period, the hydrogel composition can deliver the therapeutic agent at a rate of approximately 0.1 mg/week, 0.25 mg/week, 0.5 mg/week, 1 mg/week, 1.5 mg/week, 2 mg/week, 2.5 mg/week, 3 mg/week, 4 mg/week, 5 mg/week, 6 mg/week, 7 mg/week, 8 mg/week, 9 mg/week, 10 mg/week, 11 mg/week, 12 mg/week, 15 mg/week, 20 mg/week, or 25 mg/week.

The delivery rate can be within a range from 0.1 mg/week to 25 mg/week, 0.25 mg/week to 25 mg/week, 0.5 mg/week to 20 mg/week, 0.5 mg/week to 2 mg/week, 0.5 mg/week to 1.5 mg/week, 1 mg/week to 2 mg/week, 2 mg/week to 5 mg/week, 5 mg/week to 15 mg/week, 5 mg/week to 10 mg/week, 10 mg/week to 20 mg/week, 10 mg/week to 15 mg/week, 12 mg/week to 25 mg/week, 12 mg/week to 15 mg/week, 15 mg/week to 25 mg/week, 15 mg/week to 20 mg/week, or 20 mg/week to 25 mg/week. Alternatively or in combination, the hydrogel composition can deliver the therapeutic agent at a rate of approximately 0.5 wt %/day, 0.6 wt %/day, 0.7 wt %/day, 0.8 wt %/day, 0.9 wt %/day, 1 wt %/day, 1.25 wt %/day, 1.5 wt %/day, 1.75 wt %/day, 2 wt %/day, 2.5 wt %/day, 3 wt %/day, 4 wt %/day, or 5 wt %/day, wherein the wt % of the therapeutic agent is measured relative to the total amount of the therapeutic agent initially present in the hydrogel composition. In some embodiments, the therapeutic agent is an incretin mimetic or an acylated peptide and is delivered at a rate over a treatment period as described above.

### D. Blood Serum Concentrations of Incretin Mimetics or Acylated Peptide.

Release of the therapeutic agent from the hydrogel composition may result in measurable concentrations of the therapeutic agent in blood serum of a treated individual. For instance, after administration of a hydrogel composition to an individual, wherein the hydrogel comprises a therapeutic amount of an incretin mimetic or acylated peptide, the blood serum of the individual may be sampled and the concentration of the therapeutic agent measured.

In some embodiments, when administered to a subject in vivo, the hydrogel composition produces a steady state concentration (C_{steady-state}) and/or mean concentration at steady state of the incretin mimetic or acylated peptide in serum of approximately 10 ng/ml, 25 ng/ml, 50 ng/mL, 75 ng/mL, 100 ng/mL, 150 ng/mL, 200 ng/mL, 250 ng/ml, 300 ng/ml, 350 ng/mL, 400 ng/mL, 500 ng/mL, 600 ng/mL, 700 ng/mL, 800 ng/mL, 900 ng/mL, 1000 ng/mL, 1500 ng/mL, or 2000 ng/mL.

The C_{steady-state} of the therapeutic peptide in serum produced by the composition can be within a range from 10 ng/mL to 2000 ng/mL, 10 ng/mL to 1000 ng/mL, 10 ng/mL to 500 ng/mL, 10 ng/ml to 100 ng/mL, 10 ng/mL to 50 ng/mL, 10 ng/ml to 25 ng/mL, 50 ng/ml to 2000 ng/mL, 50 ng/mL to 1000 ng/mL, 50 ng/mL to 500 ng/mL, 50 ng/mL to 100 ng/ml, 100 ng/mL to 2000 ng/mL, 100 ng/mL to 1000 ng/mL, 100 ng/mL to 500 ng/mL, 100 ng/ml to 300 ng/ml, 150 ng/mL to 250 ng/mL, 500 ng/mL to 2000 ng/mL, 500 ng/mL to 1000 ng/mL, or 1000 ng/ml to 2000 ng/mL.

The C_{steady-state} of the incretin mimetic or acylated peptide can be achieved within the first 1 day, 2 days, 3 days, 4 days, 5 days, 6 days, or 7 days of administering the composition to the subject. In some embodiments, when administered to a subject in vivo, the composition produces a maximum concentration (Cₘₐₓ) of the incretin mimetic or acylated peptide in serum that is less than or equal to 2000 ng/mL, 1500 ng/mL, 1000 ng/mL, or 500 ng/mL.

### IV. 3D Printing

The present disclosure provides methods for 3D printing using a hydrogel composition as described herein. In some embodiments, the hydrogel composition exhibits favorable mechanical properties, such as high extensibility, that allow for faster printing speeds and/or printing of smaller feature sizes. Moreover, the hydrogel composition can be used to encapsulate therapeutic agents, thereby allowing for 3D printing of objects containing such therapeutic agents for biomedical applications such as tissue engineering, drug delivery, etc.

In some embodiments, a method for forming an object using a hydrogel composition is provided. For example, the object can be a tissue engineering scaffold, a drug delivery depot, a medical device or portion thereof (e.g., a layer, coating, etc., of a medical device), or a combination thereof. The object can include the hydrogel composition with a first agent (e.g., HPMC-C18) and a second agent (e.g., a surfactant or cyclic polysaccharide) as described herein. In some embodiments, the hydrogel composition includes a therapeutic agent, such as cells, proteins, peptides, nucleic acids, polysaccharides, or any of the other examples described herein. Alternatively, the hydrogel composition may not include any therapeutic agent (e.g., a therapeutic agent may be added to the object after 3D printing, or the object may be used without any therapeutic agents).

In some embodiments, a method for forming an object includes depositing a hydrogel composition on a surface using a 3D printing process. For example, the object can be formed using an extrusion-based 3D printing process in which the hydrogel composition is extruded from a nozzle at a controlled flow rate to form a continuous filament on a printing surface. The nozzle can be moved through a sequence of positions at a controlled movement speed to deposit the filament at specific locations on the printing surface to form the desired object geometry. In some embodiments, the hydrogel compositions herein have sufficiently high extensibility to be compatible with an extrusion-based process, e.g., the filament of the hydrogel composition is unlikely to break under the forces and conditions of the extrusion-based process. For instance, the hydrogel composition can have an extensional strain to break of at least 500%, 1000%, 1500%, 2000%, or 2500%, e.g., measured using a parallel plate rheometer at 25° C. and a strain rate within a range from 0.06 s⁻¹ to 0.3 s⁻¹. High extensibility can be achieved, for example, by increasing the concentration of the second agent (e.g., Tween 80) present in the hydrogel composition. For example, the concentration of the second agent can be at least 0.5 wt %, 0.75 wt %, 1 wt %, 1.5 wt %, or 2 wt %; and/or within a range from 0.5 wt % to 2 wt %, 0.5 wt % to 1.5 wt %, 0.5 wt % to 1 wt %, 0.5 wt % to 0.75 wt %, 0.75 wt % to 2 wt %, 0.75 wt % to 1.5 wt %, 0.75 wt % to 1 wt %, 1 wt % to 2 wt %, 1 wt % to 1.5 wt %, or 1.5 wt % to 2 wt %.

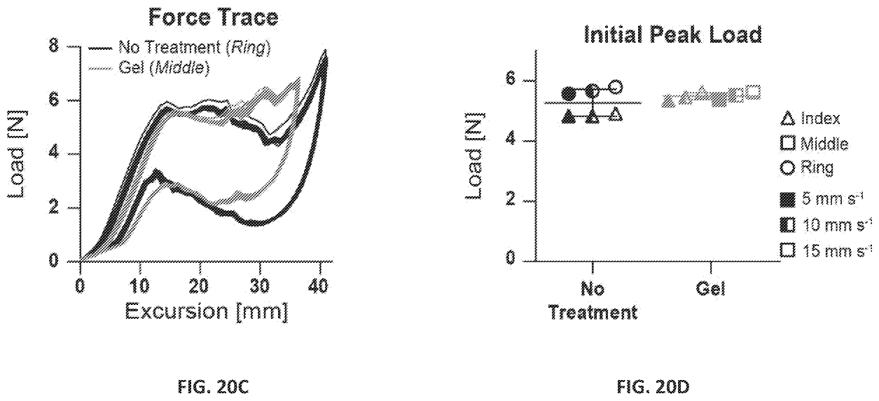

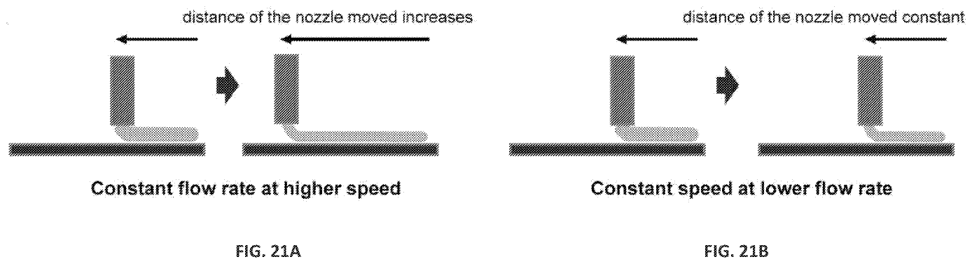

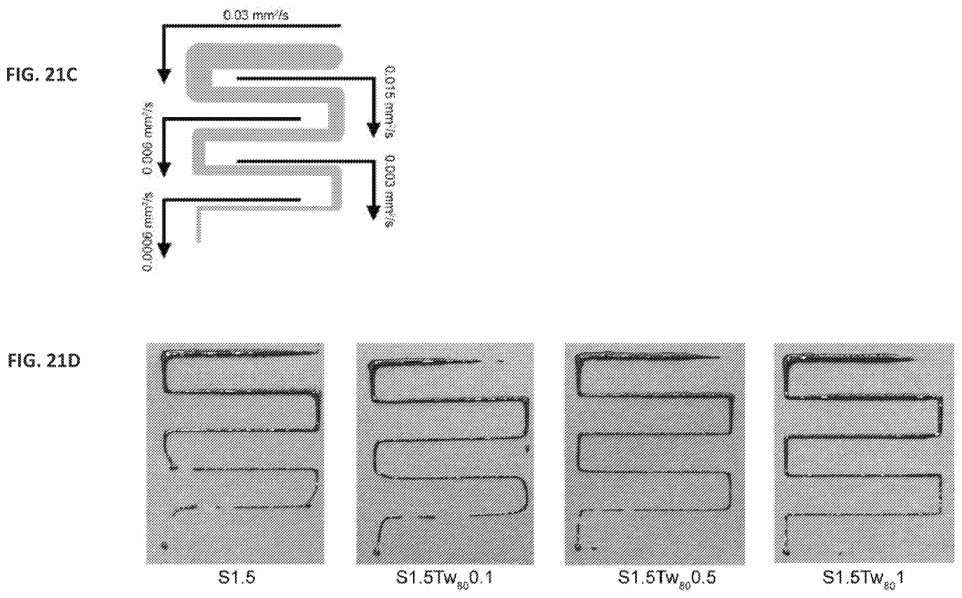

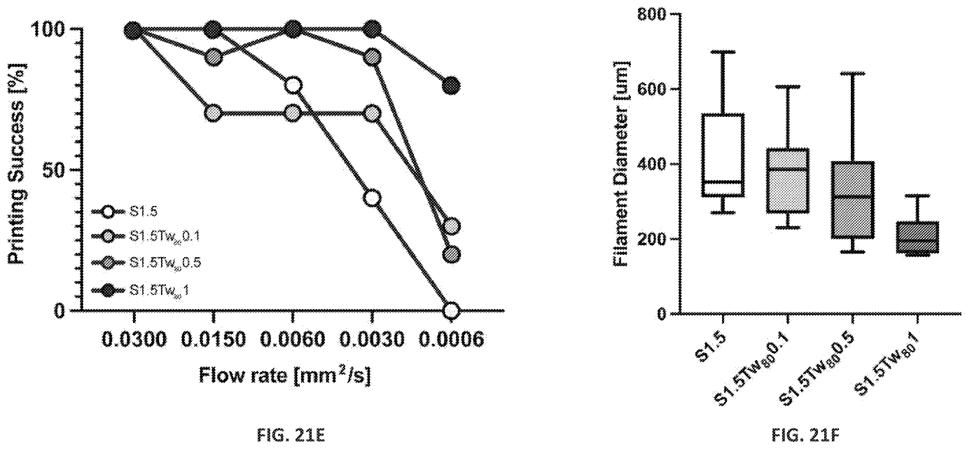

Optionally, the high extensibility of the hydrogel composition can allow for deposition of filaments having the same or a smaller diameter than the diameter of the nozzle, which may be challenging to achieve using conventional compositions due to the die swell effect. For example, the nozzle diameter can be at least 100 μm, 200 μm, 300 μm, 400 μm, 500 μm, 600 μm, 700 μm, 800 μm, 900 μm, 1 mm, or 2 mm; and/or the filament diameter can be no more than 2 mm, 1 mm, 900 μm, 800 μm, 700 μm, 600 μm, 500 μm, 400 μm, 300 μm, 200 μm, 100 μm, 50 μm, 20 μm, or 10 μm. A filament diameter smaller than the nozzle diameter can be achieved by stretching the filament after it is extruded from the nozzle. The stretching can increase the length of the filament, thus resulting in a reduction of the diameter of the filament. In some embodiments, stretching of the filament is achieved by independently controlling the flow rate of the filament from the nozzle and/or the movement speed of the nozzle relative to the printing surface. For example, FIG. 21A is a side-view schematic illustration of extrusion of a filament from a nozzle at a constant flow rate while increasing the lateral movement speed of the nozzle, thereby causing the extruded filament to stretch to a longer length with a smaller diameter. As another example, FIG. 21B is a side-view schematic illustration of extrusion of a filament from a nozzle at a lowered flow rate while maintaining a constant lateral movement speed of the nozzle, thereby causing the extruded filament to stretch to a longer length with a smaller diameter. This approach can be used to print objects with smaller feature sizes than can be achieved with conventional extrusion-based processes.

In some embodiments, a hydrogel composition for 3D printing includes a first agent and a second agent, wherein the first agent is a hydrophobically modified polysaccharide (e.g., HPMC-C18), and the second agent is a surfactant (e.g., Tween 20 or Tween 80) or a cyclic polysaccharide (e.g., cyclodextrin). In some embodiments, the concentration of the first agent is about 1.5 wt % and the second agent is at least about 1 wt %. In some embodiments, the concentration of the first agent is about 1.5 wt % and the second agent is at least about 1 wt %, wherein the first agent is HPMC-C18, and the second agent is Tween 80. In some embodiments, the concentration of the first agent is about 1.5 wt % and the second agent is about 1 wt %, wherein the first agent is HPMC-C18, and the second agent is Tween 80.

In some embodiments, the concentration of the first agent is about 1.5 wt % and the second agent is at least about 0.5 wt %, wherein the first agent is a hydrophobically modified polysaccharide (e.g., HPMC-C18); and the second agent is a surfactant (e.g., Tween 20 or Tween 80) or a cyclic polysaccharide (e.g., cyclodextrin). In some embodiments, the concentration of the first agent is about 1.5 wt % and the second agent is at least about 0.5 wt %, wherein the first agent is HPMC-C18, and the second agent is Tween 80. In some embodiments, the concentration of the first agent is about 1.5 wt % and the second agent is about 0.5 wt %, wherein the first agent is HPMC-C18, and the second agent is Tween 80.

Optionally, a hydrogel composition for 3D printing can include one or more reactive species (e.g., monomers, oligomers, reactive polymers, photoinitiators, thermal initiators) that polymerize and/or crosslink upon exposure to energy (e.g., heat, light) to form a covalently crosslinked polymer network. The formation of a covalently crosslinked polymer network can stabilize the object structure and/or enhance the mechanical properties (e.g., strength) of the object. In such embodiments, the object can be formed by depositing the hydrogel composition with the reactive species onto the printing surface (e.g., as a continuous filament), and applying energy to the hydrogel composition to polymerize the reactive species. For example, the hydrogel composition can be deposited to form a layer of the object, then the energy can be applied to polymerize the reactive species within the layer, then the hydrogel composition can be deposited on the polymerized layer to form a subsequent layer of the object, etc., until the entire object geometry is formed. In other embodiments, however, a hydrogel composition can be used for 3D printing without any reactive species and/or without forming a covalently crosslinked polymer network.

### V. Conclusion

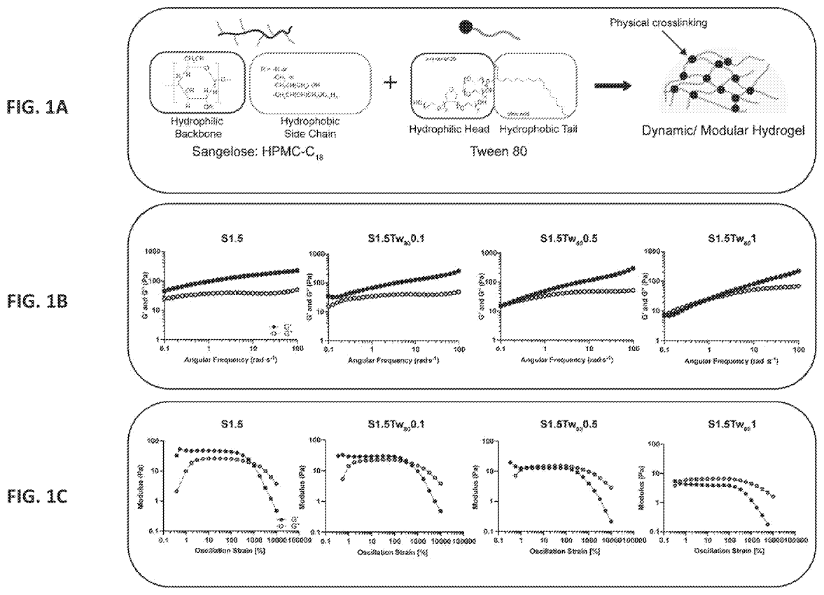

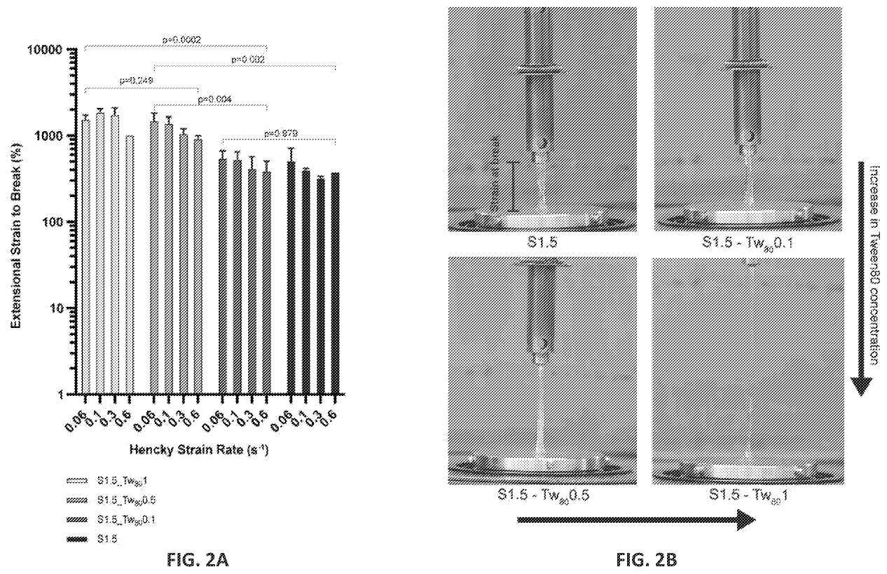

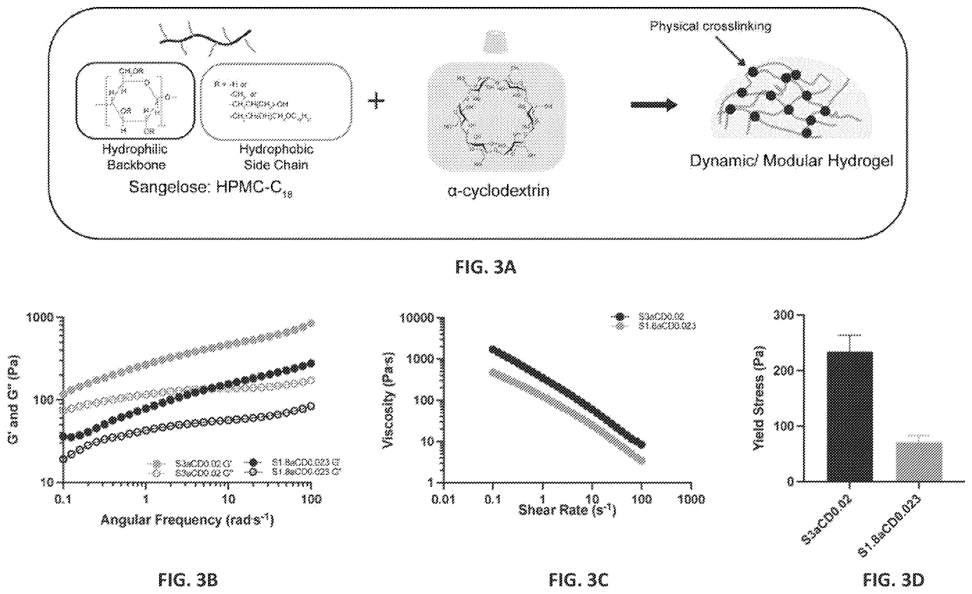

Although many of the embodiments are described above with respect to compositions and methods relating to drug delivery systems, therapeutic treatment of individuals, and 3D printing, the technology is applicable to other applications and/or other approaches, such as gene therapy applications. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-21F.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

### VI. Examples

The present technology is further illustrated by the following non-limiting examples.

### Example 1: Examples of Hydrogel Compositions, Rheological Studies, and Vaccine Delivery

This example describes preparation and characterization of hydrogel compositions.

A hydrogel is a crosslinked hydrophilic polymer network that can absorb high content of water (over 90% of the total weight). Hydrogels are useful in various biomedical-related applications due to their unique properties, such as high water content, biocompatibility, and injectability. A physically crosslinked hydrogel network may have tunable characteristics with reversible crosslinks that react to external stress such as temperature, pressure, and/or stress. Such properties may give rise to shear-thinning behavior—the viscosity of the material decreases under shear stress—that enables the hydrogel network to be injectable. Reversible crosslinking may allow the hydrogel network to self-heal once the shear stress is removed. These distinctive features, as well as biodegradability and biocompatibility through high water content, motivated in the investigation of hydrogels for forming a drug cargo niche post-injection for sustained drug delivery.

Sangelose 90L (Daido Chemical Corporation) is a hydrophobically-modified hydroxymethyl cellulose with a C18 stearyl group side chain. The polymer is a white powder that forms a water-swollen polymer network (hydrogel) when dissolved in water with high water content (over 90 wt % water). Sangelose can provide a cost-effective and facile hydrogel network with shear-thinning and self-healing behavior, which may be important characteristics of an injectable hydrogel system.

The rheological properties of Sangelose hydrogels can be easily tuned when mixed with surfactants such as Tween 20 or Tween 80, or with αCD. Oscillatory frequency sweeps of Sangelose-only hydrogels showed long relaxation times, and flat plateaus of storage and loss modulus. Following addition of αCD or Tween surfactants, the crossover point between storage and loss modulus shifted to the right, thus creating a hydrogel with more dynamic properties. Results of material characterization of Sangelose hydrogels with Tween 80, αCD, and Tween 20 are shown in FIGS. 1A-4F, respectively. Some of the procedures used herein were performed according to the techniques described in U.S. Patent Publication No. 2017/0319506, the disclosure of which is incorporated herein by reference in its entirety. The results indicate that hydrogel properties were easily tunable by simply controlling the weight percent of Sangelose and the surfactants or αCD. These Sangelose hydrogels with Tween or αCD can be easily formed by simply mixing Sangelose powder with water that contains either the surfactant or αCD.

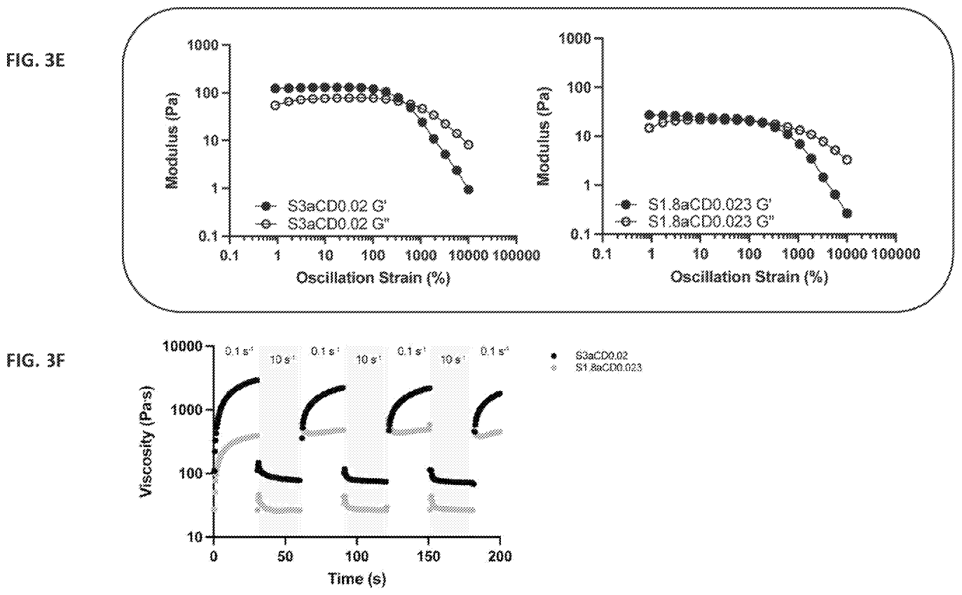

A dynamic hydrogel network achieved with the Tween surfactant and αCD can allow higher cell infiltration in vivo and can serve as good drug or vaccine cargo for sustained delivery. FIGS. 5A-5C shows the results from an in vivo vaccination study with gE protein as antigen and Cyclic GMP-AMP (cgamp) as an adjuvant under subcutaneous injection. These results show that Sangelose with Tween 20 showed the most robust and stable antibody response for sustained delivery.

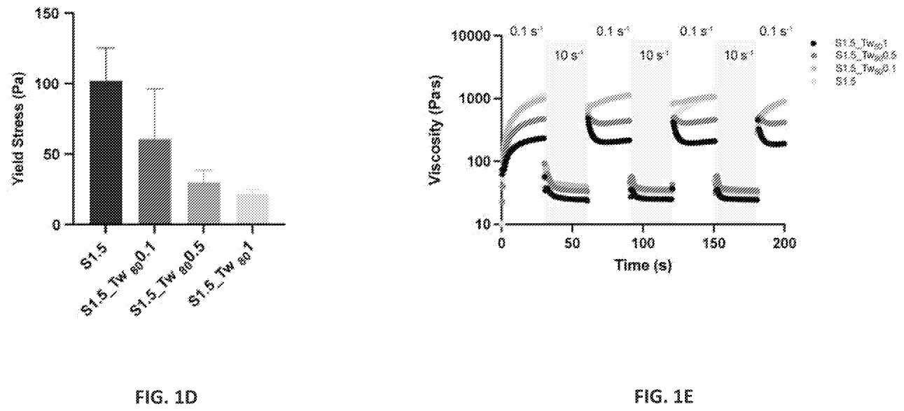

Sangelose and Tween 80 hydrogels can exhibit the distinctive feature of extensibility with low yield stress. FIGS. 2A and 2B show that the hydrogel extensibility can be controlled by Tween 80 concentration, with increasing Tween 80 concentration resulting in increased strain to break.

The hydrogels described herein may be suitable for use in 3D printing. The high extensibility of the hydrogel material suggests that the material can be printed at high speeds without disconnection. Hydrogel material properties, such as yield stress, flow characteristics, and/or extensibility, can also be easily tuned by changing the concentration of the Sangelose or Tween 80 surfactant. It is expected that cell viability can be maintained in the 3D printed hydrogel material, thus indicating its potential for use in the biomedical 3D printing field.

Sangelose with Tween 20 and αCD can improve vaccine performance through sustained delivery of vaccine components. The hydrogel can serve as an in vivo niche for drug cargo that can deliver drug components through slower diffusion compared to bolus injection. Sustained delivery of the drug cargo can improve humoral immunity through prolonged exposure of the drug. Sangelose hydrogels combined with Tween 20 or αCD are easily manufacturable and easily tunable, and thus can be a simple yet effective solution to improve conventional vaccines by changing the delivery method.

The hydrogels described herein can outperform existing hydrogel methods in ease of production, cost-effectiveness, and short preparation time. These advantages are ideal for mass production of the product and commercialization, among other benefits.

Further directions for investigation may include in vivo experimentation to determine the appropriate hydrogel composition for proper bolus control; hydrogel characterization after implantation to study cell infiltration behavior; scaling up of hydrogel preparation to determine whether the product is manufacturable in greater scale; and/or evaluation of printability with different pressures, printing speeds, and/or nozzle sizes.

### Example 2: Preparation of Unloaded Hydrogel Compositions

This example describes a process for preparing hydrogel compositions comprising hydrophobically modified hydroxypropyl methylcellulose (e.g., HPMC-C18) as a first agent, and a second agent (e.g., Tween 80, Tween 20, Tween 40, Tween 60, αCD, and Span 20).

The first agent, hydrophobically modified hydroxypropyl methylcellulose (e.g., HPMC-C18), also known by the tradename Sangelose, was dissolved in phosphate-buffered saline at 6 wt % and loaded into a 1.5 mL Eppendorf tube. The second agent (Tween 80, Tween 20, Tween 40, Tween 60, αCD, or Span 20) was dissolved in either phosphate-buffered saline or ethanol, depending on the solubility of the second agent, then added to the Sangelose solution. The contents were mixed thoroughly in the Eppendorf tube using a long spatula until homogeneous. The tubes were placed on a tabletop centrifuge for 5 minutes to remove bubbles, then stored at 25° C. overnight before testing. All hydrogel compositions were composed of Sangelose and a second agent in phosphate-buffered saline unless otherwise specified.

Table 2 lists the hydrogel compositions prepared using the above method.

TABLE 2

| Sangelose | Second | Wt % of Second |
|---|---|---|
| (wt %) | Agent | Agent |
| 3 | Tween 80 | 0.1, 0.5, 0.75, 1, 2 |
| 1.5 | Tween 80 | 0.1, 0.5, 1 |
| 3 | Tween 20 | 0.1, 0.75, 1, 2 |
| 3 | Tween 40 | 0.75 |
| 3 | Tween 60 | 0.75 |
| 3 | αCD | 0.05 |
| 3 | Span 20 | 0.75 |

The hydrogel compositions herein may be referred to in an abbreviated form. For instance, the weight percent of each component is noted after the component name. For instance, S3Tw₂₀0.75 refers to the hydrogel formulation with 3 wt % of Sangelose and 0.75 wt % of Tween 20. Abbreviations: Sangelose (HPMC-C18)=S; Tween 80=Tw₈₀; Tween 60=Tw₆₀; Tween 40=Tw₄₀; Tween 20=Tw₂₀; Span 20=Span₂₀; α-cyclodextrin=αCD or αCD.

### Example 3: Rheological Characterization of Hydrogel Compositions

This example describes methods to measure rheological properties of the hydrogel compositions described in Example 2 as well as Sangelose compositions without a second agent.

Rheological characterization was performed using a 20-mm diameter serrated parallel plate at a 500-μm gap on a TA Instruments DHR-2 stress-controlled rheometer. All measurements were performed at 25° C. Frequency sweep measurements were performed at a constant 1% strain. Steady shear flow sweeps were performed from high to low shear rates. Stress sweeps were performed from low to high with steady-state sensing and yield stress values defined as the stress at which the viscosity decreases 10% from the maximum. Amplitude sweep measurements were performed at a frequency of 0.1 rad·s⁻¹. P values are calculated with a one-way ANOVA followed by post hoc Tukey multiple comparison test unless otherwise noted.

Extensional rheology was performed on a TA Instruments ARES-G2 rheometer in axial mode using an 8-mm parallel plate geometry (R=4 mm) at a H=4 mm gap, resulting in an aspect ratio of H/R=1. A sample volume of 400 μl was loaded for each of the three replicated measurements. All experiments were performed at 25° C. Samples of the hydrogel compositions were loaded and tested immediately within seconds to minimize dehydration. Hencky (exponential) strain rates were applied at three different strain rates.

Sangelose-only compositions. Rheological measurements were taken of hydrogel compositions comprising Sangelose without a second agent with varying amounts of Sangelose (1.5 wt %, 2 wt %, 2.5 wt %, 3 wt %). FIG. 6B shows the results of frequency-dependent oscillatory shear sweep of the hydrogel samples. The crossover frequency (the frequency at which the storage modulus equals the loss modulus) was lower than the lowest angular frequency tested (0.1 rad·s⁻¹), which indicates that the hydrogels comprising only Sangelose have a static character (crossover frequency is inversely correlated to the relaxation time of the material; longer relaxation times correspond to more static materials, while shorter relaxation times correspond to more dynamic materials).

The storage modulus (G′) (FIG. 6C), and tan (delta) (FIG. 6D) was measured at 0.1 rad·s⁻¹ from FIG. 6B. In FIG. 6C, a decrease in Sangelose concentration was observed to correlate with a decrease in storage modulus (G′), which relates to the stiffness of the material (a lower storage modulus correlates to reduced stiffness). However, as shown in FIG. 6D, changes in Sangelose concentration did not significantly affect the tan (delta) values, which relate to the viscoelasticity of the material. This data demonstrates that changing the weight percent of Sangelose in a Sangelose only hydrogel can tune the stiffness but not the viscoelasticity of the gel.

Varying second agents. The frequency-dependent oscillatory shear rheology was measured for hydrogel compositions comprising Sangelose and various second agents: Tween 20 (FIG. 6E), Tween 80 (FIG. 6F), αCD (FIG. 6G), and Span 20 (FIG. 6H). The compositions tested include:
S3 (Sangelose only),
S3Tw₂₀0.75,
S3Tw₈₀0.75,
S3αCD0.05, and
S3Span₂₀0.75.

As shown in FIGS. 6E-6G, the addition of Tween 20, Tween 80, and αCD caused the crossover frequency (the frequency at which the storage modulus equals the loss modulus) of the hydrogel to shift rightward (toward higher angular frequencies) compared to the Sangelose only hydrogel. Accordingly, these results indicate that the addition of Tween 20, Tween 80, and αCD produced hydrogels with increased dynamic behavior compared to Sangelose only hydrogels. However, as shown in FIG. 6H, the addition of Span 20 did not change the crossover frequency of the hydrogel compared to the Sangelose only hydrogel, thus indicating that this second agent did not produce a dynamic hydrogel. Without being bound by theory, it is hypothesized that Tween 20, Tween 80, and αCD inserted into and created free volume within the micelles formed by the C18 side chains of the Sangelose polymer, thereby producing more dynamic crosslinks between the polymer chains. This effect may be due to chain length mismatch (e.g., Tween 20 has a saturated C12 tail which is shorter than the C18 side chain), presence of unsaturated bonds preventing tight packing (e.g., Tween 80 has an unsaturated C18 tail), and/or steric hindrance (e.g., Tween 20 and Tween 80 have large polyethoxylated sorbitan head groups, αCD is a sterically bulky molecule). In contrast, without being bound by theory, it is hypothesized that insertion of Span 20 into the micelles did not produce free volume due to reduced steric hindrance from its smaller sorbitan head group, such that the crosslinking between Sangelose polymer chains retained a relatively static character.

For each sample, the storage modulus (G′) and tan (delta) data was measured at 0.1 rad·s⁻¹ from FIGS. 6E-6H. As shown in FIG. 6I, the storage modulus of the hydrogels including Tween 20, Tween 80, and αCD was lower than the storage modulus of the Sangelose only hydrogels and hydrogels with Span 20. As shown in FIG. 6J, the tan (delta) values of the hydrogels including Tween 20, Tween 80, and αCD was higher than the tan (delta) values of the Sangelose only hydrogels and hydrogels with Span 20. These results demonstrate that varying the type of second agent influences both the stiffness and the viscoelasticity of the Sangelose-based hydrogel composition.

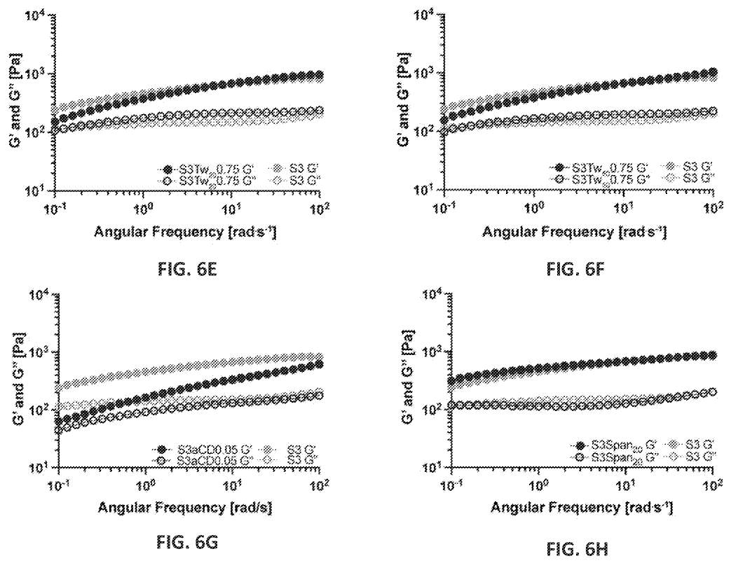

Comparison of Tween second agents. Additional Tween-type molecules were screened as second agents in hydrogel compositions. In addition to Tween 20 and Tween 80 discussed above, Tween 40, Tween 60, Tween 65, and Tween 85 were also used in hydrogel preparations. The Tween agents share the same hydrophilic head group. Their hydrophobic tails vary in chain length, saturation, and/or number, which can be seen in their chemical structure as shown in FIGS. 7A-7F. The hydrophobic tails shown are derived from lauric acid (C12, Tween 20), palmitic acid (C16, Tween 40), stearic acid (C18, Tween 60, Tween 65), and oleic acid (monounsaturated C18, Tween 80, Tween 85). The compositions tested include:
S3 (Sangelose only),
S3Tw₂₀0.75 (C12),
S3Tw₄₀0.75 (C16),
S3Tw₆₀0.75 (C18),
S3Tw₈₀0.75 (monounsaturated C18),
S3Tw₆₅0.75 (trisubstituted C18), and
S3Tw₈₅0.75 (trisubstituted monounsaturated C18).

The frequency-dependent oscillatory shear rheology was measured for hydrogel compositions and shown in FIGS. 7A-7F. A shift of the crossover point of G′ and G″ to the right was observed to correlate with decrease in hydrophobic tail length, as shown in FIGS. 7A-7C. A shift of the crossover point of G′ and G″ to the right was also observed to correlate with the introduction of an unsaturated site, as shown in FIGS. 7C and 7D. However, trisubstituted molecules did not significantly shift the crossover point compared to the Sangelose only hydrogel, as shown in FIGS. 7E and 7F, indicating that the hydrogels with trisubstituted molecules retained their relative static character. These results indicate that the chain length, saturation, and/or number of hydrophobic tails in Tween-type molecules affect their ability to produce dynamic behavior when combined with Sangelose.

The storage modulus G′ (FIG. 7G) and tan (delta) values (FIG. 7H) were measured at 0.1 rad·s⁻¹ from FIGS. 7A-7F. As shown in FIG. 7G, hydrogels formulated with Tween 20, Tween 40, and Tween 80 exhibited lower G′ values at a frequency of 10 rad·s⁻¹ compared to the hydrogels comprising only Sangelose and hydrogels formulated with Tween 80, Tween 65, and Tween 85. These data corroborate the increases in the observed crossover frequency described above as a shift of the crossover to higher frequencies results in a decrease in the observed modulus at a single low-to-intermediate frequency. As shown in FIG. 7H, hydrogels formulated with Tween 20, Tween 40, and Tween 80 exhibited significantly higher tan (delta) values compared to the hydrogels comprising Sangelose only, with the most significant change in tan (delta) value observed with Tween 20.

Varying the concentration of Tween 80. Hydrogel compositions comprising 3 wt % Sangelose and varying amounts of monounsaturated C18 Tween 80 (0.1 wt %, 0.5 wt %, 1 wt %, or 2 wt %) were prepared and the rheological measurements recorded. The compositions tested include:
S3 (Sangelose only),
S3Tw₈₀0.1,
S3Tw₈₀0.5,
S3Tw₈₀1, and
S3Tw₈₀2.

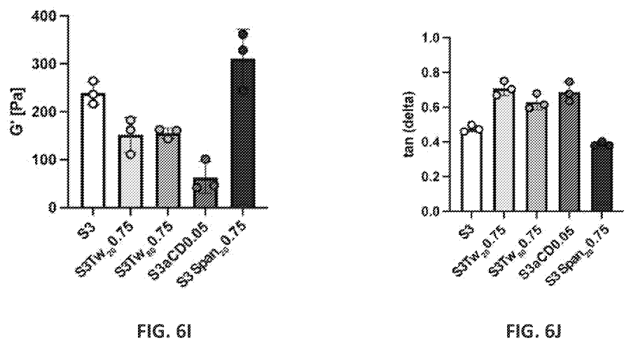

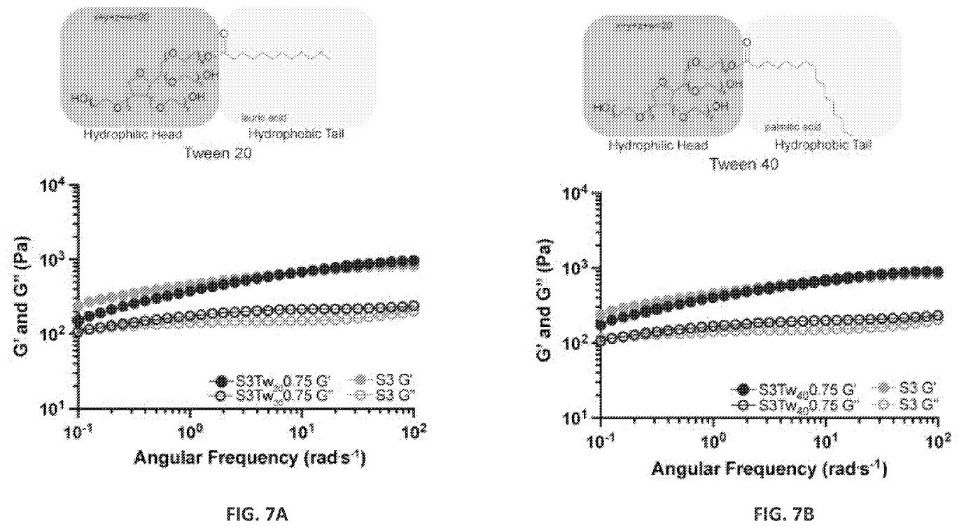

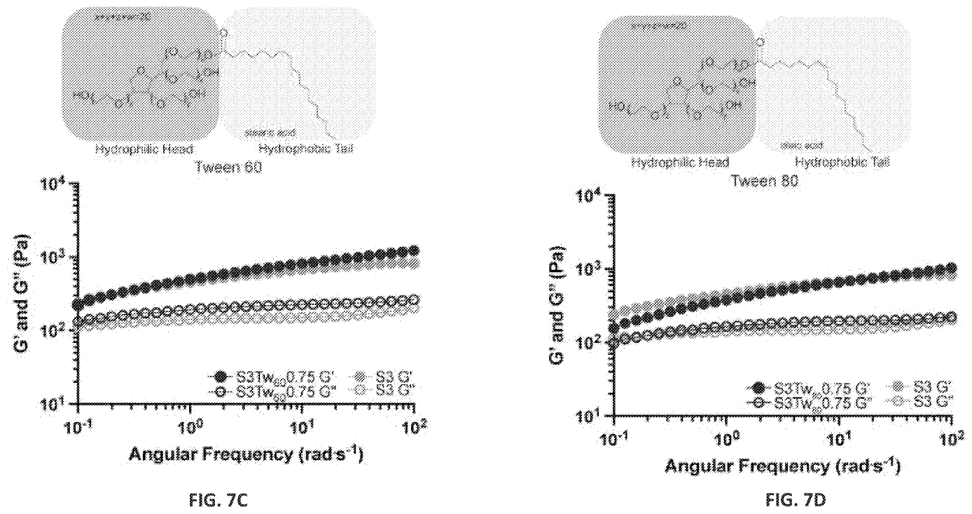

The frequency-dependent oscillatory shear sweep data of the four compositions are shown in FIG. 8A. A shift of the crossover point of G′ and G″ to the right was observed to correlate with increasing concentrations of Tween 80, thus indicating increased dynamic character of the hydrogels with higher Tween 80 concentrations.

The oscillatory amplitude sweep of the four compositions was measured at a frequency of 0.1 rad·s⁻¹ and depicted in the plots of FIG. 8B. The storage modulus G′ and tan (delta) values were measured at 0.1 rad·s⁻¹ and data shown in FIG. 8C and FIG. 8D, respectively. The storage modulus G′, which is an indicator of stiffness, was observed to decrease with increasing concentrations of Tween 80. The tan (delta), an indicator of viscoelasticity, generally increased with increasing concentrations of Tween 80. This data suggests that as the weight percent of the Tween 80 increases, the hydrogel becomes less stiff and more liquid-like.

Relaxation time of the hydrogel was calculated by fitting the frequency sweep with the continuous relaxation spectrum. FIG. 8E shows that the gel relaxation time decreased as the weight percent of the Tween 80 increased, thus indicating increased dynamic character of the hydrogels with higher Tween 80 concentrations.

Yield stress was determined through stress-controlled flow sweep with increasing stress. The plot of yield stress in FIG. 8F showed a general correlation between reduced yield stress and increased Tween 80 concentrations.

FIG. 8G is a plot of extensional strain data of hydrogel compositions comprising Sangelose and varying concentrations of Tween 80. FIG. 8H are representative images of hydrogel compositions comprising Sangelose and varying concentrations of Tween 80 under strain at a strain rate of 0.3 s⁻¹. As shown in FIGS. 8G and 8H, hydrogels with higher Tween 80 concentrations exhibited higher extensibility.

Varying the concentration of Tween 20. Similar to the experiment described above, studies were conducted to investigate the effect of increasing the concentration of Tween 20 on the rheology of the hydrogel compositions. Hydrogel compositions comprising 3 wt % Sangelose and varying amounts of monounsaturated C12 Tween 20 (0.1 wt %, 0.75 wt %, 1 wt %, or 2 wt %) were prepared and the rheological measurements recorded. The compositions tested were:
S3 (Sangelose only),
S3Tw₂₀0.1,
S3Tw₂₀0.75,
S3Tw₂₀1, and
S3Tw₂₀2.

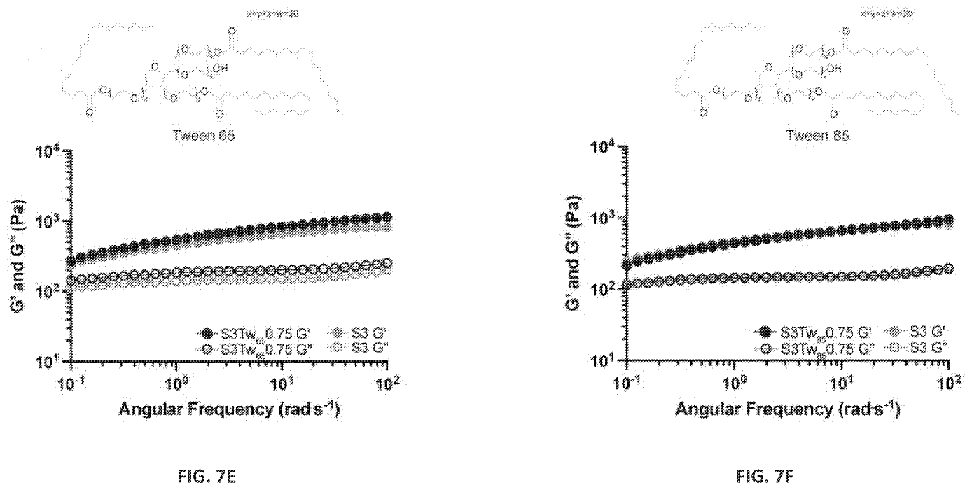

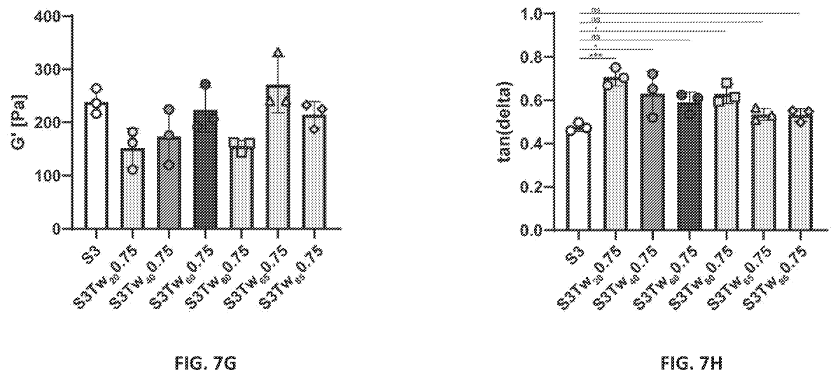

The frequency-dependent oscillatory shear sweep data of the four compositions are shown in FIG. 9A. A shift of the crossover of G′ and G″ to the right is observed to correlate with increasing concentrations of Tween 20, thus indicating increased dynamic character of the hydrogels with higher Tween 20 concentrations.

The oscillatory amplitude sweep of the four compositions was measured at a frequency of 0.1 rad·s⁻¹ and depicted in the plots of FIG. 9B. The storage modulus G′ and tan (delta) values were measured at 0.1 rad·s⁻¹ and data shown in FIG. 9C and FIG. 9D, respectively. The storage modulus G′, which is an indicator of stiffness, was observed to decrease with increasing concentrations of Tween 20. The tan (delta), an indicator of viscoelasticity, generally increased with increasing concentrations of Tween 20. This data suggests that as the weight percent of the Tween 20 increases, the hydrogel becomes less stiff and more liquid-like.

Yield stress was measured through a stress-controlled flow sweep with increasing stress and is shown for each of the compositions in FIG. 9E. The yield stress was similar for compositions with 0.1 wt %, 0.75 wt %, and 1 wt % Tween 20. The S3Tw₂₀2 composition containing 2 wt % of Tween 20 had a lower yield stress than the other compositions.

FIGS. 9F and 9G are plots of rheological data for a Sangelose-Tween 20 hydrogel composition (S3Tw₂₀0.75) and a Sangelose only hydrogel (S3). The viscosity of S3Tw₂₀0.75 and S3 as a function of shear rate was measured and the data presented in a plot shown in FIG. 9F. Both hydrogels showed shear-dependent viscosity and displayed shear-thinning behavior. Step-shear measurements taken over three cycles of alternating low shear (0.1 rad·s⁻¹) and high shear (10 rad·s⁻¹) rates in 50 s steps are shown in FIG. 9G. These results indicate that both hydrogels displayed self-healing behavior.

### Example 4: Vaccine Delivery with Hydrogel Compositions

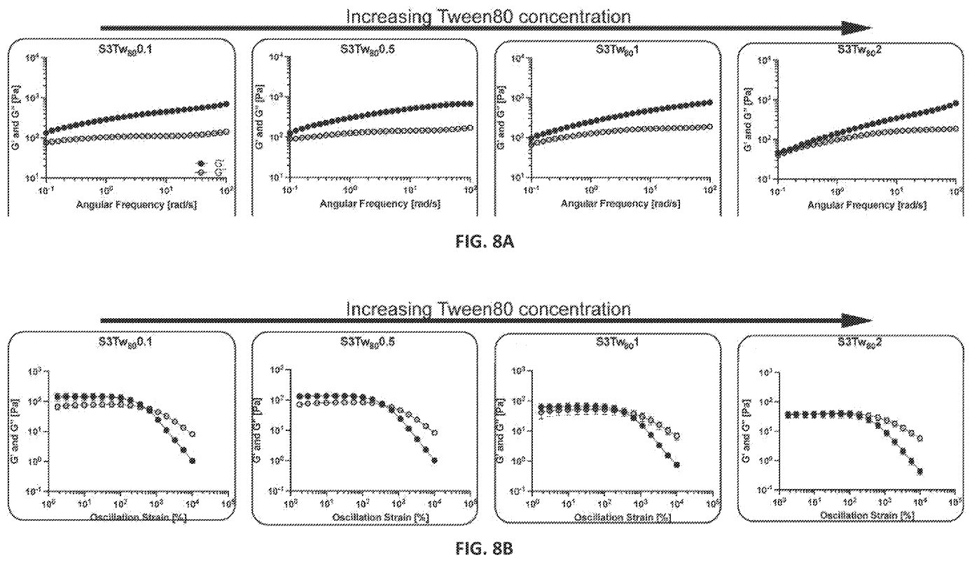

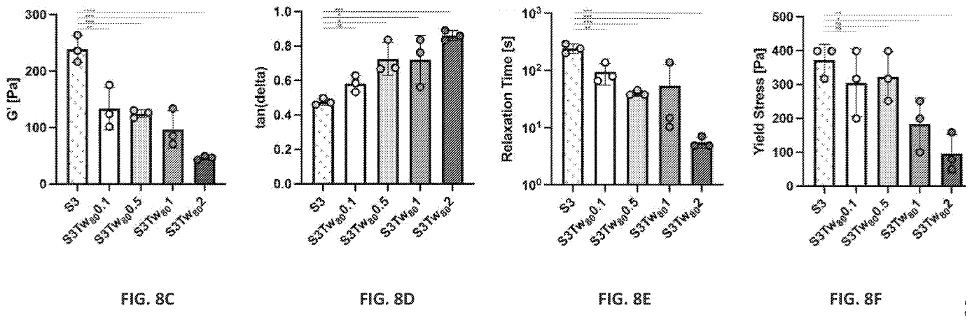

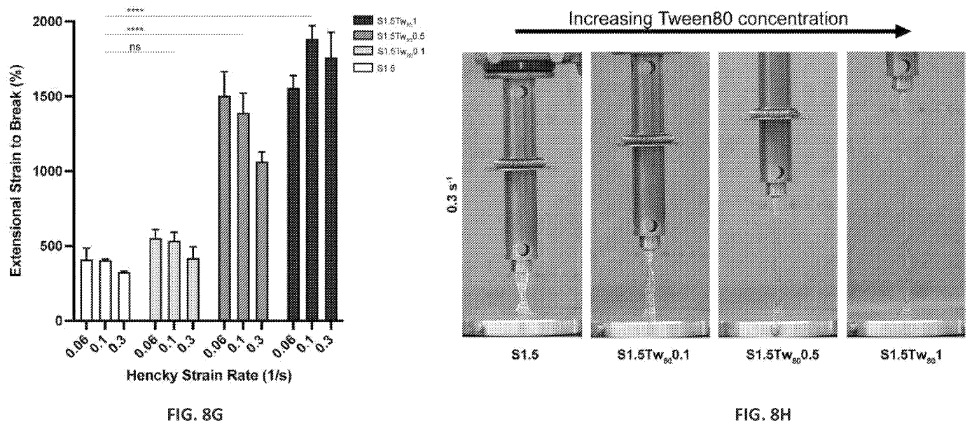

This example describes vaccination of mice with hydrogel compositions loaded with a shingles subunit vaccine and the observed humoral immune response. Briefly, subcutaneous injections of hydrogel or bolus formulations with 5 μg of gE antigen protein were given to mice, serum was collected weekly over time, and antigen specific IgG levels were measured. FIG. 10A is a schematic illustration of a timeline of mouse immunizations and blood collection.

Vaccine formulations. Compositions containing 5 μg of gE protein antigen (Shingrix) per dose were prepared in combination with varying concentrations of adjuvants in either bolus form (in PBS) or in hydrogel compositions (n=5). Adjuvants were dosed at:
10 μg or 20 μg per dose MPLAs (Invivogen, vac-mpls),
10 μg or 20 μg per dose AS01 for clinical trial control (Shingrix),
20 μg 2′3′-cGAMP (Invivogen, Vac-nacga23), or
2 μg of 3M-052 (3M and Advanced Health Institution, AAHI-AL030).

The following hydrogel compositions were prepared with the gE vaccine and adjuvant:
S2 (Sangelose only),
S3Tw₂₀0.75,
S1Tw₂₀0.25,
S2Tw₂₀0.5, and
S2Tw₂₀1.

The vaccine loaded hydrogel compositions was prepared by dissolving Sangelose in PBS at the corresponding weight percent and loaded into a 1-mL BD luer-lock syringe. The second agent, Tween 20, was added in the solution of PBS with adjuvant and gE protein antigen, and loaded into a second 1-mL syringe. The two syringes were connected with a female-female luer lock elbow, and gently mixed until a homogenous Sangelose hydrogel was formed.

Mice and vaccination method. All animal studies were performed in accordance with the National Institutes of Health (NIH) guidelines, with the approval of the Stanford Administrative Panel on Laboratory Animal Care. Eight-week-old C57BL/6 were purchased from Charles River and housed in the animal facility at Stanford University.

Mice were primed with 5 μg of gE protein in either bolus or in hydrogel, then boosted eight weeks later. All mice were injected subcutaneously on the right flank with 100 AL of bolus or hydrogel vaccine under brief isoflurane anesthesia. Blood was collected from tail veins weekly.

Antibody concentration ELISA. Antigen-specific IgG endpoint titers were measured using an endpoint ELISA. gE protein was coated to a 96-well ELISA plate at 1 μg/mL in PBS at 4° C. overnight and subsequently stored at −80° C. Plates were thawed for 1 hr at room temperature, washed, and blocked subsequently with PBS 1× with 1% bovine serum albumin (BSA) for 1 hr at 25° C. on a rotator. All wash steps included washing 5 times with 300 μL per well of wash solution (PBS with 0.05% Tween 20). Serum dilutions were prepared in a conical bottom plate (Thermo Scientific, 249570) in diluent buffer (PBS 1× with 1% BSA). Serum was first diluted at 1:100 or 1:200 (1 μL serum into 99 μL diluent buffer or 0.5 μL serum into 99.5 μL diluent buffer) and was serially diluted 4-fold or 8-fold (25 or 12.5 μL into 75 or 87.5 μL). Serum dilutions were transferred to antigen-coated plates after blocking and washing, 50 μL per well, and incubated for 2 hr at 25° C. Goat anti-mouse IgG Fc HRP (1:10,000, Invitrogen, A16084) was added at 50 μL per well and incubated for 1 hr. Plates were developed for 5 minutes with TMB substrate (Abcam, High Sensitivity, ab171523). The reaction was stopped with 1 N HCl. The plates were read at 450 nm with a Synergy H1 microplate reader (BioTek Instruments). Serum titers were determined in GraphPad Prism by fitting the curve with a five point asymmetric sigmoidal curve. The endpoint titers were defined as the serum dilution value at which the absorbance reached 0.1 and was interpolated from the curve fits. Samples below 0.1 absorbance at 1:100 dilution were designated below the limit of detection, and the endpoint titer was defined as a dilution value of 100.

Sangelose-only vaccine with MPL. FIG. 10B shows plots of Anti-gE IgG endpoint titers at 4 and 8 weeks, and the area under curve (AUC) of anti-gE titers for an S2 vaccine and Bolus+AS01 (positive control), further containing 20 μg MPL as an adjuvant. The immune response produced by the Sangelose-only gel was comparable to that of the control. The control formulation is known to be exceptionally potent but also exhibits significant issues with reactogenicity. The Sangelose hydrogels herein demonstrate similar potency, but the use of a less reactogenic agent and slower delivery rate may improve tolerability.

Adjuvant screening using S3Tw₂₀0.75. Anti-gE IgG endpoint titers were measured at 4 and 8 weeks after administration of Sangelose-Tween 20 hydrogels with different clinically relevant adjuvant, as shown in FIG. 10C. The hydrogel composition comprised 3 wt % of Sangelose and 0.75 wt % of Tween 20. The adjuvants screened included 20 micrograms of MPL, 3M052, or cGAMP. The AUC of the endpoint anti-gE titers are plotted for each composition and also shown in FIG. 10C. The immune responses were comparable across the three different adjuvants, thus indicating that the hydrogels can act as a platform for different vaccine formulations.

Hydrogel formulation screening with MPL adjuvant. Hydrogel compositions comprising varying amounts of Sangelose and Tween 20 were screened as vaccine carriers using 10 μg MPL as an adjuvant, including S3Tw₂₀0.75, S1Tw₂₀0.25, S2Tw₂₀0.5, and S2Tw₂₀1. Anti-gE IgG endpoint titers and AUC values resulting from the different Sangelose hydrogel vaccine formulations at 4 and 8 weeks are plotted in FIG. 10D. The stiffer hydrogel formulation (S3Tw₂₀0.75), which is expected to produce longer term delivery of the vaccine compared to weaker hydrogel formulations, produced more potent immune responses.

### Example 5: Preparation of Hydrogel Compositions with GLP-1 RAs

This example describes the preparation of hydrogel compositions comprising a GLP-1 RAs (semaglutide or tirzepatide) and various amounts of Sangelose and Tween 20.

Sangelose was purchased from Daido Chemical Corporation and polysorbate 20 was purchased from Sigma-Aldrich; both were used as received. Glassware and stir bars were oven-dried at 180° C.

Hydrogel compositions contained either 1.8 wt %, 2 wt % or 3 wt % Sangelose and 0.5 wt %, 0.6 wt % or 0.4 wt % Tween 20, and are denoted SANG-1.8-0.5, SANG-2-0.6 and SANG-3-0.4. These hydrogels were made by mixing a weighted ratio of 4 wt % Sangelose solution and either a 2 wt % or 4 wt % Tween 20 solution, and PBS or water containing GLP-1 RAs (semaglutide, tirzepatide, or liraglutide). The Tween 20 and aqueous components were mixed using an elbow connector. After mixing, the elbow was replaced with a 21-gauge needle for injection.

Table 3 summarizes semaglutide containing hydrogel compositions and their corresponding ingredients.

TABLE 3

| | | | | Propylene | | Sodium | Sodium | Potassium |
|---|---|---|---|---|---|---|---|---|
| | Semaglutide | Sangelose | Tween 20 | Glycol | Phenol | Chloride | Phosphate | Phosphate |
| | (ug/mL) | (mg/mL) | (mg/mL) | (mg/mL) | (mg/mL) | (mg/mL) | (mg/mL) | (mg/mL) |
| SANG-1.8-0.5-S1 | 1800 | 18 | 5.50 | 18.9 | 7.425 | 8.79 | 2.694 | 0.141 |
| SANG-1.8-0.5-S1a | 1800 | 18 | 5.50 | 18.9 | 7.425 | 0.00 | 1.917 | 0.000 |
| SANG-2-0.6-S2 | 1800 | 20 | 3.30 | 18.9 | 7.425 | 0.00 | 1.917 | 0.000 |
| SANG-3-0.4-S3 | 1800 | 28.8 | 2.16 | 7.56 | 2.97 | 8.71 | 1.536 | 0.139 |

Table 4 summarizes tirzepatide Sangelose hydrogel formulations and their corresponding ingredients.

TABLE 4

| | | | | Propylene | Sodium | Sodium | Potassium |
|---|---|---|---|---|---|---|---|
| | Tirzepatide | Sangelose | Tween 20 | Glycol | Chloride | Phosphate | Phosphate |
| | (ug/mL) | (mg/mL) | (mg/mL) | (mg/mL) | (mg/mL) | (mg/mL) | (mg/mL) |
| SANG-1.8-0.5-T1 | 1800 | 18 | 5.5 | 0 | 10.02 | 0.99 | 0.14 |
| SANG-1.8-0.5-T2 | 1800 | 18 | 5.5 | 0 | 1.23 | 0.21 | 0.000 |
| SANG-2-0.6-T3 | 4500 | 20 | 3.30 | 18.9 | 1.23 | 0.21 | 0.000 |

Table 5 summarizes liraglutide Sangelose hydrogel formulations and their corresponding ingredients.

TABLE 5

| | | | | Propylene | | Sodium | Sodium | Potassium |
|---|---|---|---|---|---|---|---|---|
| | Liraglutide | Sangelose | Tween 20 | Glycol | Phenol | Chloride | Phosphate | Phosphate |
| | (ug/mL) | (mg/mL) | (mg/mL) | (mg/mL) | (mg/mL) | (mg/mL) | (mg/mL) | (mg/mL) |
| SANG-1.8-0.5-L1 | 1800 | 18 | 5.50 | 18.9 | 7.425 | 8.79 | 2.694 | 0.141 |
| SANG-2-0.6-L2 | 1800 | 20 | 3.30 | 18.9 | 7.425 | 0.00 | 1.917 | 0.000 |
| SANG-3-0.4-L3 | 1800 | 28.8 | 2.16 | 7.56 | 2.97 | 8.71 | 1.536 | 0.139 |

Rheological testing was performed at 25° C. using a 20-mm-diameter serrated parallel plate at a 600-μm gap on a stress-controlled TA Instruments DHR-2 rheometer. All experiments were performed at 25° C. Frequency sweeps were performed from 0.1 to 100 rad s⁻¹ with a constant oscillation strain within the linear viscoelastic regime (1%). Amplitude sweeps were performed at a constant angular frequency of 10 rad·s⁻¹ from 0.01% to 10000% strain with a gap height of 500 μm. Flow sweeps were performed from low to high stress with steady-state sensing. Steady shear experiments were performed by alternating between a low shear rate (0.1 s⁻¹) and high shear rate (10 s⁻¹) for 60 s each for three full cycles. Shear rate sweep experiments were performed from 10 to 0.001 s⁻¹. Stress controlled yield stress measurements (stress sweeps) were performed from low to high stress with steady-state sensing and 10 points per decade.

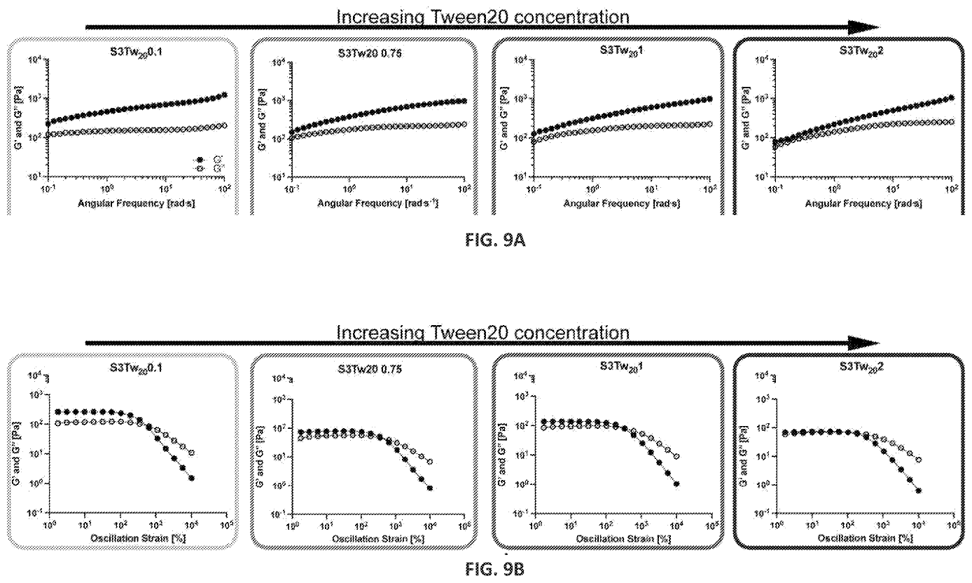

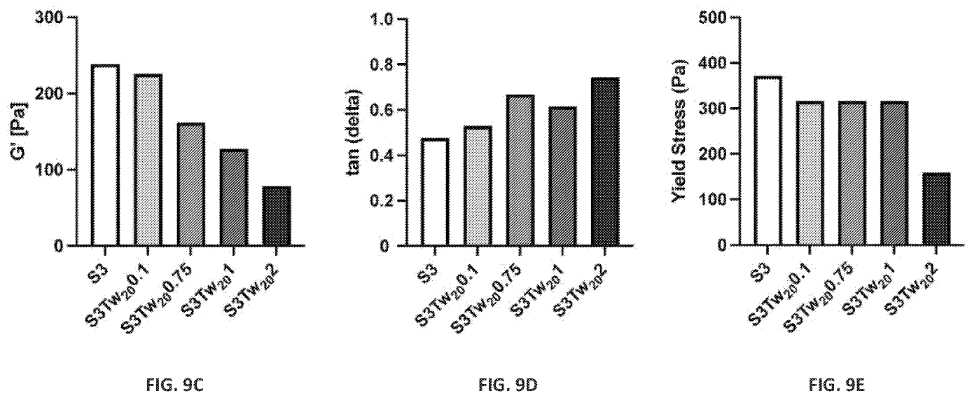

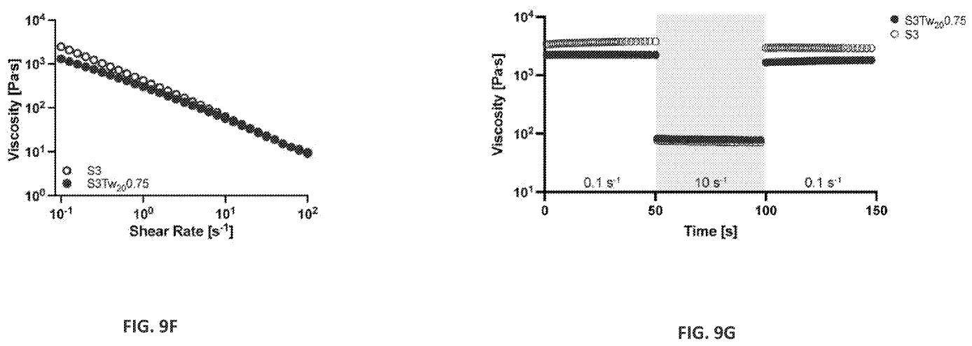

FIGS. 11A-11C are plots illustrating frequency-dependent oscillatory shear sweep rheological characterization of Sangelose hydrogel formulations without (SANG-2-0.6-no cargo) and with semaglutide cargo (SANG-2-0.6-sema 1.8 mg/mL, corresponding to SANG-2-0.6-S2 in Table 3) (FIG. 11A), without and with tirzepatide cargo (SANG-2-0.6-TZP 4.5 mg/mL, corresponding to SANG-2-0.6-T3 in Table 4) (FIG. 11B), and without and with liraglutide cargo (SANG-2.0-0.6-L 1.8 mg/mL, corresponding to SANG-2-0.6-L2 in Table 5) (FIG. 11C). The results indicate that incorporation of the semaglutide, tirzepatide, and liraglutide cargo did not significantly affect the rheological properties of the hydrogels.

### Example 6: In Vitro Release Profile of Semaglutide from Hydrogel Composition

This example describes an experiment to evaluate the in vitro release of semaglutide from hydrogel compositions discussed in Example 5 and listed in Table 3.

100 μL of each hydrogel formulation was loaded into four-inch capillaries and 400 μL of PBS medium was added slowly on top. The surrounding PBS was removed for analysis after 1, 3, 6, 12, 24, and 48 hours and at one week and two weeks after injection into the capillary, and fresh PBS was replaced after each aliquot removal. Semaglutide was quantified by ELISA to determine release kinetics over time.

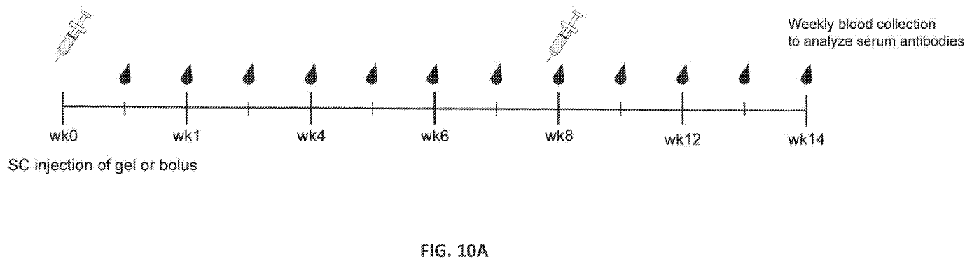

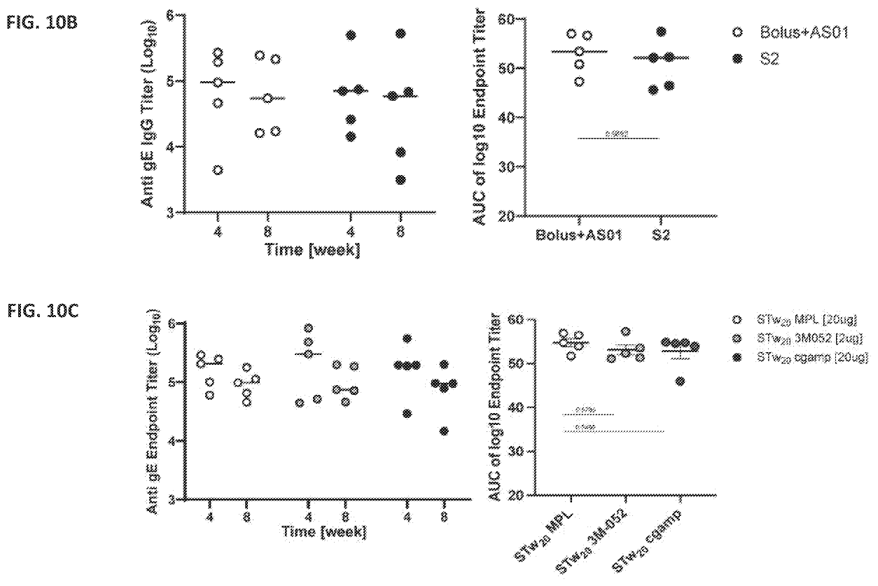

FIG. 12A is a plot of the % cumulative in vitro release of semaglutide over the course of one week from Sangelose-Tween 20 hydrogel compositions. These results show that all compositions exhibited low levels of burst release and high cargo retention over the one-week period, indicating successful encapsulation of semaglutide in the hydrogels. The SANG-2-0.6-S2 formulation was the most successful at slowing the release of the semaglutide cargo, and thus was used in the in vivo studies described in Example 8 below.

### Example 7: In Vitro Release Profile of Liraglutide from Hydrogel Composition

This example describes an experiment to evaluate the in vitro release of liraglutide from hydrogel compositions discussed in Example 5 and listed in Table 4.

FIG. 12B is a plot of the % cumulative in vitro release of liraglutide over the course of one week from Sangelose-Tween 20 hydrogel compositions. These results show that all compositions exhibited low levels of burst release and high cargo retention over the one-week period, indicating successful encapsulation of liraglutide in the hydrogels.

### Example 8: In Vivo Delivery of Semaglutide-Loaded Sangelose Hydrogel Composition

This example describes an experiment to evaluate in vivo delivery of semaglutide from a Sangelose hydrogel composition. A single dose of semaglutide-loaded Sangelose hydrogel composition was compared to a daily 20 μg semaglutide bolus injection and a daily PBS bolus injection over a period of six weeks in diabetic rats.

Animal model. Male Sprague Dawley rats 160-230 g (8-10 weeks, Charles River) were weighed and fasted in the morning 6-8 h prior to treatment with nicotinamide (NA) and streptozotocin (STZ). NA was dissolved in 1×PBS and administered intraperitoneally at 110 mg/kg. STZ was diluted to 10 mg/mL in sodium citrate buffer immediately before injection. STZ solution was injected intraperitoneally at 65 mg/kg into each rat. Rats were provided with water containing 10% sucrose for 24 h after injection with STZ. Rat blood glucose (BG) levels were tested for hyperglycemia daily after the STZ treatment via tail vein blood collection using a handheld blood glucose monitor (Bayer Contour Next). Type 2 diabetes (T2D) was defined as having three consecutive BG measurements in the range of 130-200 mg/dL in non-fasted rats.

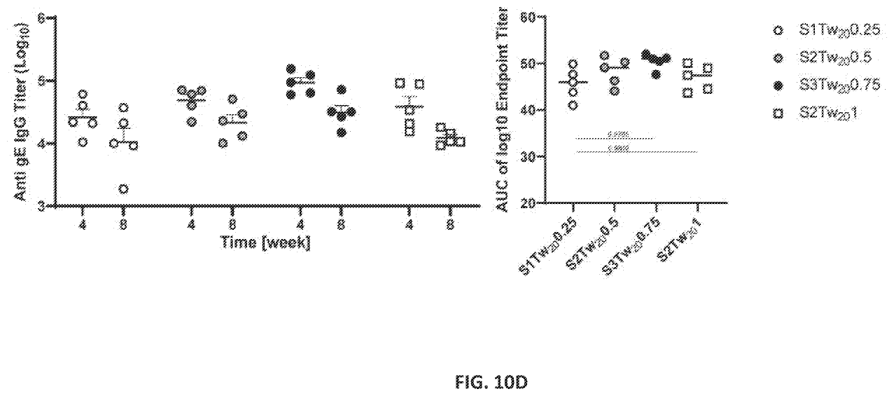

Semaglutide delivery and sample collection. Diabetic rats received either a) a single subcutaneous injection of a Sangelose hydrogel loaded with 1.8 mg/mL semaglutide (SANG-2-0.6-sema 1.86 mg/mL, corresponding to SANG-2-0.6-S2 in Table 3), or b) daily subcutaneous bolus injections of either PBS or 20 g semaglutide. Glucose measurements and serum collection were taken 5 days before injection of semaglutide and continued over a period of 6 weeks from injection. FIG. 13 is a schematic illustration of the treatment schedule and timing of blood glucose measurements and serum collection for analysis.

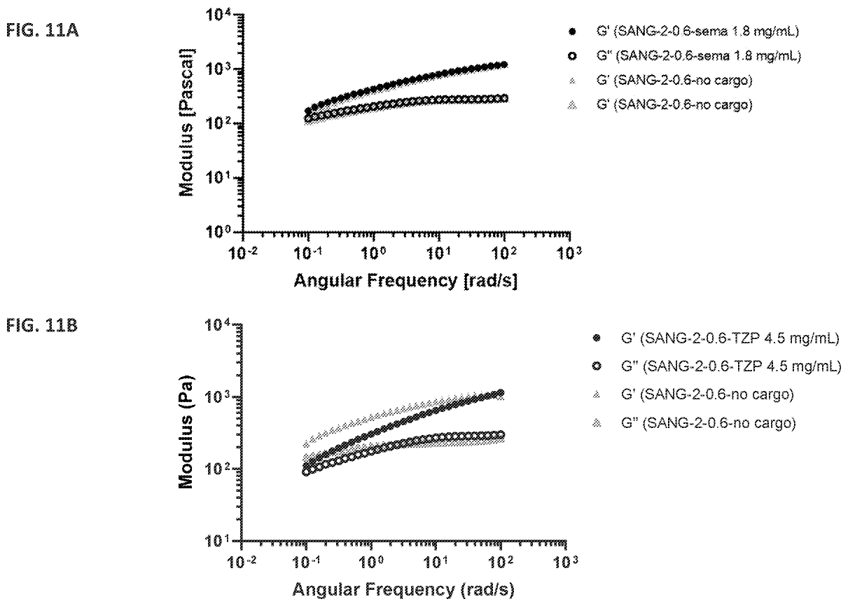

Oral glucose tolerance test. An oral glucose tolerance test (OGTT) was conducted in order to group the diabetic rats into separate treatment groups. Blood glucose was measured at −5, 0, 5, 15, 30, 45, 60, and 120 min. Using the area under the curve (AUC), rats with similar glucose tolerance were grouped and then randomized into treatment groups. FIG. 14 is a plot of data from an oral glucose tolerance test in rats, showing the area under the curve (AUC) of blood glucose after administration of a semaglutide bolus, semaglutide in a hydrogel composition, or PBS.

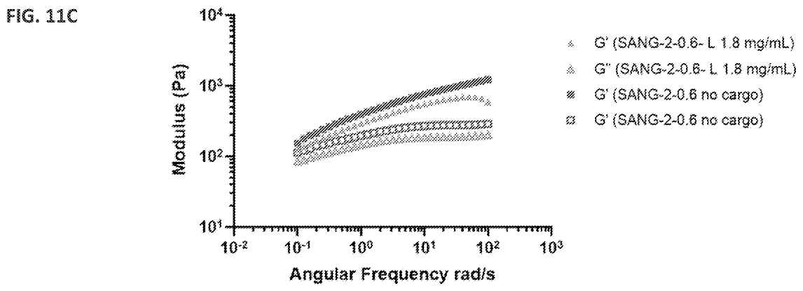

Blood glucose reduction. A single administration of the semaglutide-loaded Sangelose hydrogel reduced the blood glucose of Type 2-like diabetic male rats over the course of the first 5 days post treatment, compared to daily PBS bolus injections. FIG. 15 is a plot showing a change in blood glucose over 5 days following each treatment group regimen (n=6).

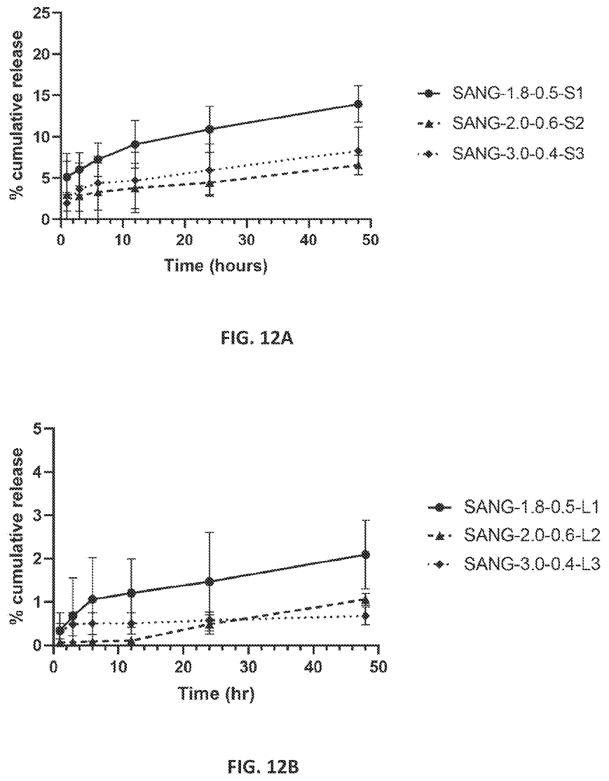

Weight reduction. A single administration of the semaglutide-loaded Sangelose hydrogel reduced the overall weight gain in Type 2-like diabetic male rats over the course of the first 5 days post treatment. FIG. 16 is a plot showing a change in weight over 5 days of each treatment group (n=6).

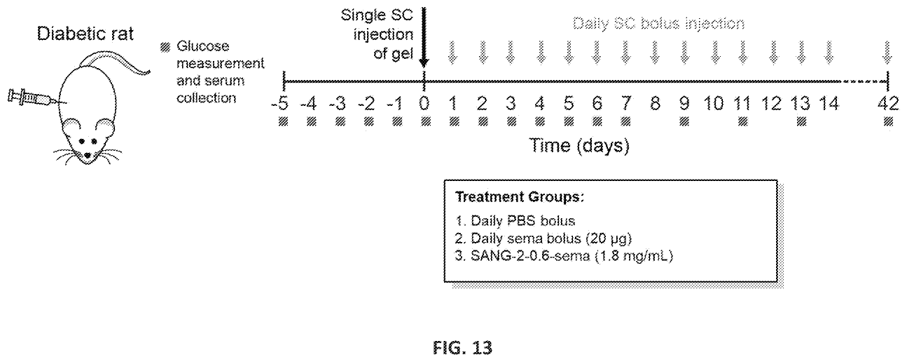

PK profile. The semaglutide serum concentration was measured daily by ELISA. FIG. 17 is a plot of semaglutide serum concentration in diabetic rats over the course of the first 5 days post administration of a single semaglutide hydrogel composition and daily bolus injections of 20 μg semaglutide. The hydrogel composition achieved comparable semaglutide serum levels as the daily bolus injections throughout the study period.

### Example 9: Adhesion Prevention with Hydrogel Composition

This example describes the evaluation of a hydrogel composition for adhesion prevention in a tendon glide experiment.

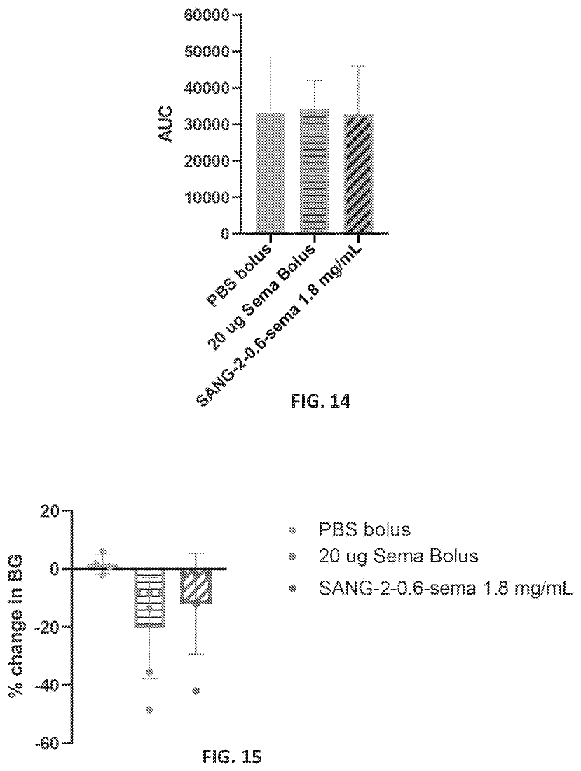

Standard of care treatment for a tendon injury is tendon repair, which often results in limited range of motion following healing due to adhesions around and between tendon tissue and other tissues. In this example, a hydrogel composition was applied following tendon surgery to act as a lubricious barrier and prevent adhesion formation. FIG. 18 is a schematic illustration of tendon repair with and without the application of a hydrogel composition.

Hydrogel rheological characterization. Rheological characterization was performed on Sangelose hydrogels (1.8 wt % Sangelose, 0.5% Tween 20) using a TA Instruments DHR-2 stress-controlled rheometer. All experiments were performed using a 20-mm diameter serrated plate geometry at 25° C. with a 500-μm gap. Stress sweeps were performed from low to high with steady state sensing and yield stress values were defined as the stress at which the viscosity decreases by 15% from the maximum pre-yield plateau value. For control runs, two serrated parallel plates were used. For tissue runs, cadaver skin was positioned in place of the bottom rheometer plate. Before loading hydrogels onto cadaver skin, sutures were sewn into the edges of the sample to act as anchors, which were then taped down to flatten the tissue under the geometry. Kimwipes were used to remove excess fluid from the surface of tissue samples and fatty protrusions were removed with a scalpel to further flatten the tissue surface.

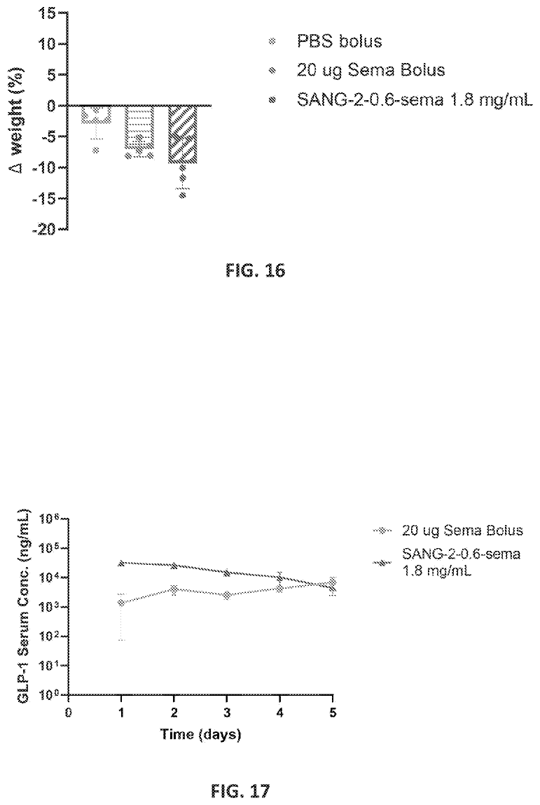

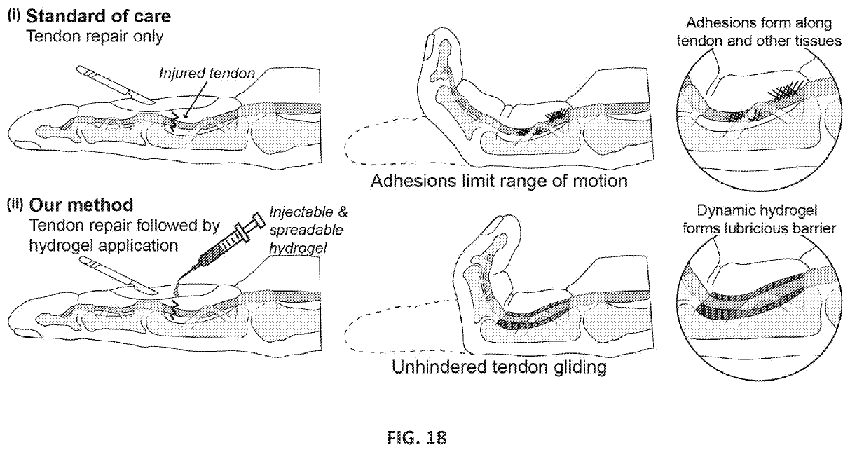

FIG. 19A is a schematic illustration of adhesive versus cohesive failure. Adhesive failure occurs at the tissue interface and would be measured as a lower yield stress due to slippage. Cohesive failure is a yielding of the hydrogel material and is indicated by equivalent yield stress of the material between parallel plates or between a plate and tissue. In contrast, adhesive failure occurs when the material slips on the tissue and is indicated by a lower yield stress between a plate and tissue than between parallel plates.

Representative images showing hydrogel loaded onto rheometer with cadaver skin tissue are shown in FIG. 19B. These images show a hydrogel loaded onto a rheometer plate in i) a 500-μm gap position and ii) a raised position following testing.

Rheology profile, yield behavior and yield stress results. Yield behavior was evaluated based on the viscosity of the hydrogel composition as a function of applied stress. A lower yield stress would suggest adhesive failure at the tissue interface due to slippage. Equivalent yield stress measurements of the material between parallel plates or between a plate and tissue suggests cohesive failure and a yielding of the hydrogel material. FIG. 19C is a plot of representative low to high flow sweep data of the hydrogel on plates to the hydrogel on skin. These results show hydrogels have the same yielding profile on tissue compared with the 20-mm serrated rheometer plate. Extracted yield stress values were calculated and defined as the stress at which the viscosity has decreased 15% from the pre-yield plateau value. Hydrogels yielded cohesively on standard rheometer plates as well as on cadaveric skin. FIG. 19D is a plot of this data shown as mean±SD, n=3. Overall, this oscillatory shear rheology data indicates that the Sangelose hydrogel has promising tissue adherent properties, and also yields cohesively under shear stress, resulting in a gliding effect between two tissues during movement. In this way, the hydrogel creates a lubricious layer between the tissues.

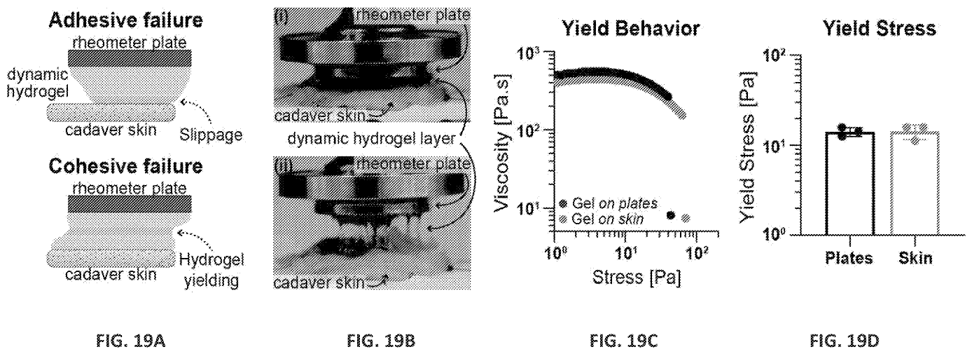

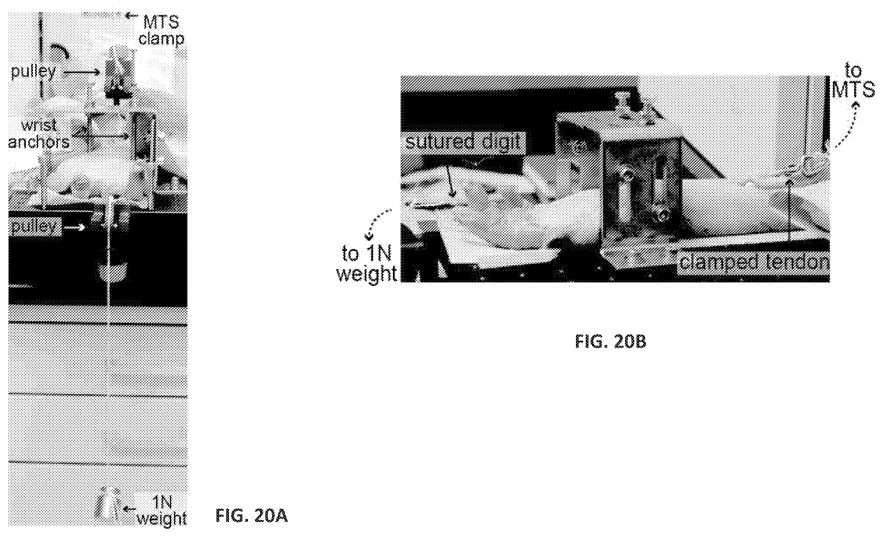

Glide testing of repaired tendons following material application. A custom rig was engineered to secure cadaver arms for testing glide friction of tendons following repair and hydrogel application. FIGS. 20A and 20B are representative images of the testing rig loaded with a cadaver arm. Cadaver arms were anchored to an optical table with bolts at the pisiform and scaphoid and a 100-g weight attached to each digit. Each tendon was attached to the test system with an alligator clamp and nylon rope and subjected to repeated cycles of 35-40 mm excursion.

To simulate injury, flexor digitorum profundus (FDP) tendons in the index, middle, and ring digits were isolated and severed between the A2 and A4 pulleys. Tendons were then repaired with a modified Kesler knot using 4-0 polypropylene sutures (Ethicon) and the skin was sutured closed. In treatment groups, 200-300 μL gel was injected on top of the repaired tendon and manipulated to coat the tendon before the skin was closed. An MTS Bionix 200 test system was used to apply displacement and measure force. The cadaver arm was positioned atop an optical table (Thorlabs) and secured with S-brackets in the form of an arch and bolts from the S-brackets to the pisiform and scaphoid wrist bones. An incision was made in the forearm to access the FDP tendons which were then cut, wrapped in sandpaper, and secured by an alligator clip at the proximal end. The alligator clip connected to a nylon string fed through a pulley and into the MTS clamp. Nylon string was tied to a suture loop on each fingertip, passed over a pulley, and attached to a 100-g counterweight to fully extended the fingers and allow testing of the whole range of motion during glide experiments. Force was tared and slack removed from the string and tendon before each experiment. Experiments were conducted to an excursion of 35-40 mm at speeds of 5, 10, and 15 mm s⁻¹. Bolts, brackets, nuts, alligator clips, and string were purchased from McMaster-Carr.

Results from glide testing of repaired tendons. FIG. 20C is a plot of force traces from digits tested at 10 mm s⁻¹. Curve differences following 20-mm excursion were inherent to the digit being tested and the initial peak load was used as a comparable parameter across digits. FIG. 20D is a plot of extracted initial peak load values for digits (index, middle, ring) treated with nothing or with hydrogel. Shapes denote which digit (index, middle, or ring) and shading of the shape denotes the testing speed (5, 10, or 15 mm s⁻¹). Data presented as mean±SD and each point is the mean of the speed trials (n=10, 30, 10 for 5, 10, 15 mm s⁻¹ respectively). This data suggests that hydrogel treatment of the repaired tendon does not impact peak load.

### Example 10: 3D Printing with Hydrogel Compositions

This example describes 3D printing using a Sangelose and Tween 80 hydrogel as a biocompatible bioink.

Compositions. Hydrogel compositions were prepared as described in Example 2, and comprised 1.5 wt % of Sangelose in combination with various amounts of Tween 80 (0.1 wt %, 0.5 wt %, 1 wt %) or without Tween 80. The compositions tested included:
S1.5 (Sangelose only),
S1.5Tw₈₀0.1,
S1.5Tw₈₀0.5, and
S1.5Tw₈₀1.

3D printing of hydrogel filaments. The hydrogel compositions were loaded in a BD 1 mL syringe on a 27G needle for printing. Syringe-loaded hydrogel bioink was loaded onto the mechanically controlled extrusion-based 3D printer. All samples were printed at a constant speed of 350 mm/min at varied flow rates (FIG. 21B).

FIG. 21C is an overhead schematic illustration of a printed filament at varying flow rates. This pattern, speed, and flow rate was followed in the printing process resulting in printed filaments shown in the representative images in FIG. 21D. The images show fewer breaks in the filament as the amount of Tween 80 increased from 0.1 wt % to 1 wt %. The Sangelose-only composition and the 0.1 wt % Tween 80 containing hydrogel had early filament breaks, while hydrogels with higher Tween 80 concentrations were able to maintain a continuous filament at thinner filament diameters. The printing success (whether the printed filament stayed together during printing, rather than breaking up) was shown as a function of flow rate as summarized in the plot in FIG. 21E. The minimum filament diameter before disconnection was measured and summarized in FIG. 21F. The hydrogel composition with the highest Tween 80 concentration (S1.5Tw₈₀1), which also had the highest extensional strain to break (FIG. 8H), had the highest printing success percentage and formed the narrowest filament diameter before breaking. This data suggests a correlation between increasing concentrations of Tween 80, hydrogel extensibility, and printing success.

### Example 11: Embodiments (I)

Embodiment I-1: A drug delivery system, comprising: a hydrogel composition comprising a mixture of: a hydrogel comprising a hydrophobically-modified hydroxypropyl methylcellulose with C18 stearyl group side chain; and a surfactant selected from Tween 20 or Tween 80, or alpha-cyclodextrin (αCD); wherein weight percentages of the hydrogel and surfactants or αCD are tuned to achieve a desired shear-thinning and self-healing behavior, and wherein a cargo comprising at least one therapeutic is encapsulated in the composition.
Embodiment 1-2: The Drug Delivery System of Embodiment I-1, Wherein the at Least One Therapeutic is an Immunomodulatory Composition
Embodiment I-3: The drug delivery system of claim Embodiment I-1, wherein the hydrogel composition is printed using a 3D printer.
Embodiment I-4: A method for delivering a drug to a patient comprising: injecting the patient with a hydrogel composition that encapsulates the drug, the hydrogel composition comprising a hydrophobically-modified hydroxypropyl methylcellulose with C18 stearyl group side chain and a surfactant selected from Tween 20 or Tween 80, or alpha-cyclodextrin (αCD), wherein weight percentages of the hydrogel and surfactants or αCD are tuned to achieve a desired shear-thinning and self-healing behavior.

### Example 12: Embodiments (II)

Embodiment II-1: A hydrogel composition, comprising: a first agent, wherein the first agent is octadecyl modified hydroxypropyl methylcellulose (HPMC-C18), and wherein the first agent forms micellar structures; and a second agent comprising a surfactant or cyclic polysaccharide, wherein the second agent disrupts the micellar structures of the first agent.
Embodiment II-2: The hydrogel composition of Embodiment II-1, wherein the second agent is a non-ionic surfactant.
Embodiment II-3: The hydrogel composition of Embodiment II-2, wherein the non-ionic surfactant has a hydrophilic-lipophilic balance (HLB) value of at least 9, at least 10, at least 11, at least 12, at least 13, at least 14, or at least 15.
Embodiment II-4: The hydrogel composition of Embodiment II-2 or Embodiment II-3, wherein the non-ionic surfactant is a polysorbate or a polyoxyethylene fatty ether.
Embodiment II-5: The hydrogel composition of Embodiment II-4, wherein the polysorbate is selected from the group consisting of polyoxyethylene (20) sorbitan monooleate (polysorbate 80), polyoxyethylene (20) sorbitan monolaurate (polysorbate 20), polyoxyethylene (20) sorbitan monopalmitate (polysorbate 40), and polyoxyethylene (20) sorbitan monostearate (polysorbate 60).
Embodiment II-6: The hydrogel composition of any one of Embodiments II-1 to II-5, wherein the second agent is polyoxyethylene (20) sorbitan monolaurate (polysorbate 20).
Embodiment II-7: The hydrogel composition of any one of Embodiments II-1 to II-6, wherein the second agent is polyoxyethylene (20) sorbitan monooleate (polysorbate 80).
Embodiment II-8: The hydrogel composition of Embodiment II-4, wherein the polyoxyethylene fatty ether comprises a hydrocarbon chain of 12 to 18 carbons and 2 to 100 oxyethylene groups.
Embodiment II-9: The hydrogel composition of Embodiment II-1, wherein the second agent is a cyclic polysaccharide.
Embodiment II-10: The hydrogel composition of Embodiment II-9, wherein the second agent is a hexasaccharide.
Embodiment II-11: The hydrogel composition of Embodiment II-9, wherein the second agent is α-cyclodextrin.
Embodiment II-12: The hydrogel composition of any one of Embodiments II-1 to II-11, wherein the micellar structures comprise C18 side chains of the HPMC-C18, and wherein the second agent is configured to noncovalently interact with the C18 side chains.
Embodiment II-13: The hydrogel composition of Embodiment II-12 wherein the second agent comprises a hydrophobic tail having a length different than a length of the C18 side chains of the HPMC-C18.
Embodiment II-14: The hydrogel composition of Embodiment II-12 or II-13, wherein the second agent comprises a hydrophobic tail having an unsaturated hydrocarbon group.
Embodiment II-15: The hydrogel composition of any one of Embodiments II-12 to II-14, wherein the second agent is configured to disrupt packing and/or organized assembly of the C18 side chains within the micellar structures.
Embodiment II-16: The hydrogel composition of any one of Embodiments II-12 to II-15, wherein the second agent is configured to create free volume between the C18 side chains within the micellar structures.
Embodiment II-17: The hydrogel composition of any one of Embodiments II-12 to II-16, wherein the second agent is dispersed within the micellar structures to form at least one mixed micelle.
Embodiment II-18: The hydrogel composition of any one of Embodiments II-12 to II-17, wherein the second agent binds to and prevents at least some of the C18 side chains from entering the micellar structures.
Embodiment II-19: The hydrogel composition of any one of Embodiments II-1 to II-18, wherein the concentration of the first agent is at least 0.1 wt %, at least 0.25 wt %, at least 0.5 wt %, at least 0.75 wt %, at least 1 wt %, at least 1.5 wt %, at least 2 wt %, at least 2.5 wt %, at least 3 wt %, at least 3.5 wt %, at least 4 wt %, at least 4.5 wt %, or at least 5 wt %.
Embodiment II-20: The hydrogel composition of any one of Embodiments II-1 to II-18, wherein the concentration of the first agent is 0.1 wt % to 5 wt %, 0.1 wt % to 4 wt %, 0.1 wt % to 3 wt %, 0.1 wt % to 2 wt %, 0.1 wt % to 1 wt %, 0.1 wt % to 0.5 wt %, 0.5 wt % to 5 wt %, 0.5 wt % to 4 wt %, 0.5 wt % to 3 wt %, 0.5 wt % to 2 wt %, 0.5 wt % to 1 wt %, 1 wt % to 5 wt %, 1 wt % to 4 wt %, 1 wt % to 3 wt %, 1 wt % to 2 wt %, 2 wt % to 5 wt %, 2 wt % to 4 wt %, 2 wt % to 3 wt %, 3 wt % to 5 wt %, 3 wt % to 4 wt %, or 4 wt % to 5 wt %.
Embodiment II-21: The hydrogel composition of any one of Embodiments II-1 to II-20, wherein the concentration of the second agent is at least 0.02 wt %, at least 0.1 wt %, at least 0.5 wt %, at least 1 wt %, at least 2 wt %, at least 3 wt %, at least 4 wt %, or at least 5 wt %.
Embodiment II-22: The hydrogel composition of any one of Embodiments II-1 to II-20, wherein the concentration of the second agent is 0.02 wt % to 5 wt %, 0.02 wt % to 4 wt %, 0.02 wt % to 3 wt %, 0.02 wt % to 2 wt %, 0.02 wt % to 1 wt %, 0.1 wt % to 5 wt %, 0.1 wt % to 4 wt %, 0.1 wt % to 3 wt %, 0.1 wt % to 2 wt %, 0.1 wt % to 1 wt %, 0.5 wt % to 5 wt %, 0.5 wt % to 4 wt %, 0.5 wt % to 3 wt %, 0.5 wt % to 2 wt %, or 0.5 wt % to 1 wt %.
Embodiment II-23: The hydrogel composition of any one of Embodiments II-1 to II-18, wherein the concentration of the first agent is 0.5 wt % to 5 wt % and the concentration of the second agent is 0.01 wt % to 4 wt %, the concentration of the first agent is 0.1 wt % to 4 wt % and the concentration of the second agent is 0.01 wt % to 3 wt %, or the concentration of the first agent is 0.5 wt % to 3 wt % and the concentration of the second agent is 0.2 wt % to 2 wt %.
Embodiment II-24: The hydrogel composition of any one of Embodiments II-1 to II-18, wherein the concentration of the first agent is 1 wt % to 3 wt %, or 1 wt % to 2 wt %; and the concentration of the second agent is 0.1 wt % to 2 wt %, 0.1 wt % to 1 wt %, 0.1 wt % to 0.75 wt %, or 0.2 wt % to 0.75 wt %.
Embodiment II-25: The hydrogel composition of any one of Embodiments II-1 to II-18, wherein the concentration of the first agent is about 1.8 wt %, and the concentration of the second agent is about 0.5 wt %.
Embodiment II-26: The hydrogel composition of any one of Embodiments II-1 to II-25, wherein a ratio of the concentration of the first agent to the concentration of the second agent is greater than or equal to 1:1, 1.5:1, 2:1, 3:1, 4:1, 5:1, 10:1, 20:1, or 30:1.
Embodiment II-27: The hydrogel composition of any one of Embodiments II-1 to II-26, wherein a ratio of the concentration of the first agent to the concentration of the second agent is less than or equal to 30:1, 20:1, 10:1, 5:1, 4:1, 3:1, 2:1, 1.5:1, or 1:1.
Embodiment II-28: The hydrogel composition of any one of Embodiments II-1 to II-27, wherein a ratio of the concentration of the first agent to the concentration of the second agent is 1:1 to 10:1, 1.5:1 to 5:1, or 2:1 to 4:1.
Embodiment II-29: The hydrogel composition of any one of Embodiments II-1 to II-28, wherein the hydrogel composition comprises a therapeutic agent.
Embodiment II-30: The hydrogel composition of Embodiment II-29, wherein the therapeutic agent comprises one or more of a small molecule drug, a peptide, a protein, a nucleic acid, a polysaccharide, or cells.
Embodiment II-31: The hydrogel composition of Embodiment II-29, wherein the therapeutic agent is an incretin mimetic.
Embodiment II-32: The hydrogel composition of Embodiment II-29, wherein the therapeutic agent is a glucagon-like peptide-1 receptor agonist (GLP-1 RA).
Embodiment II-33: The hydrogel composition of Embodiment II-31 or II-32, wherein the therapeutic agent is selected from the group consisting of liraglutide, semaglutide, ecnoglutide, cotadutide, mazdutide, tirzepatide, and retatrutide.
Embodiment II-34: The hydrogel composition of Embodiment II-29, wherein the therapeutic agent is a subunit vaccine or a nucleic acid vaccine.
Embodiment II-35: The hydrogel composition of Embodiment II-29, wherein the therapeutic agent is a nucleic acid vaccine comprising at least one nucleic acid molecule encoding one or more antigens for an infectious disease.
Embodiment II-36: The hydrogel composition of Embodiment II-35, wherein the infectious disease is selected from the group consisting of anthrax, botulism, chickenpox, chikungunya fever, cholera, COVID-19, dengue fever, diarrhea, diphtheria, Ebola, encephalitis, gastritis, gastric ulcer, giardiasis, hepatitis, herpes, Hendra virus disease, HIV/AIDS, HPV infection, influenza, listeriosis, Marburg virus disease, malaria, measles, meningitis, meningococcal disease, MERS, mumps, Nipah virus disease, pneumococcal disease, pneumonia, polio, rabies, Ross River fever, RSV infection, rubella, SARS, smallpox, shigellosis, shingles, sleeping sickness, streptococcal disease, tetanus, toxoplasmosis, tuberculosis, typhoid fever, West Nile fever, whooping cough, yellow fever, and yersiniosis.
Embodiment II-37: The hydrogel composition of Embodiment II-35, wherein the one or more antigens comprise an antigen for varicella-zoster virus.
Embodiment II-38: The hydrogel composition of Embodiment II-35, wherein the infectious disease is shingles.
Embodiment II-39: The hydrogel composition of Embodiment II-29, wherein the therapeutic agent is a nucleic acid vaccine comprising at least one nucleic acid molecule encoding one or more antigens that are expressed by tumor cells of a cancer.
Embodiment II-40: The hydrogel composition of Embodiment II-39, wherein the cancer is biliary tract cancer, bladder cancer, brain cancer, breast cancer, cervical cancer, choriocarcinoma, colorectal cancer, endometrial cancer, esophageal cancer, gastric cancer, leukemia, liver cancer, lymphoma, lung cancer, neuroblastoma, oral cancer, ovarian cancer, pancreatic cancer, prostate cancer, renal cancer, sarcoma, skin cancer, testicular cancer, or thyroid cancer.
Embodiment II-41: The hydrogel composition of Embodiment II-29, wherein the therapeutic agent is a nucleic acid vaccine comprising at least one nucleic acid molecule encoding one or more autoantigens.
Embodiment II-42: The hydrogel composition of Embodiment II-41, wherein the one or more autoantigens comprise one or more autoantigens for arthritis, diabetes, inflammatory bowel disease, multiple sclerosis, myasthenia gravis, pemphigus, psoriasis, system lupus erythematosus, or transplant rejection.
Embodiment II-43: The hydrogel composition of any one of Embodiments II-29 or II-34 to II-42, wherein the therapeutic agent is a nucleic acid vaccine comprising a nucleic acid and a delivery vector.
Embodiment II-44: The hydrogel composition of any one of Embodiments II-29 or II-34 to II-42, wherein the therapeutic agent is a nucleic acid vaccine comprising a nucleic acid and a viral vector.
Embodiment II-45: The hydrogel composition of Embodiment II-29, wherein the therapeutic agent is a subunit vaccine comprising an antigen that elicits an immune response in an individual against an infectious disease.
Embodiment II-46: The hydrogel composition of Embodiment II-45, wherein the antigen comprises varicella zoster virus glycoprotein E (gE) antigen.
Embodiment II-47: The hydrogel composition of Embodiment II-45, wherein the infectious disease is selected from the group consisting of anthrax, botulism, chickenpox, chikungunya fever, cholera, COVID-19, dengue fever, diarrhea, diphtheria, Ebola, encephalitis, gastritis, gastric ulcer, giardiasis, hepatitis, herpes, Hendra virus disease, HIV/AIDS, HPV infection, influenza, listeriosis, Marburg virus disease, malaria, measles, meningitis, meningococcal disease, MERS, mumps, Nipah virus disease, pneumococcal disease, pneumonia, polio, rabies, Ross River fever, RSV infection, rubella, SARS, smallpox, shigellosis, sleeping sickness, streptococcal disease, tetanus, toxoplasmosis, tuberculosis, typhoid fever, West Nile fever, whooping cough, yellow fever, and yersiniosis.
Embodiment II-48: The hydrogel composition of any one of Embodiments II-29 or II-34 to II-47, further comprising an adjuvant.
Embodiment II-49: The hydrogel composition of Embodiment II-48, wherein the adjuvant is selected from the group consisting of Monophosphoryl lipid A (MPL), TLR-7/8 agonist 3M-052, cgamp, aluminum, AS01B, AS04, CpG 1018, Matrix-M, and MF59.
Embodiment II-50: The hydrogel composition of any one of Embodiments II-29 to II-46, wherein the hydrogel composition comprises the therapeutic agent in an amount of 0.1 μg to 200 μg, 0.25 μg to 1 μg, 1 μg to 10 μg, 10 μg to 50 μg, 50 μg to 150 μg, or 100 μg to 200 g.
Embodiment II-51: The hydrogel composition of any one of Embodiments II-29 to II-33, wherein the therapeutic agent is an incretin mimetic in an amount of 1 mg to 250 mg.
Embodiment II-52: The hydrogel composition of any one of Embodiments II-1 to II-51, wherein the hydrogel composition has a storage modulus within a range from 1 Pa to 500 Pa, 1 Pa to 250 Pa, 1 Pa to 100 Pa, 1 Pa to 50 Pa, 1 Pa to 25 Pa, 1 Pa to 10 Pa, 10 Pa to 500 Pa, 10 Pa to 250 Pa, 10 Pa to 100 Pa, 10 Pa to 50 Pa, 10 Pa to 25 Pa, 25 Pa to 500 Pa, 25 Pa to 250 Pa, 25 Pa to 100 Pa, 25 Pa to 50 Pa, 50 Pa to 500 Pa, 50 Pa to 250 Pa, 50 Pa to 100 Pa, 100 Pa to 500 Pa, 100 Pa to 250 Pa, or 250 Pa to 500 Pa when measured at 25° C. over an angular frequency of 0.1 rad·s⁻¹ to 100 rad·s⁻¹ within a linear viscoelastic region of the hydrogel composition.

Embodiment II-53: The hydrogel composition of any one of Embodiments II-1 to II-51, wherein the hydrogel composition has a storage modulus within a range from 10 Pa to 1000 Pa, or 50 Pa to 500 Pa when measured at 25° C. over an angular frequency of 0.1 rad·s⁻¹ 1 to 100 rad·s⁻¹ within a linear viscoelastic region of the hydrogel composition.
Embodiment II-54: The hydrogel composition of any one of Embodiments II-1 to II-53, wherein the hydrogel composition has a yield stress within a range from 0.1 Pato 100 Pa, 1 Pa to 50 Pa, 1 Pa to 25 Pa, 1 Pa to 10 Pa, 1 Pa to 5 Pa, 5 Pa to 100 Pa, 5 Pa to 50 Pa, 5 Pa to 25 Pa, 5 Pa to 10 Pa, 10 Pa to 100 Pa, 10 Pa to 50 Pa, 10 Pa to 25 Pa, 25 Pa to 100 Pa, 25 Pa to 50 Pa, or 50 Pa to 100 Pa when measured at 25° C.
Embodiment II-55: The hydrogel composition of any one of Embodiments II-1 to II-53, wherein the hydrogel composition has a yield stress within a range from 1 Pa to 500 Pa, or 20 Pa to 200 Pa when measured at 25° C.
Embodiment II-56: The hydrogel composition of any one of Embodiments II-1 to II-51, wherein the hydrogel composition has a storage modulus of 50 Pa to 500 Pa when measured at 25° C. over an angular frequency of 0.1 rad·s′″ to 100 rad·s⁻¹ within a linear viscoelastic region of the hydrogel composition; and/or a yield stress of 20 Pa to 200 Pa when measured at 25° C. at an angular frequency of 10 rad·s⁻¹ within a linear viscoelastic region of the hydrogel composition.
Embodiment II-57: The hydrogel composition of any one of Embodiments II-1 to II-51, wherein the hydrogel composition has a storage modulus of 100 Pa to 200 Pa when measured at 25° C. over an angular frequency of 0.1 rad·s⁻¹ to 100 rad·s⁻¹ within a linear viscoelastic region of the hydrogel composition; and/or a yield stress of 50 Pa to 1000 Pa when measured at 25° C. at an angular frequency of 10 rad·s⁻¹ within a linear viscoelastic region of the hydrogel composition.
Embodiment II-58: The hydrogel composition of any one of Embodiments II-1 to II-57, wherein the hydrogel composition has a viscosity less than 10,000 mPa-s when measured at 25° C. at a shear rate of 1000 s⁻¹.
Embodiment II-59: The hydrogel composition of any one of Embodiments II-1 to II-57, wherein the hydrogel composition has a viscosity within a range from 100 mPa-s to 1000 mPa-s when measured at 25° C. at a shear rate of 1000 s⁻¹.
Embodiment II-60: The hydrogel composition of any one of Embodiments II-1 to II-59, wherein the hydrogel composition has an extensional strain to break of at least 500%, 1000%, 1500%, or 2000% when measured at 25° C. at a strain rate from 0.06 s⁻¹ to 0.3 s⁻¹.
Embodiment II-61: The hydrogel composition of any one of Embodiments II-1 to II-60, wherein the hydrogel composition has a crossover frequency greater than or equal to 0.001 rad·s⁻¹, 0.01 rad·s⁻¹, or 0.1 rad·s⁻¹ when measured at 25° C. within a linear viscoelastic region of the hydrogel composition.
Embodiment II-62: The hydrogel composition of any one of Embodiments II-1 to II-61, wherein the hydrogel composition has a crossover frequency that is a greater than a crossover frequency of a corresponding hydrogel composition comprising the first agent without the second agent.
Embodiment II-63: The hydrogel composition of any one of Embodiments II-1 to II-62, wherein the hydrogel composition comprises a therapeutic agent and upon administration to an individual, the therapeutic agent is released from the hydrogel composition into a surrounding injection site over a period of at least 1 day, 2 days, 3 days, 4 days, 5 days, 6 days, 7 days, 8 days, 9 days, 10 days, 12 days, or 14 days.
Embodiment II-64: The hydrogel composition of any one of Embodiments II-1 to II-62, wherein the hydrogel composition comprises a therapeutic agent and upon administration to an individual, the therapeutic agent is released from the hydrogel composition into a surrounding injection site over a period of less than or equal to 14 days, 12 days, 10 days, 8 days, 7 days, 6 days, 5 days, 4 days, 3 days, 2 days, or 1 day.
Embodiment II-65: The hydrogel composition of any one of Embodiments II-1 to II-64, wherein the hydrogel composition comprises a therapeutic agent and upon administration of the hydrogel composition to an individual in need thereof, the therapeutic agent is released from the hydrogel composition to the individual at a rate of 0.25 mg/week to 25 mg/week, 0.5 mg/week to 20 mg/week, 0.5 mg/week to 2 mg/week, 0.5 mg/week to 1.5 mg/week, 1 mg/week to 2 mg/week, 2 mg/week to 5 mg/week, 5 mg/week to 15 mg/week, 5 mg/week to 10 mg/week, 10 mg/week to 20 mg/week, 10 mg/week to 15 mg/week, 12 mg/week to 25 mg/week, 12 mg/week to 15 mg/week, 15 mg/week to 25 mg/week, 15 mg/week to 20 mg/week, or 20 mg/week to 25 mg/week.
Embodiment II-66: The hydrogel composition of any one of Embodiments II-1 to II-65, wherein the hydrogel composition persists at an administration site for a duration of time of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 21 days, at least 28 days, at least 1 month, at least 2 months, at least 3 months, at least 4 months, at least 5 months, at least 6 months, at least 9 months, or at least 12 months.
Embodiment II-67: The hydrogel composition of any one of Embodiments II-1 to II-65, wherein the hydrogel composition persists at an administration site for a duration of time for no more than 12 months, no more than 9 months, no more than 6 months, no more than 5 months, no more than 4 months, no more than 3 months, no more than 2 months, no more than 1 month, no more than 28 days, no more than 21 days, no more than 14 days, no more than 13 days, no more than 12 days, no more than 11 days, no more than 10 days, no more than 9 days, no more than 8 days, no more than 7 days, no more than 6 days, no more than 5 days, no more than 4 days, no more than 3 days, no more than 2 days, or no more than 1 day.
Embodiment II-68: The hydrogel composition of any one of Embodiments II-1 to II-65, wherein the hydrogel composition persists at an administration site for a duration of time of 1 day to 30 days, 1 day to 14 days, 1 day to 7 days, 1 day to 3 days, 3 days to 30 days, 3 days to 14 days, 3 days to 7 days, 7 days to 30 days, 7 days to 14 days, or 14 days to 30 days.
Embodiment II-69: The hydrogel composition of any one of Embodiments II-66 to II-68, wherein the amount of hydrogel composition that persists at the administration site for the duration of time is at least 95%, at least 90%, at least 85%, at least 80%, at least 75%, at least 70%, at least 60%, at least 50%, at least 40%, or at least 30% of an initial hydrogel composition amount administered to an individual in need thereof.
Embodiment II-70: The hydrogel composition of any one of Embodiments II-1 to II-69, wherein, upon administration to an individual, the hydrogel composition forms a depot that permits infiltration by a target cell and inhibits infiltration by a nontarget cell.
Embodiment II-71: The hydrogel composition of Embodiment II-70, wherein the target cell is an immune cell or an antigen-presenting cell, and the nontarget cell is a nonimmune cell.
Embodiment II-72: The hydrogel composition of any one of Embodiments II-1 to II-71, wherein the hydrogel composition is configured for administration via injection.
Embodiment II-73: The hydrogel composition of any one of Embodiments II-1 to II-71, wherein the hydrogel composition is configured for 3D printing.
Embodiment II-74: The hydrogel composition of Embodiment II-73, wherein the hydrogel composition is configured to be extruded from a nozzle as a continuous filament.
Embodiment II-75: The hydrogel composition of Embodiment II-73 or II-74, wherein the hydrogel composition has an extensional strain to break of at least 500%, 1000%, 1500%, or 2000% when measured at 25° C. at a strain rate from 0.06 s⁻¹ to 0.3 s⁻¹.
Embodiment II-76: The hydrogel composition of any one of Embodiments II-73 to II-75, further comprising one or more reactive species that polymerize upon exposure to energy to form a crosslinked polymer network.
Embodiment II-77: The hydrogel composition of Embodiment II-76, wherein the one or more reactive species comprise one or more of a monomer, an oligomer, a reactive polymer, a photoinitiator, or a thermal initiator.
Embodiment II-78: The hydrogel composition of any one of claims 1 to 28, 52 to 62, or 73 to 77, wherein the hydrogel composition does not comprise a therapeutic agent.
Embodiment II-79: A method of treating or preventing a disease or condition in an individual in need thereof, comprising administering to an individual in need thereof the hydrogel composition according to any one of Embodiments II-1 to II-78.
Embodiment II-80: A composition for use in treating or preventing a disease or condition in an individual in need thereof, wherein the composition for use is a hydrogel composition according to any one of Embodiments II-1 to II-78.
Embodiment II-81: The method of Embodiment II-79, or composition for use of Embodiment II-80, wherein the disease or condition is an infectious disease, cancer, an autoimmune disease or condition, diabetes, a diabetes-related condition, or tissue adhesion.
Embodiment II-82: The method of Embodiment II-79, or composition for use of Embodiment II-80, wherein the disease or condition is diabetes or a diabetes-related condition.
Embodiment II-83: The method or composition for use of Embodiment II-82, wherein the diabetes or diabetes-related condition is selected from the group consisting of prediabetes, type 1 diabetes, type 2 diabetes, hyperglycemia, hypoglycemia, and impaired glucose tolerance.
Embodiment II-84: The method or composition for use of Embodiment II-82, wherein the method or compound for use is to treat or prevent hyperglycemia.
Embodiment II-85: The method or composition for use of Embodiment II-82, wherein the method or compound for use is to treat or prevent hypoglycemia.
Embodiment II-86: The method of Embodiment II-79, or composition for use of Embodiment II-80, wherein the disease or condition is an infectious disease selected from the group consisting of anthrax, botulism, chickenpox, chikungunya fever, cholera, COVID-19, dengue fever, diarrhea, diphtheria, Ebola, encephalitis, gastritis, gastric ulcer, giardiasis, hepatitis, herpes, Hendra virus disease, HIV/AIDS, HPV infection, influenza, listeriosis, Marburg virus disease, malaria, measles, meningitis, meningococcal disease, MERS, mumps, Nipah virus disease, pneumococcal disease, pneumonia, polio, rabies, Ross River fever, RSV infection, rubella, SARS, smallpox, shigellosis, shingles, sleeping sickness, streptococcal disease, tetanus, toxoplasmosis, tuberculosis, typhoid fever, West Nile fever, whooping cough, yellow fever, and yersiniosis.
Embodiment II-87: The method or composition for use of Embodiment II-86, wherein the infectious disease is shingles.
Embodiment II-88: The method of Embodiment II-79, or composition for use of Embodiment II-80, wherein the disease or condition is an autoimmune disease or condition selected from the group consisting of arthritis, diabetes, inflammatory bowel disease, multiple sclerosis, myasthenia gravis, pemphigus, psoriasis, system lupus erythematosus, and transplant rejection.
Embodiment II-89: The method of Embodiment II-79, or composition for use of Embodiment II-80, wherein the disease or condition is cancer.
Embodiment II-90: The method or use of Embodiment II-89, wherein the cancer is biliary tract cancer, bladder cancer, brain cancer, breast cancer, cervical cancer, choriocarcinoma, colorectal cancer, endometrial cancer, esophageal cancer, gastric cancer, leukemia, liver cancer, lymphoma, lung cancer, neuroblastoma, oral cancer, ovarian cancer, pancreatic cancer, prostate cancer, renal cancer, sarcoma, skin cancer, testicular cancer, or thyroid cancer.
Embodiment II-91: The method of any one of Embodiments II-79 or II-81 to II-90, or composition for use of any one of Embodiments II-80 to II-90, wherein the hydrogel composition is administered by injection to an individual in need thereof, and the injection is subcutaneous or intramuscular.
Embodiment II-92: The method of any one of Embodiments II-79 or II-81 to II-91, or composition for use of any one of Embodiments II-80 to II-91, wherein after administration of the hydrogel composition to the individual in need thereof, the blood serum of the individual has a therapeutic agent concentration of 50 ng/ml to 500 ng/ml, 100 ng/ml to 300 ng/mL, or 150 ng/mL to 250 ng/mL over a period of 1 day to 30 days, 1 day to 14 days, 1 day to 7 days, or 1 day to 3 days.
Embodiment II-93: The method of any one of Embodiments II-79 or II-81 to II-91, or composition for use of any one of Embodiments II-80 to II-91, wherein after administration of the hydrogel composition to the individual in need thereof, the blood serum of the individual has a therapeutic agent concentration of 50 ng/ml to 500 ng/mL, 100 ng/ml to 300 ng/mL, or 150 ng/mL to 250 ng/mL for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 21 days, at least 28 days, at least 1 month, at least 2 months, at least 3 months, at least 4 months, at least 5 months, at least 6 months, at least 9 months, or at least 12 months.
Embodiment II-94: The method of any one of Embodiments II-79 or II-81 to II-91, or composition for use of any one of Embodiments II-80 to II-91, wherein after administration of the hydrogel composition to the individual in need thereof, the blood serum of the individual has a therapeutic agent concentration of 50 ng/ml to 500 ng/ml, for at least 7 days or at least 14 days.
Embodiment II-95: The method of Embodiment II-79, or composition for use of Embodiment II-80, wherein the method and use is for treating or preventing tissue adhesion in an individual in need thereof.
Embodiment II-96: The method or composition for use of Embodiment II-95, wherein the hydrogel composition does not comprise a therapeutic agent.
Embodiment II-97: The method or composition for use of Embodiment II-95, wherein the hydrogel composition comprises a therapeutic agent.
Embodiment II-98: The method or composition for use of any one of Embodiments II-95 to II-97, wherein the method or use comprises forming an incision in tissue of the individual in need thereof; and applying a hydrogel to tissue through the incision.
Embodiment II-99: The method or composition for use of Embodiment II-98, wherein the forming the incision in the tissue is part of a surgical procedure.
Embodiment II-100: The method or composition for use of any one of Embodiments II-95 to II-99, wherein the hydrogel composition remains on the tissue for at least 7 days or at least 14 days after applying to the individual in need thereof.
Embodiment II-101: The method or composition for use of any one of Embodiments II-95 to II-100, wherein the hydrogel composition dissipates from the tissue in less than 120 days or less than 30 days after applying to the individual in need thereof.
Embodiment II-102: The method or composition for use of any one of Embodiments II-95 to II-101, wherein applying comprises spraying, spreading, or injecting the hydrogel composition onto the tissue.
Embodiment II-103: The method or composition for use of any one of Embodiments II-95 to II-102, wherein the tissue comprises abdominal, orthopedic, thoracic, gynecologic, or cardiac tissue.
Embodiment II-104: A method for delivering a therapeutic agent to an individual in need thereof, comprising injecting the hydrogel composition according to any one of Embodiments II-1 to II-78 into the individual in need thereof.
Embodiment II-105: A hydrogel composition according to any one of Embodiments II-1 to II-78 for use in 3D printing.
Embodiment II-106: A method for forming an object, comprising depositing a hydrogel composition according to any one of Embodiments II-1 to II-78 on a surface using a 3D printing process.
Embodiment II-107: The method of Embodiment II-106, wherein the 3D printing process comprises an extrusion-based process.
Embodiment II-108: The method of Embodiment II-107, wherein the extrusion-based process comprises extruding the hydrogel composition from a nozzle to form a filament.
Embodiment II-109: The method of Embodiment II-108, wherein the filament has a diameter that is the same as or smaller than a diameter of the nozzle.
Embodiment II-110: The method of Embodiment II-109, wherein the diameter of the filament is no more than 2 mm, 1 mm, 900 μm, 800 μm, 700 μm, 600 μm, 500 μm, 400 μm, 300 μm, 200 μm, 100 μm, 50 μm, 20 μm, or 10 μm.
Embodiment II-111: The method of Embodiment II-109 or II-110, wherein the diameter of the nozzle is at least 100 μm, 200 μm, 300 μm, 400 μm, 500 μm, 600 μm, 700 μm, 800 μm, 900 μm, 1 mm, or 2 mm.
Embodiment II-112: The method of any one of Embodiments II-108 to II-111, further comprising stretching the filament after extruding the filament from the nozzle to reduce the diameter of the filament.
Embodiment II-113: The method of any one of Embodiments II-106 to II-112, wherein the hydrogel composition comprises one or more reactive species, and the method further comprises applying energy to the hydrogel composition to cause the one or more reactive species to form a crosslinked polymer network.
Embodiment II-114: The method of any one of Embodiments II-106 to II-113, wherein the object is a tissue engineering scaffold, a drug delivery depot, or at least a portion of a medical device.

I/We claim:

1. A hydrogel composition, comprising:
a first agent, wherein the first agent is octadecyl modified hydroxypropyl methylcellulose (HPMC-C18), and wherein the first agent forms micellar structures; and
a second agent comprising a surfactant or cyclic polysaccharide, wherein the second agent disrupts the micellar structures of the first agent.

2. The hydrogel composition of claim 1, wherein the second agent is a non-ionic surfactant.

3. The hydrogel composition of claim 2, wherein the non-ionic surfactant has a hydrophilic-lipophilic balance (HLB) value of at least 9, at least 10, at least 11, at least 12, at least 13, at least 14, or at least 15.

4. The hydrogel composition of claim 2 or 3, wherein the non-ionic surfactant is a polysorbate or a polyoxyethylene fatty ether.

5. The hydrogel composition of claim 4, wherein the polysorbate is selected from the group consisting of polyoxyethylene (20) sorbitan monooleate (polysorbate 80), polyoxyethylene (20) sorbitan monolaurate (polysorbate 20), polyoxyethylene (20) sorbitan monopalmitate (polysorbate 40), and polyoxyethylene (20) sorbitan monostearate (polysorbate 60).

6. The hydrogel composition of any one of claims 1 to 5, wherein the second agent is polyoxyethylene (20) sorbitan monolaurate (polysorbate 20).

7. The hydrogel composition of any one of claims 1 to 6, wherein the second agent is polyoxyethylene (20) sorbitan monooleate (polysorbate 80).

8. The hydrogel composition of claim 4, wherein the polyoxyethylene fatty ether comprises a hydrocarbon chain of 12 to 18 carbons and 2 to 100 oxyethylene groups.

9. The hydrogel composition of claim 1, wherein the second agent is a cyclic polysaccharide.

10. The hydrogel composition of claim 9, wherein the second agent is a hexasaccharide.

11. The hydrogel composition of claim 9, wherein the second agent is α-cyclodextrin.

12. The hydrogel composition of any one of claims 1 to 11, wherein the micellar structures comprise C18 side chains of the HPMC-C18, and wherein the second agent is configured to noncovalently interact with the C18 side chains.

13. The hydrogel composition of claim 12 wherein the second agent comprises a hydrophobic tail having a length different than a length of the C18 side chains of the HPMC-C18.

14. The hydrogel composition of claim 12 or 13, wherein the second agent comprises a hydrophobic tail having an unsaturated hydrocarbon group.

15. The hydrogel composition of any one of claims 12 to 14, wherein the second agent is configured to disrupt packing and/or organized assembly of the C18 side chains within the micellar structures.

16. The hydrogel composition of any one of claims 12 to 15, wherein the second agent is configured to create free volume between the C18 side chains within the micellar structures.

17. The hydrogel composition of any one of claims 12 to 16, wherein the second agent is dispersed within the micellar structures to form at least one mixed micelle.

18. The hydrogel composition of any one of claims 12 to 17, wherein the second agent binds to and prevents at least some of the C18 side chains from entering the micellar structures.

19. The hydrogel composition of any one of claims 1 to 18, wherein the concentration of the first agent is at least 0.1 wt %, at least 0.25 wt %, at least 0.5 wt %, at least 0.75 wt %, at least 1 wt %, at least 1.5 wt %, at least 2 wt %, at least 2.5 wt %, at least 3 wt %, at least 3.5 wt %, at least 4 wt %, at least 4.5 wt %, or at least 5 wt %.

20. The hydrogel composition of any one of claims 1 to 18, wherein the concentration of the first agent is 0.1 wt % to 5 wt %, 0.1 wt % to 4 wt %, 0.1 wt % to 3 wt %, 0.1 wt % to 2 wt %, 0.1 wt % to 1 wt %, 0.1 wt % to 0.5 wt %, 0.5 wt % to 5 wt %, 0.5 wt % to 4 wt %, 0.5 wt % to 3 wt %, 0.5 wt % to 2 wt %, 0.5 wt % to 1 wt %, 1 wt % to 5 wt %, 1 wt % to 4 wt %, 1 wt % to 3 wt %, 1 wt % to 2 wt %, 2 wt % to 5 wt %, 2 wt % to 4 wt %, 2 wt % to 3 wt %, 3 wt % to 5 wt %, 3 wt % to 4 wt %, or 4 wt % to 5 wt %.

21. The hydrogel composition of any one of claims 1 to 20, wherein the concentration of the second agent is at least 0.02 wt %, at least 0.1 wt %, at least 0.5 wt %, at least 1 wt %, at least 2 wt %, at least 3 wt %, at least 4 wt %, or at least 5 wt %.

22. The hydrogel composition of any one of claims 1 to 20, wherein the concentration of the second agent is 0.02 wt % to 5 wt %, 0.02 wt % to 4 wt %, 0.02 wt % to 3 wt %, 0.02 wt % to 2 wt %, 0.02 wt % to 1 wt %, 0.1 wt % to 5 wt %, 0.1 wt % to 4 wt %, 0.1 wt % to 3 wt %, 0.1 wt % to 2 wt %, 0.1 wt % to 1 wt %, 0.5 wt % to 5 wt %, 0.5 wt % to 4 wt %, 0.5 wt % to 3 wt %, 0.5 wt % to 2 wt %, or 0.5 wt % to 1 wt %.

23. The hydrogel composition of any one of claims 1 to 18, wherein the concentration of the first agent is 0.5 wt % to 5 wt % and the concentration of the second agent is 0.01 wt % to 4 wt %, the concentration of the first agent is 0.1 wt % to 4 wt % and the concentration of the second agent is 0.01 wt % to 3 wt %, or the concentration of the first agent is 0.5 wt % to 3 wt % and the concentration of the second agent is 0.2 wt % to 2 wt %.

24. The hydrogel composition of any one of claims 1 to 18, wherein the concentration of the first agent is 1 wt % to 3 wt %, or 1 wt % to 2 wt %; and the concentration of the second agent is 0.1 wt % to 2 wt %, 0.1 wt % to 1 wt %, 0.1 wt % to 0.75 wt %, or 0.2 wt % to 0.75 wt %.

25. The hydrogel composition of any one of claims 1 to 18, wherein the concentration of the first agent is about 1.8 wt %, and the concentration of the second agent is about 0.5 wt %.

26. The hydrogel composition of any one of claims 1 to 25, wherein a ratio of the concentration of the first agent to the concentration of the second agent is greater than or equal to 1:1, 1.5:1, 2:1, 3:1, 4:1, 5:1, 10:1, 20:1, or 30:1.

27. The hydrogel composition of any one of claims 1 to 26, wherein a ratio of the concentration of the first agent to the concentration of the second agent is less than or equal to 30:1, 20:1, 10:1, 5:1, 4:1, 3:1, 2:1, 1.5:1, or 1:1.

28. The hydrogel composition of any one of claims 1 to 27, wherein a ratio of the concentration of the first agent to the concentration of the second agent is 1:1 to 10:1, 1.5:1 to 5:1, or 2:1 to 4:1.

29. The hydrogel composition of any one of claims 1 to 28, wherein the hydrogel composition comprises a therapeutic agent.

30. The hydrogel composition of claim 29, wherein the therapeutic agent comprises one or more of a small molecule drug, a peptide, a protein, a nucleic acid, a polysaccharide, or cells.

31. The hydrogel composition of claim 29, wherein the therapeutic agent is an incretin mimetic.

32. The hydrogel composition of claim 29, wherein the therapeutic agent is a glucagon-like peptide-1 receptor agonist (GLP-1 RA).

33. The hydrogel composition of claim 31 or 32, wherein the therapeutic agent is selected from the group consisting of liraglutide, semaglutide, ecnoglutide, cotadutide, mazdutide, tirzepatide, and retatrutide.

34. The hydrogel composition of claim 29, wherein the therapeutic agent is a subunit vaccine or a nucleic acid vaccine.

35. The hydrogel composition of claim 29, wherein the therapeutic agent is a nucleic acid vaccine comprising at least one nucleic acid molecule encoding one or more antigens for an infectious disease.

36. The hydrogel composition of claim 35, wherein the infectious disease is selected from the group consisting of anthrax, botulism, chickenpox, chikungunya fever, cholera, COVID-19, dengue fever, diarrhea, diphtheria, Ebola, encephalitis, gastritis, gastric ulcer, giardiasis, hepatitis, herpes, Hendra virus disease, HIV/AIDS, HPV infection, influenza, listeriosis, Marburg virus disease, malaria, measles, meningitis, meningococcal disease, MERS, mumps, Nipah virus disease, pneumococcal disease, pneumonia, polio, rabies, Ross River fever, RSV infection, rubella, SARS, smallpox, shigellosis, shingles, sleeping sickness, streptococcal disease, tetanus, toxoplasmosis, tuberculosis, typhoid fever, West Nile fever, whooping cough, yellow fever, and yersiniosis.

37. The hydrogel composition of claim 35, wherein the one or more antigens comprise an antigen for varicella-zoster virus.

38. The hydrogel composition of claim 35, wherein the infectious disease is shingles.

39. The hydrogel composition of claim 29, wherein the therapeutic agent is a nucleic acid vaccine comprising at least one nucleic acid molecule encoding one or more antigens that are expressed by tumor cells of a cancer.

40. The hydrogel composition of claim 39, wherein the cancer is biliary tract cancer, bladder cancer, brain cancer, breast cancer, cervical cancer, choriocarcinoma, colorectal cancer, endometrial cancer, esophageal cancer, gastric cancer, leukemia, liver cancer, lymphoma, lung cancer, neuroblastoma, oral cancer, ovarian cancer, pancreatic cancer, prostate cancer, renal cancer, sarcoma, skin cancer, testicular cancer, or thyroid cancer.

41. The hydrogel composition of claim 29, wherein the therapeutic agent is a nucleic acid vaccine comprising at least one nucleic acid molecule encoding one or more autoantigens.

42. The hydrogel composition of claim 41, wherein the one or more autoantigens comprise one or more autoantigens for arthritis, diabetes, inflammatory bowel disease, multiple sclerosis, myasthenia gravis, pemphigus, psoriasis, system lupus erythematosus, or transplant rejection.

43. The hydrogel composition of any one of claims 29 or 34 to 42, wherein the therapeutic agent is a nucleic acid vaccine comprising a nucleic acid and a delivery vector.

44. The hydrogel composition of any one of claims 29 or 34 to 42, wherein the therapeutic agent is a nucleic acid vaccine comprising a nucleic acid and a viral vector.

45. The hydrogel composition of claim 29, wherein the therapeutic agent is a subunit vaccine comprising an antigen that elicits an immune response in an individual against an infectious disease.

46. The hydrogel composition of claim 45, wherein the antigen comprises varicella zoster virus glycoprotein E (gE) antigen.

47. The hydrogel composition of claim 45, wherein the infectious disease is selected from the group consisting of anthrax, botulism, chickenpox, chikungunya fever, cholera, COVID-19, dengue fever, diarrhea, diphtheria, Ebola, encephalitis, gastritis, gastric ulcer, giardiasis, hepatitis, herpes, Hendra virus disease, HIV/AIDS, HPV infection, influenza, listeriosis, Marburg virus disease, malaria, measles, meningitis, meningococcal disease, MERS, mumps, Nipab virus disease, pneumococcal disease, pneumonia, polio, rabies, Ross River fever, RSV infection, rubella, SARS, smallpox, shigellosis, sleeping sickness, streptococcal disease, tetanus, toxoplasmosis, tuberculosis, typhoid fever, West Nile fever, whooping cough, yellow fever, and yersiniosis.

48. The hydrogel composition of any one of claims 29 or 34 to 47, further comprising an adjuvant.

49. The hydrogel composition of claim 48, wherein the adjuvant is selected from the group consisting of Monophosphoryl lipid A (MPL), TLR-7/8 agonist 3M-052, cgamp, aluminum, AS01B, AS04, CpG 1018, Matrix-M, and MF59.

50. The hydrogel composition of any one of claims 29 to 46, wherein the hydrogel composition comprises the therapeutic agent in an amount of 0.1 μg to 200 μg, 0.25 μg to 1 μg, 1 μg to 10 μg, 10 μg to 50 μg, 50 μg to 150 μg, or 100 μg to 200 μg.

51. The hydrogel composition of any one of claims 29 to 33, wherein the therapeutic agent is an incretin mimetic in an amount of 1 mg to 250 mg.

52. The hydrogel composition of any one of claims 1 to 51, wherein the hydrogel composition has a storage modulus within a range from 1 Pa to 500 Pa, 1 Pa to 250 Pa, 1 Pa to 100 Pa, 1 Pa to 50 Pa, 1 Pa to 25 Pa, 1 Pa to 10 Pa, 10 Pa to 500 Pa, 10 Pa to 250 Pa, 10 Pa to 100 Pa, 10 Pa to 50 Pa, 10 Pa to 25 Pa, 25 Pa to 500 Pa, 25 Pa to 250 Pa, 25 Pa to 100 Pa, 25 Pa to 50 Pa, 50 Pa to 500 Pa, 50 Pa to 250 Pa, 50 Pa to 100 Pa, 100 Pa to 500 Pa, 100 Pa to 250 Pa, or 250 Pa to 500 Pa when measured at 25° C. over an angular frequency of 0.1 rad·s⁻¹ to 100 rad·s⁻¹ within a linear viscoelastic region of the hydrogel composition.

53. The hydrogel composition of any one of claims 1 to 51, wherein the hydrogel composition has a storage modulus within a range from 10 Pa to 1000 Pa, or 50 Pa to 500 Pa when measured at 25° C. over an angular frequency of 0.1 rad·s⁻¹ to 100 rad·s⁻¹ within a linear viscoelastic region of the hydrogel composition.

54. The hydrogel composition of any one of claims 1 to 53, wherein the hydrogel composition has a yield stress within a range from 0.1 Pa to 100 Pa, 1 Pa to 50 Pa, 1 Pa to 25 Pa, 1 Pa to 10 Pa, 1 Pa to 5 Pa, 5 Pa to 100 Pa, 5 Pa to 50 Pa, 5 Pa to 25 Pa, 5 Pa to 10 Pa, 10 Pa to 100 Pa, 10 Pa to 50 Pa, 10 Pa to 25 Pa, 25 Pa to 100 Pa, 25 Pa to 50 Pa, or 50 Pa to 100 Pa when measured at 25° C.

55. The hydrogel composition of any one of claims 1 to 53, wherein the hydrogel composition has a yield stress within a range from 1 Pa to 500 Pa, or 20 Pa to 200 Pa when measured at 25° C.

56. The hydrogel composition of any one of claims 1 to 51, wherein the hydrogel composition has a storage modulus of 50 Pa to 500 Pa when measured at 25° C. over an angular frequency of 0.1 rad·s⁻¹ to 100 rad·s⁻¹ within a linear viscoelastic region of the hydrogel composition; and/or a yield stress of 20 Pa to 200 Pa when measured at 25° C. at an angular frequency of 10 rad·s⁻¹ within a linear viscoelastic region of the hydrogel composition.

57. The hydrogel composition of any one of claims 1 to 51, wherein the hydrogel composition has a storage modulus of 100 Pa to 200 Pa when measured at 25° C. over an angular frequency of 0.1 rad·s⁻¹ to 100 rad·s⁻¹ within a linear viscoelastic region of the hydrogel composition; and/or a yield stress of 50 Pa to 1000 Pa when measured at 25° C. at an angular frequency of 10 rad·s⁻¹ within a linear viscoelastic region of the hydrogel composition.

58. The hydrogel composition of any one of claims 1 to 57, wherein the hydrogel composition has a viscosity less than 10,000 mPa-s when measured at 25° C. at a shear rate of 1000 s⁻¹.

59. The hydrogel composition of any one of claims 1 to 57, wherein the hydrogel composition has a viscosity within a range from 100 mPa-s to 1000 mPa-s when measured at 25° C. at a shear rate of 1000 s⁻¹.

60. The hydrogel composition of any one of claims 1 to 59, wherein the hydrogel composition has an extensional strain to break of at least 500%, 1000%, 1500%, or 2000% when measured at 25° C. at a strain rate from 0.06 s⁻¹ to 0.3 s⁻¹.

61. The hydrogel composition of any one of claims 1 to 60, wherein the hydrogel composition has a crossover frequency greater than or equal to 0.001 rad·s⁻¹, 0.01 rad·s⁻¹, or 0.1 rad·s⁻¹ when measured at 25° C. within a linear viscoelastic region of the hydrogel composition.

62. The hydrogel composition of any one of claims 1 to 61, wherein the hydrogel composition has a crossover frequency that is a greater than a crossover frequency of a corresponding hydrogel composition comprising the first agent without the second agent.

63. The hydrogel composition of any one of claims 1 to 62, wherein the hydrogel composition comprises a therapeutic agent and upon administration to an individual, the therapeutic agent is released from the hydrogel composition into a surrounding injection site over a period of at least 1 day, 2 days, 3 days, 4 days, 5 days, 6 days, 7 days, 8 days, 9 days, 10 days, 12 days, or 14 days.

64. The hydrogel composition of any one of claims 1 to 62, wherein the hydrogel composition comprises a therapeutic agent and upon administration to an individual, the therapeutic agent is released from the hydrogel composition into a surrounding injection site over a period of less than or equal to 14 days, 12 days, 10 days, 8 days, 7 days, 6 days, 5 days, 4 days, 3 days, 2 days, or 1 day.

65. The hydrogel composition of any one of claims 1 to 64, wherein the hydrogel composition comprises a therapeutic agent and upon administration of the hydrogel composition to an individual in need thereof, the therapeutic agent is released from the hydrogel composition to the individual at a rate of 0.25 mg/week to 25 mg/week, 0.5 mg/week to 20 mg/week, 0.5 mg/week to 2 mg/week, 0.5 mg/week to 1.5 mg/week, 1 mg/week to 2 mg/week, 2 mg/week to 5 mg/week, 5 mg/week to 15 mg/week, 5 mg/week to 10 mg/week, 10 mg/week to 20 mg/week, 10 mg/week to 15 mg/week, 12 mg/week to 25 mg/week, 12 mg/week to 15 mg/week, 15 mg/week to 25 mg/week, 15 mg/week to 20 mg/week, or 20 mg/week to 25 mg/week.

66. The hydrogel composition of any one of claims 1 to 65, wherein the hydrogel composition persists at an administration site for a duration of time of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 21 days, at least 28 days, at least 1 month, at least 2 months, at least 3 months, at least 4 months, at least 5 months, at least 6 months, at least 9 months, or at least 12 months.

67. The hydrogel composition of any one of claims 1 to 65, wherein the hydrogel composition persists at an administration site for a duration of time for no more than 12 months, no more than 9 months, no more than 6 months, no more than 5 months, no more than 4 months, no more than 3 months, no more than 2 months, no more than 1 month, no more than 28 days, no more than 21 days, no more than 14 days, no more than 13 days, no more than 12 days, no more than 11 days, no more than 10 days, no more than 9 days, no more than 8 days, no more than 7 days, no more than 6 days, no more than 5 days, no more than 4 days, no more than 3 days, no more than 2 days, or no more than 1 day.

68. The hydrogel composition of any one of claims 1 to 65, wherein the hydrogel composition persists at an administration site for a duration of time of 1 day to 30 days, 1 day to 14 days, 1 day to 7 days, 1 day to 3 days, 3 days to 30 days, 3 days to 14 days, 3 days to 7 days, 7 days to 30 days, 7 days to 14 days, or 14 days to 30 days.

69. The hydrogel composition of any one of claims 66 to 68, wherein the amount of hydrogel composition that persists at the administration site for the duration of time is at least 95%, at least 90%, at least 85%, at least 80%, at least 75%, at least 70%, at least 60%, at least 50%, at least 40%, or at least 30% of an initial hydrogel composition amount administered to an individual in need thereof.

70. The hydrogel composition of any one of claims 1 to 69, wherein, upon administration to an individual, the hydrogel composition forms a depot that permits infiltration by a target cell and inhibits infiltration by a nontarget cell.

71. The hydrogel composition of claim 70, wherein the target cell is an immune cell or an antigen-presenting cell, and the nontarget cell is a nonimmune cell.

72. The hydrogel composition of any one of claims 1 to 71, wherein the hydrogel composition is configured for administration via injection.

73. The hydrogel composition of any one of claims 1 to 71, wherein the hydrogel composition is configured for 3D printing.

74. The hydrogel composition of claim 73, wherein the hydrogel composition is configured to be extruded from a nozzle as a continuous filament.

75. The hydrogel composition of claim 73 or 74, wherein the hydrogel composition has an extensional strain to break of at least 500%, 1000%, 1500%, or 2000% when measured at 25° C. at a strain rate from 0.06 s⁻¹ to 0.3 s⁻¹.

76. The hydrogel composition of any one of claims 73 to 75, further comprising one or more reactive species that polymerize upon exposure to energy to form a crosslinked polymer network.

77. The hydrogel composition of claim 76, wherein the one or more reactive species comprise one or more of a monomer, an oligomer, a reactive polymer, a photoinitiator, or a thermal initiator.

78. The hydrogel composition of any one of claims 1 to 28, 52 to 62, or 73 to 77, wherein the hydrogel composition does not comprise a therapeutic agent.

79. A method of treating or preventing a disease or condition in an individual in need thereof, comprising administering to an individual in need thereof the hydrogel composition according to any one of claims 1 to 78.

80. A composition for use in treating or preventing a disease or condition in an individual in need thereof, wherein the composition for use is a hydrogel composition according to any one of claims 1 to 78.

81. The method of claim 79, or composition for use of claim 80, wherein the disease or condition is an infectious disease, cancer, an autoimmune disease or condition, diabetes, a diabetes-related condition, or tissue adhesion.

82. The method of claim 79, or composition for use of claim 80, wherein the disease or condition is diabetes or a diabetes-related condition.

83. The method or composition for use of claim 82, wherein the diabetes or diabetes-related condition is selected from the group consisting of prediabetes, type 1 diabetes, type 2 diabetes, hyperglycemia, hypoglycemia, and impaired glucose tolerance.

84. The method or composition for use of claim 82, wherein the method or compound for use is to treat or prevent hyperglycemia.

85. The method or composition for use of claim 82, wherein the method or compound for use is to treat or prevent hypoglycemia.

86. The method of claim 79, or composition for use of claim 80, wherein the disease or condition is an infectious disease selected from the group consisting of anthrax, botulism, chickenpox, chikungunya fever, cholera, COVID-19, dengue fever, diarrhea, diphtheria, Ebola, encephalitis, gastritis, gastric ulcer, giardiasis, hepatitis, herpes, Hendra virus disease, HIV/AIDS, HPV infection, influenza, listeriosis, Marburg virus disease, malaria, measles, meningitis, meningococcal disease, MERS, mumps, Nipah virus disease, pneumococcal disease, pneumonia, polio, rabies, Ross River fever, RSV infection, rubella, SARS, smallpox, shigellosis, shingles, sleeping sickness, streptococcal disease, tetanus, toxoplasmosis, tuberculosis, typhoid fever, West Nile fever, whooping cough, yellow fever, and yersiniosis.

87. The method or composition for use of claim 86, wherein the infectious disease is shingles.

88. The method of claim 79, or composition for use of claim 80, wherein the disease or condition is an autoimmune disease or condition selected from the group consisting of arthritis, diabetes, inflammatory bowel disease, multiple sclerosis, myasthenia gravis, pemphigus, psoriasis, system lupus erythematosus, and transplant rejection.

89. The method of claim 79, or composition for use of claim 80, wherein the disease or condition is cancer.

90. The method or use of claim 89, wherein the cancer is biliary tract cancer, bladder cancer, brain cancer, breast cancer, cervical cancer, choriocarcinoma, colorectal cancer, endometrial cancer, esophageal cancer, gastric cancer, leukemia, liver cancer, lymphoma, lung cancer, neuroblastoma, oral cancer, ovarian cancer, pancreatic cancer, prostate cancer, renal cancer, sarcoma, skin cancer, testicular cancer, or thyroid cancer.

91. The method of any one of claims 79 or 81 to 90, or composition for use of any one of claims 80 to 90, wherein the hydrogel composition is administered by injection to an individual in need thereof, and the injection is subcutaneous or intramuscular.

92. The method of any one of claims 79 or 81 to 91, or composition for use of any one of claims 80 to 91, wherein after administration of the hydrogel composition to the individual in need thereof, the blood serum of the individual has a therapeutic agent concentration of 50 ng/mL to 500 ng/mL, 100 ng/mL to 300 ng/mL, or 150 ng/mL to 250 ng/mL over a period of 1 day to 30 days, 1 day to 14 days, 1 day to 7 days, or 1 day to 3 days.

93. The method of any one of claims 79 or 81 to 91, or composition for use of any one of claims 80 to 91, wherein after administration of the hydrogel composition to the individual in need thereof, the blood serum of the individual has a therapeutic agent concentration of 50 ng/mL to 500 ng/mL, 100 ng/mL to 300 ng/mL, or 150 ng/mL to 250 ng/mL for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 21 days, at least 28 days, at least 1 month, at least 2 months, at least 3 months, at least 4 months, at least 5 months, at least 6 months, at least 9 months, or at least 12 months.

94. The method of any one of claims 79 or 81 to 91, or composition for use of any one of claims 80 to 91, wherein after administration of the hydrogel composition to the individual in need thereof, the blood serum of the individual has a therapeutic agent concentration of 50 ng/ml to 500 ng/mL, for at least 7 days or at least 14 days.

95. The method of claim 79, or composition for use of claim 80, wherein the method and use is for treating or preventing tissue adhesion in an individual in need thereof.

96. The method or composition for use of claim 95, wherein the hydrogel composition does not comprise a therapeutic agent.

97. The method or composition for use of claim 95, wherein the hydrogel composition comprises a therapeutic agent.

98. The method or composition for use of any one of claims 95 to 97, wherein the method or use comprises forming an incision in tissue of the individual in need thereof; and applying a hydrogel to tissue through the incision.

99. The method or composition for use of claim 98, wherein the forming the incision in the tissue is part of a surgical procedure.

100. The method or composition for use of any one of claims 95 to 99, wherein the hydrogel composition remains on the tissue for at least 7 days or at least 14 days after applying to the individual in need thereof.

101. The method or composition for use of any one of claims 95 to 100, wherein the hydrogel composition dissipates from the tissue in less than 120 days or less than 30 days after applying to the individual in need thereof.

102. The method or composition for use of any one of claims 95 to 101, wherein applying comprises spraying, spreading, or injecting the hydrogel composition onto the tissue.

103. The method or composition for use of any one of claims 95 to 102, wherein the tissue comprises abdominal, orthopedic, thoracic, gynecologic, or cardiac tissue.

104. A method for delivering a therapeutic agent to an individual in need thereof, comprising injecting the hydrogel composition according to any one of claims 1 to 78 into the individual in need thereof.

105. A hydrogel composition according to any one of claims 1 to 78 for use in 3D printing.

106. A method for forming an object, comprising depositing a hydrogel composition according to any one of claims 1 to 78 on a surface using a 3D printing process.

107. The method of claim 106, wherein the 3D printing process comprises an extrusion-based process.

108. The method of claim 107, wherein the extrusion-based process comprises extruding the hydrogel composition from a nozzle to form a filament.

109. The method of claim 108, wherein the filament has a diameter that is the same as or smaller than a diameter of the nozzle.

110. The method of claim 109, wherein the diameter of the filament is no more than 2 mm, 1 mm, 900 μm, 800 μm, 700 μm, 600 μm, 500 μm, 400 μm, 300 μm, 200 μm, 100 μm, 50 μm, 20 μm, or 10 μm.

111. The method of claim 109 or 110, wherein the diameter of the nozzle is at least 100 μm, 200 μm, 300 μm, 400 μm, 500 μm, 600 μm, 700 μm, 800 μm, 900 μm, 1 mm, or 2 mm.

112. The method of any one of claims 108 to 111, further comprising stretching the filament after extruding the filament from the nozzle to reduce the diameter of the filament.

113. The method of any one of claims 106 to 112, wherein the hydrogel composition comprises one or more reactive species, and the method further comprises applying energy to the hydrogel composition to cause the one or more reactive species to form a crosslinked polymer network.

114. The method of any one of claims 106 to 113, wherein the object is a tissue engineering scaffold, a drug delivery depot, or at least a portion of a medical device.
